(19) United States
(12) Patent Application Publication
(10) Pub. No.: US 2023/0374161 A1
(43) Pub. Date: Nov. 23, 2023

(54) COMPOSITIONS AND METHODS FOR INHIBITION OF NATURAL KILLER CELL RECEPTORS

(71) Applicant: The Board of Trustees of the Leland Stanford Junior University, Stanford, CA (US)

(72) Inventors: Kenan Christopher GARCIA, Menlo Park, CA (US); Junming REN, Palo Alto, CA (US)

(21) Appl. No.: 18/248,028

(22) PCT Filed: Oct. 15, 2021
(86) PCT No.: PCT/US2021/055185

§ 371 Date: Apr. 5, 2023

Related U.S. Application Data
(60) Provisional application No. 63/092,273, filed on Oct. 15, 2020.

(51) Int. Cl. C07K 16/46; C07K 16/28; A61P 37/02

(52) U.S. Cl. CPC C07K 16/468; C07K 16/286; A61P 37/02; C07K 2317/35; C07K 2317/52; C07K 2317/622

(57) ABSTRACT

The present disclosure relates generally to compositions and methods for modulating cell surface receptor signaling by specifically recruiting membrane phosphatases, in cis, to a spatial proximity of a natural killer cell receptor (NKR) molecule. More particularly, the disclosure provides novel multivalent protein-binding molecules that specifically bind NKR and antagonize the NKR-mediated signaling through recruitment of a phosphatase activity to dephosphorylate the intracellular domain of the NKR. Also provided are compositions and methods useful for producing such molecules, as well as methods for the treatment of health conditions associated with the inhibition of signal transduction mediated by NKRs.

### CROSS-REFERENCE TO RELATED APPLICATION

The present application claims priority to U.S. Provisional patent application Ser. No. 63/092,273, filed on Oct. 15, 2020, the disclosure of which is incorporated by reference herein in its entirety, including any drawings.

### STATEMENT REGARDING FEDERALLY SPONSORED R&D

This invention was made with Government support under contract CA177684 awarded by The National Institutes of Health. The Government has certain rights in the invention.

### INCORPORATION OF THE SEQUENCE LISTING

This application contains a Sequence Listing, which is hereby incorporated herein by reference in its entirety. The accompanying Sequence Listing text file, named “Sequence Listing_078430-526001WO_SequenceListing_ST25.txt,” was created on Oct. 5, 2021 and is 89 KB.

### FIELD

The present disclosure relates generally to the field of immuno-therapeutics, and particularly relates to multivalent protein-binding molecules designed to specifically bind a natural killer cell receptor (NKR) molecule and antagonize the NKR-mediated signaling through recruitment of a phosphatase activity. The disclosure also provides compositions and methods useful for the treatment of health conditions associated with the inhibition of signal transduction mediated by NKRs.

### BACKGROUND

Natural killer (NK) cells have held great promise for the immunotherapy of cancer for more than 3 decades. However, to date only modest clinical success has been achieved manipulating the NK cell compartment in patients with malignant disease. This is because NK cells express multiple receptors that are responsible for initiating activating or inhibitory signals. To be tolerant against healthy tissue and simultaneously attack infected cells, the activity of NK cells is tightly regulated by a sophisticated array of germline-encoded activating and inhibiting NK cell receptors (NKRs).

In recent years, significant progress has been made in the field of NKRs to help elucidate how NK cells selectively recognize and lyse tumor and virally infected cells while sparing normal cells. Major families of cell surface receptors that inhibit and activate NK cells to lyse target cells have been characterized. Further, identification of NK receptor ligands and their expression on normal and transformed cells completes the information has facilitate development of rational clinical approaches to manipulating receptor/ligand interactions for clinical benefit.

Targeting the NKR-mediated cell signaling represents a novel therapeutic approach to enhance anti-cancer immunity by promoting both innate and adaptive immune responses. For example, currently, for antagonism of NKR, the most prevalent strategy is through blockade of ligand binding between the extracellular domains (ECDs) of NKR through the use of, for example, antagonistic antibodies directed to an ECD of NKR. In this scenario, the blocking molecules (e.g., the antagonist antibodies) function by competing with the natural ligand for binding to the ECD of NKR. However, these blocking antibodies have been reported to be ineffective in many patients, and not capable of eliminating the basal intracellular signaling activity of NKR (also referred to as resting intracellular signaling activity) that signals through phosphorylation mechanisms. This failure to eliminate basal signaling activity frequently limits the effectiveness of ECD ligand blocking strategies. Thus, new methods are needed to directly reduce or eliminate the intracellular signaling of NKR by alternative mechanisms other than ECD ligand blocking mechanism which would reduce or eliminate both resting and ligand-activated signaling.

Accordingly, there remains a need for alternative approaches other than direct NKR-ligand blockade by antibodies or other agents, to complement existing therapeutic standards of care for immunotherapy of cancer and other immune diseases.

### SUMMARY

The present disclosure relates generally to the immuno-therapeutics, such as multivalent polypeptides, multivalent antibodies, and pharmaceutical compositions comprising the same for use in treating various health conditions, such as those associated with the inhibition of cell signaling mediated by a cell surface receptor of interest. In particular, as described in greater detail below, some embodiments of the disclosure provide compositions and methods for modulating cell signaling mediated by one or more NK cell receptors (NKRs) through, for example, specifically recruiting membrane phosphatases to a spatial proximity of the NKRs through, for example, direct ligation using a multivalent protein-binding agent. More particularly, the disclosure provides novel multivalent protein-binding molecules that specifically bind to one or more NKRs and thereby completely or partially antagonizing the NKR-mediated signaling through recruitment of a phosphatase activity. This approach, termed “Receptor Inhibition by Phosphatase Recruitment” (RIPR), was described previously in, for example, WO2019/222547A1. In some particular embodiments, the multivalent protein-binding molecules of the disclosure are multivalent polypeptides. In some embodiments, the multivalent polypeptides are multivalent antibodies. The disclosure also provides compositions and methods useful for producing such multivalent polypeptides, as well as methods for the treatment of health conditions associated with the inhibition of signal transduction mediated by NKRs.

In one aspect, provided herein are multivalent polypeptides which include: (a) a first amino acid sequence including a first polypeptide module capable of binding to a NK cell receptor (NKR) that signals through a phosphorylation mechanism; an (b) a second amino acid sequence including a second polypeptide module capable of binding to one or more receptor protein-tyrosine phosphatases (RPTPs) expressed on an immune cell that also expresses the NKR.

Non-limiting exemplary embodiments of the multivalent polypeptide of the disclosure can include one or more of the following features. In some embodiments, the immune cell is a natural killer (NK) cell or a T cell. In some embodiments, the immune cell is a NK cell. In some embodiments, the immune cell is a T cell. In some embodiments, the immune cell is a CD8+ T cell. In some embodiments, the one or more RPTPs includes CD45, CD148, or a functional variant of any thereof. In some embodiments, the NKR is an inhibiting NKR. In some embodiments, the inhibiting NKR is selected from the group consisting of killer immunoglobulin receptors KIR2DL, KIR3DL, NKG2A, NKG2B, NKG2E, NKG2F, NKp44, NKp30c, CD160, LAIR1, TIM-3, CD96, CEACAM1 (CEACAM5), KLRG-1, and TIGIT. In some embodiments, the NKR is an activating NKR. In some embodiments, the activating NKR is selected from the group consisting of NKp30a, NKp30b, NKp44, NKp46, NKG2D, NKG2C, KIR2DS, KIR3DS, KIR3DL4, DNAM-1, CD16, and CD161.

In some embodiments, at least one of the first and second polypeptide modules includes an amino acid sequence for a protein-binding ligand or an antigen-binding moiety. In some embodiments, the antigen-binding moiety is selected from the group consisting of a single-chain variable fragment (scFv), an antigen-binding fragment (Fab), a nanobody, a V_{H} domain, a V_{L} domain, a single domain antibody (dAb), a V_{NAR} domain, and a V_{H}H domain, a diabody, or a functional fragment of any thereof. In some embodiments, the protein-binding ligand includes an extracellular domain (ECD) of a NKR's natural ligand, an ECD of a cell surface receptor, or an ECD of a RPTP, or a functional variant of any thereof. In some embodiments, the protein-binding ligand includes one or more ECD of an MHC-I molecule (HLA) or a functional variant thereof.

In some embodiments, the first polypeptide module is operably linked to the second polypeptide module via a polypeptide linker sequence. In some embodiments, the multivalent polypeptide of the disclosure further includes an Fc region. As described in greater detail below, the Fc region included in the multivalent polypeptides of the disclosure for enhancing the half-life of the multivalent polypeptides in vivo (e.g., NKG2A-RIPR in mouse tumor models). In some embodiments, the Fc region is operably linked to the multivalent polypeptide via a polypeptide linker sequence. In some embodiments, the multivalent polypeptide of the disclosure further includes a third amino acid sequence comprising a third polypeptide module capable of binding to an antigen expressed on a CD8+ T cell.

In some embodiments, the multivalent polypeptide includes: (a) (i) an ECD of an MHC-I molecule, (ii) a polypeptide linker, and (iii) a CD45 scFv; (b) (i) a KIR2DL scFv, (ii) a polypeptide linker; and (iii) a CD45 scFv; (c) (i) a NKG2A scFv, (ii) a polypeptide linker; and (iii) a CD45 scFv; (d) (i) a KIR3DL scFv, (ii) a polypeptide linker; and (iii) a CD45 scFv; or (e) (i) a Ly49C/I scFv, (ii) a polypeptide linker; and (iii) a CD45 scFv. In some embodiments, such multivalent polypeptide further includes an Fc region. In some embodiments, the multivalent polypeptide of the disclosure includes an amino acid sequence that has at least 80% sequence identity to an amino acid sequence selected from the group consisting of SEQ ID NOS: 1-2, 32, 34, 36, 38, 40, 42, and 44.

In one aspect, provided herein are recombinant nucleic acid molecules including a nucleotide sequence encoding a multivalent polypeptide of the disclosure. In some embodiments, the nucleic acid molecules include a nucleotide sequence having at least 80% sequence identity to a nucleotide sequence selected from the group consisting of SEQ ID NOS: 5-6.

In another aspect, some embodiments disclosed herein relate to a recombinant cell including: (a) a multivalent polypeptide as disclosed herein, and/or (b) a recombinant nucleic acid molecule as disclosed herein. In some embodiments, the recombinant cell is an immune cell. In some embodiments, the immune cell expresses an NKR. In some embodiments, the immune cell is a NK cell or a T cell. In some embodiments, the immune cell is a NK cell. In some embodiments, the immune cell is a T cell. In some embodiments, the T cell is a CD8+ T cell.

In another aspect, some embodiments of the disclosure relate to pharmaceutical compositions which include a pharmaceutical acceptable excipient and one or more of the following: (a) a multivalent polypeptide of the disclosure; (b) a recombinant nucleic acid molecule of the disclosure; and (c) a recombinant cell of the disclosure.

In another aspect, disclosed herein are embodiments of methods for modulating cell signaling mediated by a NKR in a subject, the methods including administering to the subject a composition that includes one or more of the following: (a) a multivalent polypeptide of the disclosure; (b) a recombinant nucleic acid molecule of the disclosure; (c) a recombinant cell of the disclosure; and (d) a pharmaceutical composition of the disclosure.

In yet another aspect, provided herein are methods for treating a health condition in a subject in need thereof, the method including administering to the subject a composition that includes one or more of the following: (a) a multivalent polypeptide of the disclosure; (b) a recombinant nucleic acid molecule of the disclosure; (c) a recombinant cell of the disclosure; and (d) a pharmaceutical composition of the disclosure.

Non-limiting exemplary embodiments of the embodiments of the methods of the disclosure can include one or more of the following features. In some embodiments, the administered composition recruits the RPTP activity to a spatial proximity of a NKR, potentiates dephosphorylation of a NKR, and/or enhances NKR-mediated signaling. In some embodiments, the administered composition confers an enhancement in NK cell killing of a target cell (e.g., killing of the target cell by an NK cell expressing an NK receptor). In some embodiments, the subject has or is suspected of having a health condition associated with a natural killer cell receptor, e.g., inhibition of cell signaling mediated by a NKR. In some embodiments, the health condition is a cancer, an autoimmune disease, or a viral infection.

In some embodiments, the composition is administered to the subject individually (e.g., monotherapy) or as a first therapy in combination with a second therapy (e.g., multitherapy). In some embodiments, the second therapy is selected from the group consisting of chemotherapy, radiotherapy, immunotherapy, hormonal therapy, toxin therapy, or surgery. In some embodiments, the second therapy includes an anti-NKR antagonistic antibody.

In another aspect, some embodiments of the disclosure relate to kits for modulating cell signaling in a subject or for treating a health condition in a subject in need thereof, wherein the kits include one or more of the following: (a) a multivalent polypeptide of the disclosure; (b) a recombinant nucleic acid molecule of the disclosure; (c) a recombinant cell of the disclosure; and (d) a pharmaceutical composition of the disclosure.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 is a schematic depiction of ligand-independent (tonic) and ligand-induced signaling by the inhibitory NKR receptor. Antibody blockade of NKR interaction with HLA is expected to reduce ligand-induced but not ligand-independent signaling.

FIGS. 2A-2B schematically illustrate the mechanistic basis for inhibition of cell signaling mediated by an exemplary natural killer receptor, NKG2A, by using the RIPR methodology in accordance with some non-limiting embodiments of the disclosure. FIG. 2A graphically illustrates a non-limiting example of the modulation of cellular NKG2A-mediated signaling by in cis phosphatase recruitment in accordance with some embodiments of the disclosure. The recruitment of CD45 to NKG2A by NKR-RIPR at the cell surface of NK cells is anticipated to decrease phosphorylation of the receptor NKG2A. Recruitment of CD45 occurs in cis between proteins expressed at the cell membrane. CD45 recruitment is anticipated to decrease NKG2A phosphorylation. FIG. 2B schematically illustrates exemplary designs of αNKG2A-scFv and NKG2A-RIPR constructs in accordance with some non-limiting embodiments of the disclosure.

FIGS. 3A-3B schematically illustrate the mechanistic basis for inhibition of cell signaling mediated by another exemplary natural killer cell receptor, KIR2DL1, by using the RIPR methodology in accordance with some non-limiting embodiments of the disclosure. FIG. 3A graphically illustrates a non-limiting example of the modulation of cellular KIR2DL1-mediated signaling by in cis phosphatase recruitment in accordance with some embodiments of the disclosure. The recruitment of CD45 to KIR2DL1 by NKR-RIPR at the cell surface of NK cells is anticipated to decrease phosphorylation of the receptor KIR2DL1. FIG. 3B schematically illustrates exemplary designs of αKIR2DL-scFv and KIR2DL-RIPR constructs in accordance with some non-limiting embodiments of the disclosure.

FIGS. 4A-4C depict the design of an exemplary NKR-RIPR construct targeting NKG2A in accordance with some embodiments of the disclosure. FIG. 4A: Amino acid sequence of murine anti-NKG2A scFv and NKG2A-RIPR are shown. FIG. 4B: Binding affinity of murine anti-NKG2A scFv to human NKG2A. FIG. 4C: Murine anti-NKG2A scFv and NKG2A-RIPR were expressed in Hi5 cells and protein integrity and stability was analyzed by size-exclusion chromatography.

FIGS. 5A-5D depict the design of another exemplary NKR-RIPR construct targeting KIR2DL1 in accordance with some embodiments of the disclosure. FIG. 5A: Amino acid sequence of anti-KIR2DL scFv (human) and KIR2DL-RIPR are shown. FIG. 5B: Binding affinity of anti-KIR2DL scFv to human KIR2DL and KIR2DL3. FIGS. 5C-5D: Anti-KIR2DL scFv and KIR2DL-RIPR were expressed in Hi5 cells and protein integrity and stability was analyzed by size-exclusion chromatography.

FIGS. 6A-6B summarize the results of experiments performed to illustrate the surface expression of NKG2A on NK92 cells (FIG. 6A) and KIR2DL1 on NKL-KIR2DL1 cells. (FIG. 6B). In these experiments, surface expression was quantified by flow cytometry.

FIGS. 7A-7B summarize the results of experiments performed to illustrate that NKG2A-RIPR potentiates dephosphorylation of human NKG2A and enhances NK cell lysis of targets. FIG. 7A: HEK293 cells were transiently transfected with human HA-NKG2A, Lck, and human CD45, 24 hours after transfection, cells were left untreated (lane 1) or incubated for 20 min at 37° C. with anti-NKG2A scFv (lane 2 and 3) or NKG2A-RIPR (lane 4, 5) to induce recruitment of the CD45 phosphatase to the intracellular domains of NKG2A. A CD45_{dead} group was included for control purposes. After lysis, chimeric receptors were immunoprecipitated with anti-HA antibody directly conjugated to magnetic beads. Samples were probed for phosphotyrosine (pTyr) and HA by western blot. Data are representative of three independent biological repeats. FIG. 7B: Cell lysis by NKG2A-expressing cells, NK92 cells, against HLA-E positive K562 cells in the presence of 200 nM anti-NKG2A scFv or NKG2A-RIPR. Cell lysis was determined by flow cytometry.

FIGS. 8A-8B schematically summarize the results of experiments performed to illustrate that NKR-RIPR enhances lysis of target cell. FIG. 8A: NKR-RIPR construct targeting KIR2DL1 recruits the CD45 phosphatase to KIR2DL1. FIG. 8B: Cell lysis by NKL cells that express KIR2DL1 against HLA-Cw0304 positive 721.221 cells in the presence of 200 nM (top panel) or 1000 nM (bottom panel) anti-KIR2DL scFv or KIR2DL-RIPR. In these experiments, cell lysis was determined by flow cytometry.

FIGS. 9A-9B schematically summarize the results of experiments performed to illustrate the surface expression of CD8 on SKW3 cells (FIG. 9A) and the surface expression of KIR2DL1 on SKW3-KIR2DL1 cells (FIG. 9B). In these experiments, KIR2DL1 was lentivirally infected into CD8+SKW3 cells expressing the T cell receptor TCR55 and sorted for stable co-expression of TCR55 and KIR2DL1. Surface expression was quantified by flow cytometry.

FIGS. 10A-10B schematically summarize the results of experiments performed to illustrate that inhibition of NKR activity in NKR+CD8 T cells by anti-NKR antibody and NKR-RIPR enhances TCR signaling of the NKR+CD8 T cells. FIG. 10A: Expression of the NK receptor KIR2DL1 on CD8+ T cells could suppress T cell activation by peptide-WIC. Approximately 1×10⁴ 721.221 antigen-presenting cells expressing HLA-B35 and HLA-Cw0304 were pulsed with HIV peptide 20 for 3 hours at 37° C., then co-incubated 1:1 with SKW3 KIR2DL1(+) or SKW3 KIR2DL1(−) cell line for 16 hours. The response effect of HIV peptide was analyzed for CD69 expression by flow cytometry. FIG. 10B: blockade of KIR2DL1 with anti-KIR2DL scFv could partially reverse inhibition of T cell activation. NKR-RIPR construct targeting KIR2DL1 was found to nearly completely restore full CD8+ T cell activation (red trace) and with a superior efficacy compared to NKR inhibition by anti-KIR2DL scFv (green trace). In these experiments, HLA-B35/Cw0304 721.221 APC cells are pulsed with HIV peptide 20 for 3 hours, then co-incubated with SKW3 KIR2DL1(+) cell line in the presence 200 nM anti-KIR2DL scFv or KIR2DL-RIPR for 16 hours. CD69 activation of SKW3 T cells were tested.

FIGS. 11A-11G summarize results of experiments performed to illustrate that NKG2A-RIPR potentiates activation of NK and CD8+ cells. FIG. 11A schematically depicts the mechanistic basis for RIPR-mediated inhibition of NKG2A signaling in mouse. The binding of an NKG2A-RIPR construct to both CD45 and NKG2A results in recruitment of CD45 phosphatase to NKG2A, in cis, on the surface of a cell (e.g., NK cell or T cell). FIG. 11B summarizes results of experiments performed to demonstrate that 16A11-RIPR potentiates dephosphorylation of NKG2A in HEK293 cells. FIG. 11C summarizes results of experiments performed to optimize the linker length between 16A11 scFv and CD45 V_{H}H in the 16A11-RIPR construct. FIG. 11D shows the biased effect of 16A11-RIPR and 20D5 in NK cytotoxicity against Qa1(+) and Qa-1(−) cells. FIG. 11E shows that 20D5-RIPR is superior to 16A11-RIPR in NK killing of target cells. Fusion of mouse Fc keeps full activity of 16A11-RIPR in NK killing. FIGS. 11F and 11G show that NKG2A-RIPRs potentiate CD8+OT1 activation. 20D5 is a monoclonal antibody against mouse NKG2A.

FIGS. 12A-12B show that fusion of Fc potentiates the NK activity of both 5E6-scFv and 5E6-RIPR. FIG. 12A shows the design and expression of anti-Ly49C/I scFv (clone 5E6), 5E6-RIPR, 5E6-scFv-Fc, 5E6-RIPR-Fc. FIG. 12B shows that fusion of Fc potentiates the NK activity of both 5E6-scFv and 5E6-RIPR. 5E6 is a monoclonal antibody against mouse Ly49C/I (mouse version of KIR).

FIG. 13 shows that KIR3DL-RIPR potentiates the elimination of gliadin-specific CD4+ T cells by KIR+CD8+ T cells.

FIG. 14 shows schematic depictions and amino acid sequences of the following constructs, from top to bottom: anti-NKG2A scFv (clone 16A11), 16A11-RIPR, 16A11-scFv-Fc, and 16A11-RIPR-Fc. 16A11 is a monoclonal antibody against mouse NKG2A.

FIG. 15 shows schematic depictions and amino acid sequences of the following constructs, from top to bottom: anti-NKG2A scFv (clone 20D5), 20D5-RIPR, 20D5-scFv-Fc, and 20D5-RIPR-Fc.

FIG. 16 shows schematic depictions and amino acid sequences of the following constructs, from top to bottom: anti-Ly49C/I scFv (clone 5E6), 5E6-RIPR, 5E6-scFv-Fc, and 5E6-RIPR-Fc. Ly49C/I belongs to the killer-cell immunoglobulin-like (KIR) family in mouse, which is expressed in NK cells and CD8+ T cells and specifically bind MHC-I molecules.

FIG. 17 shows schematic depictions and amino acid sequences of the following constructs: anti-KIR3DL scFv (clone AZ158; top), and anti-KIR3DL-RIPR (bottom).

### DETAILED DESCRIPTION OF THE DISCLOSURE

The present disclosure generally relates to, inter alia, compositions and methods for modulating cell surface receptor signaling by specifically recruiting a membrane phosphatase activity to the spatial proximity of a NK cell receptor (NKR). This method for inhibiting NKR-mediated signaling represents an alternative approach to ECD ligand blockade. More particularly, the disclosure provides novel multivalent protein-binding molecules that specifically bind the NK cell receptor and antagonize the receptor's signaling, either completely or partially, through recruitment of a phosphatase activity, e.g., a transmembrane phosphatase. This approach, termed “Receptor Inhibition by Phosphatase Recruitment” (RIPR), was described previously in, for example, WO2019/222547A1. In particular, the experimental data presented herein has demonstrated that disruption of NKR-mediated signaling by RIPR constructs targeting a NKR (“NKR-RIPR”) results in signal inhibition and promotion of target cell lysis.

As described in greater detail below, a number of RIPR molecules capable of targeting NKRs, exemplified by KIR2DL1 or NKG2A, have been designed, constructed, and subsequently evaluated for their ability to enhance target cell lysis ex vivo.

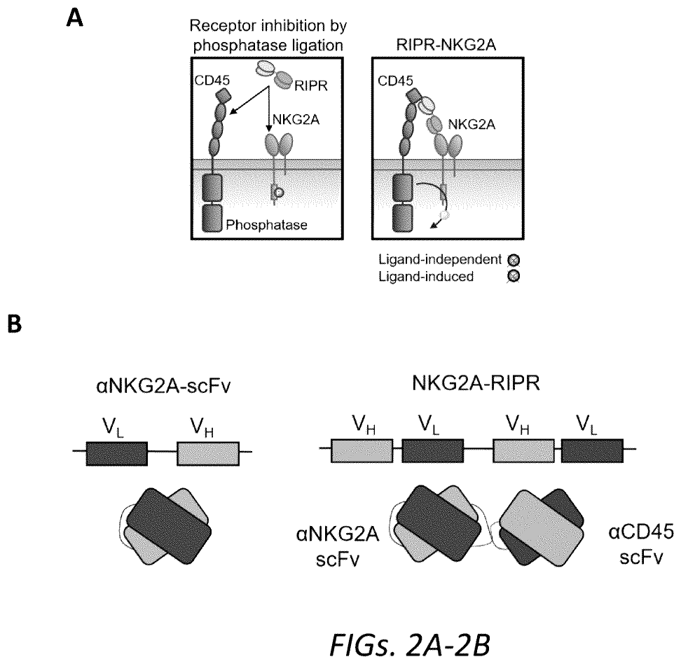

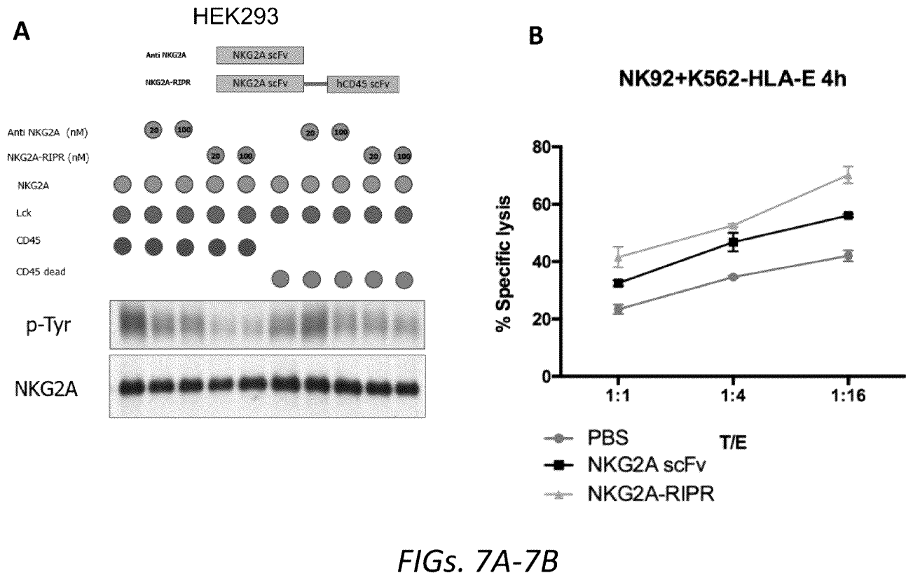

In some experiments described herein, an exemplary RIPR molecule capable of binding NKG2A was constructed, where an single chain antibody fragment (scFv) having binding affinity for the transmembrane receptor protein-tyrosine phosphatases CD45 was fused to a murine anti-NKG2A scFv having binding affinity for a human NKG2A to generate a NKR-RIPR construct targeting NKG2A (i.e., NKG2A-RIPR construct) (see, e.g., FIG. 2A-2B, 4A-4B, and Example 2). In an in vitro co-culture assay performed with human NK92 cell, the NKG2A-RIPR construct of the disclosure was observed to reduce NKG2A phosphorylation in a reconstitution assay in HEK293 cells that were transiently transfected with NKG2A, Lck, and CD45 (see, FIG. 7A). This exemplary NKG2A-RIPR construct was also found to be capable to enhance target cell lysis to higher levels than those achieved by a control sample containing an anti-NKG2A scFv alone (e.g., FIG. 7B).

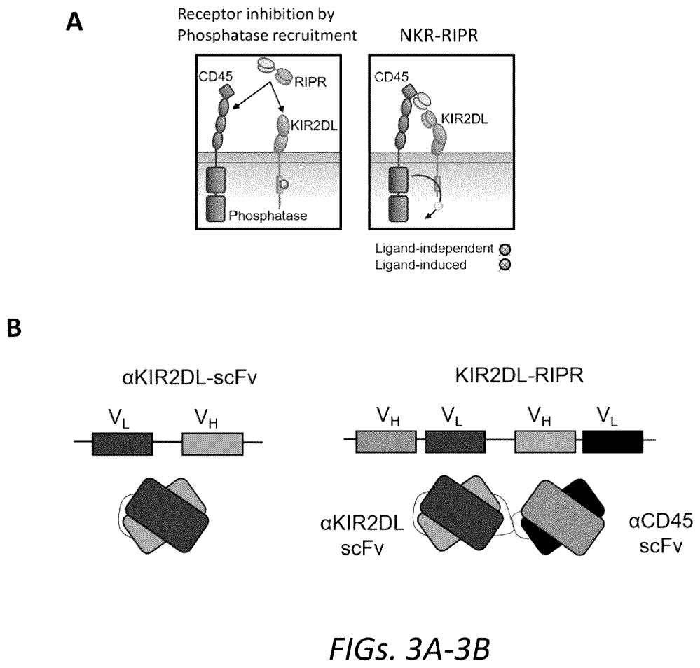

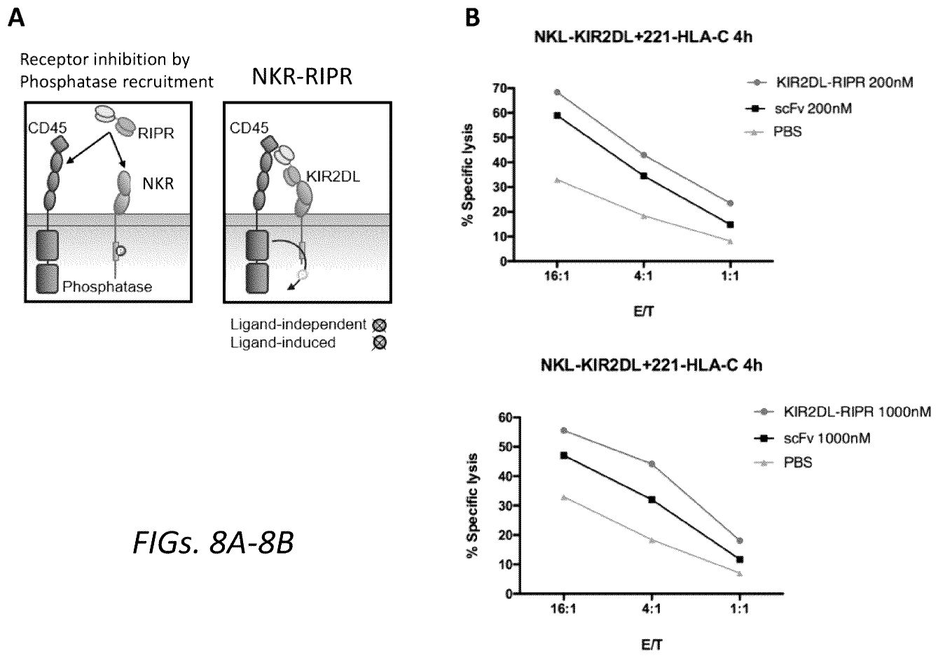

In some other experiments described below, another exemplary design of NKR-RIPR molecule was generated and composed of an anti-CD45 scFv fused to an anti-KIRD2L scFv having binding affinity for a human KIR2DL-1 and KIR2DL3, (see, KIRD2L-RIPR construct described in FIG. 3A-3B, 5A-B, and Example 2). This exemplary KIRD2L-RIPR construct was also found to be capable to enhance target cell lysis to higher levels than those achieved by a control sample containing an anti-KIRD2L scFv alone (e.g., FIG. 8).

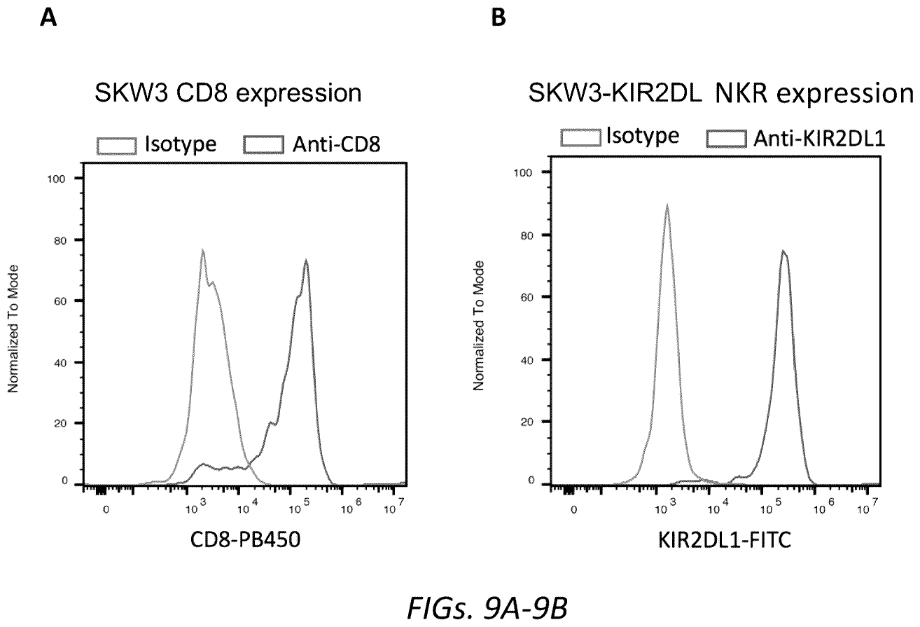

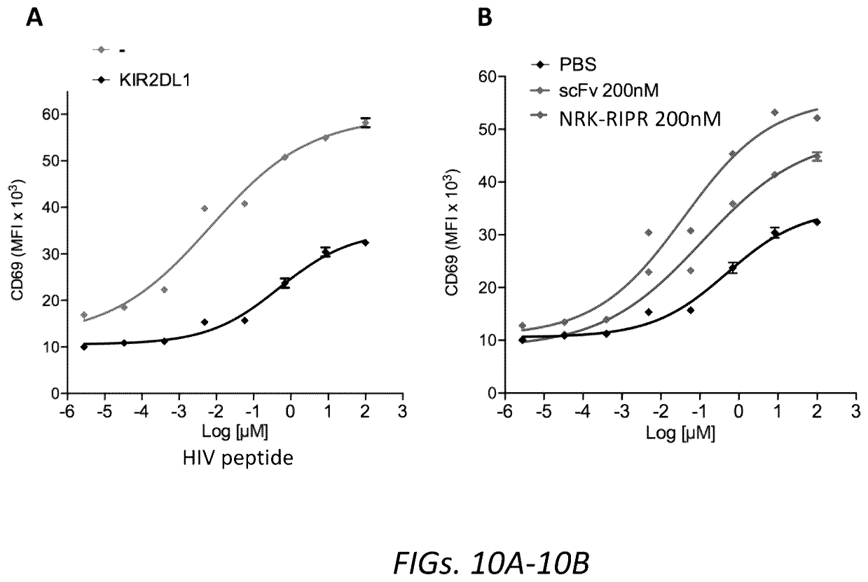

In some embodiments, the recruitment of phosphatase activity to the spatial proximity of a NKR is achieved via physical ligation. Some embodiments of the disclosure provide multivalent protein-binding molecules that are multivalent polypeptides (e.g., bivalent or trivalent) including a first polypeptide fragment capable of binding to a NK cell receptor (NKR) that signals through a phosphorylation mechanism, and a second polypeptide fragment capable of binding to one or more receptor protein-tyrosine phosphatases (RPTPs) expressed on an immune cell that also expresses the NKR. In some embodiments, the immune cell expressing the NKR is a NK cell. In some embodiments, the immune cell expressing the NKR is a T cell, such as, e.g., a regulatory T cell (Treg cell). In some embodiments, the Treg cell is a CD8+ Treg cell (see, e.g., Example 5, FIGS. 9A-9B and 10A-10B). The disclosure also relates to compositions and methods useful for producing such multivalent protein-binding molecules, as well as methods for the treatment of health conditions associated with the inhibition of signal transduction mediated by NKR.

As described in greater detail below, some embodiments of the present disclosure provide for, inter alia, engineered multivalent polypeptides, each exhibiting binding affinity to at least two cellular targets: a RPTP molecule and a NKR molecule. Without being bound by any particular theory, it is believed that the multivalent polypeptides disclosed herein are capable of recruiting the phosphatase activity encoded by RPTP molecule to the spatial proximity of the NKR molecule, subsequently reduces its phosphorylation. It is also believe that the multivalent polypeptides facilitate the modulation of the activity of the NKR molecule by binding to the extracellular domain of the NKR and the extracellular domain of a transmembrane phosphatase such that the intracellular domains of the NKR molecule and phosphatase are brought into sufficiently close proximity such that intracellular domain of the phosphatase dephosphorylates the intracellular domain of the NKR, thereby reducing the activity of the NKR molecule. In the case where the RPTP is CD45, ligation of a module which binds to the extracellular domain of the NKR molecule to a module which binds to the extracellular domain of the receptor protein-tyrosine phosphatase CD45 results in dephosphorylation of NKR, reduces NKR tonic signaling, and enhances target cell lysis. It is also believed that, without being bound by any particular theory, reducing the activity of NKR is expected to enhance target cell lysis and is useful as a therapy for a wide range of diseases, including cancer and chronic infection. This novel approach bypasses the current traditional strategy of regulating cellular receptor function through ligand blockade and allows regulating cellular receptor function by dephosphorylation of the receptor intracellular domain(s).

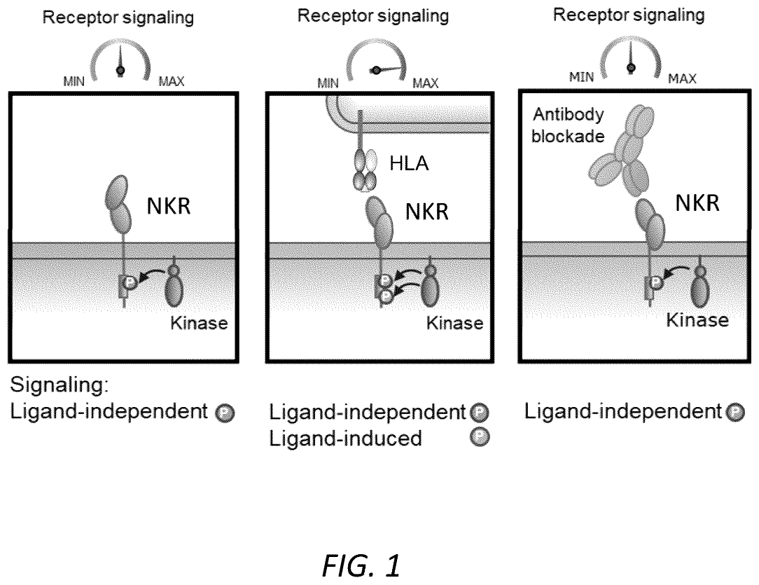

As discussed in greater detail below, it has been recognized that the current clinical options to modulate cell surface receptors is limited to ECD blocking antibodies, which block a receptor-ligand interaction from occurring at the surface of the cell. For example, in the case of NKRs, blocking the extracellular interaction between the NKRs and their natural ligands (e.g., MHC-I molecules or HLA) with high affinity antibodies has, to date, been the only available means to reduce NKR signaling. However, antibody blocking does not directly affect NKR phosphorylation and, importantly, does not reverse the basal, tonic, phosphorylation of NKR and sustained NKR from past interactions with HLA (see, e.g., FIG. 1). As discussed in greater detail herein, antibody blockade of NKR interaction with natural ligand is expected to reduce ligand-induced but not ligand-independent signaling. Anti-NKR or anti-ligand antibodies thus potentiate NK cell lysis of targets but not to the full extent due to the residual activity of the NKR intracellular domain (ICD). Without being bound by any particular theory, it is believed that existing blocking antibodies are not capable of completely eliminating NKR basal signaling in order to recover full immune cell activity. As described in some embodiments of the present disclosure, newly engineered multivalent antibodies address this problem by directly recruiting a phosphatase to dephosphorylate NKR. Accordingly, recruitment of RPTPs, and in particular of RPTPs expressed on the same immune cell that also expresses the target NKR represents a novel way to modulate the activity of NKRs of interest.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

The term “about”, as used herein, has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The terms “administration” and “administering”, as used herein, refer to the delivery of a bioactive composition or formulation by an administration route comprising, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

“Cancer” refers to the presence of cells possessing several characteristics typical of cancer-causing cells, such as uncontrolled proliferation, immortality, metastatic potential, rapid growth and proliferation rate, and certain characteristic morphological features. Cancer cells can aggregate into a mass, such as a tumor, or can exist alone within a subject. A tumor can be a solid tumor, a soft tissue tumor, or a metastatic lesion. As used herein, the term “cancer” also encompasses other types of non-tumor cancers. Non-limiting examples include blood cancers or hematological cancers, such as leukemia. Cancer can include premalignant, as well as malignant cancers.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

As used herein, the term “multivalent polypeptide” as used herein refers to a polypeptide comprising two or more protein-binding modules that are operably linked to each other. For example, a “bivalent” polypeptide of the disclosure includes two protein-binding modules, whereas a “trivalent” polypeptide of the disclosure includes three protein-binding modules. The amino acid sequences of the polypeptide modules may normally exist in separate proteins that are brought together in the multivalent polypeptide or they may normally exist in the same protein but are placed in a new arrangement in the multivalent polypeptide. A multivalent polypeptide may be created, for example, by chemical synthesis, or by creating and translating a polynucleotide in which the peptide regions are encoded in the desired relationship.

As used herein, and unless otherwise specified, a “therapeutically effective amount” or a “therapeutically effective number” of an agent is an amount or number sufficient to provide a therapeutic benefit in the treatment or management of a disease, e.g., cancer, or to delay or minimize one or more symptoms associated with the disease. A therapeutically effective amount or number of a compound means an amount or number of therapeutic agent, alone or in combination with other therapeutic agents, which provides a therapeutic benefit in the treatment or management of the disease. The term “therapeutically effective amount” can encompass an amount or number that improves overall therapy of the disease, reduces or avoids symptoms or causes of the disease, or enhances therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

As used herein, a “subject” or an “individual” includes animals, such as human (e.g., human subject) and non-human animals. In some embodiments, a “subject” or “individual” is a patient under the care of a physician. Thus, the subject can be a human patient or a subject who has, is at risk of having, or is suspected of having a disease of interest (e.g., cancer) and/or one or more symptoms of the disease. The subject can also be a subject who is diagnosed with a risk of the condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Activating and Inhibiting Natural Killer Cell Receptors (NRKs)

Natural killer (NK) cells are innate lymphocytes that play a crucial role in the early response to infection or malignant transformation. Under these conditions, NK cells are activated and promote target cell death through the release of cytolytic granules. In addition, they secrete cytokines that regulate the functions of other immune cells. The activity of NK cells is controlled by the relative balance of signals received from cell surface receptors that deliver either activating or inhibitory signals. Under normal physiological conditions, NK cell activation is inhibited by ligands expressed on healthy cells that engage inhibitory receptors on NK cells. A reduction in the expression of these ligands, which occurs in stressed cells, can lead to NK cell activation. NK cells can also be activated by the up-regulation of stress-induced ligands that typically occurs in response to infection or malignant transformation. Due to their ability to specifically attack and eliminate stressed cells, while maintaining tolerance to normal, healthy cells, NK cells are being investigated as potential anti-cancer agents.

NK cells express multiple receptors that are responsible for initiating activating or inhibitory signals. To be tolerant against healthy tissue and simultaneously attack infected cells, the activity of NK cells is tightly regulated by a sophisticated array of germline-encoded activating and inhibiting receptors.

Inhibiting NK cell receptors is characterized by the presence of immunoreceptor tyrosine-based inhibitory motifs (ITIM) in their cytoplasmic tail that can decrease the state of activation. Activating receptors lack ITIMs, but contain a positively charged amino acid (arginine or lysine) in their transmembrane region, and are associated with signaling adaptor molecules containing immunoreceptor tyrosine-based activating motifs (ITAM), such as DAP10, DAP12, or FcγR. NK cells integrate signals derived from both types of receptors upon cellular contact, thereby determining whether or not they should initiate effector functions. Many inhibiting NK cell receptors interact with major histocompatibility complex (MHC) class I proteins, which are ubiquitously expressed on the surface of nucleated cells. Because of the abundant expression of MHC-I on many cells, NK cells remain non-responsive to healthy tissue. But when cells have a decreased expression of MHC-I, which can occur during certain viral infections or in tumors, they can become target for NK cell killing. The process by which NK cells detect cells with aberrant MHC-I expression has been described as “missing-self” detection.

For the development of functional NK cells in the bone marrow, interactions between inhibiting receptors and MHC-I are necessary. This process is called NK cell education and determines the threshold for activation in mature NK cells. Depending on the strength of the inhibitory signals received during development, every NK cell balances its activation threshold as a rheostat to adapt to the particular MHC phenotype of their host. The expression of both activating and inhibiting receptors during development is thought to occur in a sequential and stochastic manner, giving rise to a large NK cell repertoire composed of 3000-35,000 functionally different NK cell subsets.

Activating NK cell receptors include members of the human killer immunoglobulin-like receptor (KIR) family or the mouse Ly49 family, CD94-NKG2C/E/H heterodimeric receptors, NKG2D, natural cytotoxicity receptors such as NKp30, NKp44, and NKp46, and the nectin/nectin-like binding receptors DNAM-1/CD226 and CRTAM.

In contrast, receptors that inhibit NK cell activation are important for self-tolerance. This group of receptors includes alternate members of the human KIR family or the mouse Ly49 family, CD94-NKG2A, and the nectin/nectin-like binding receptors TIGIT and CD96. In addition to these receptor families, there are multiple other receptors expressed by natural killer (NK) cells that regulate their activation. SLAM family receptors including 2B4/CD244, CRACC/SLAMF7, and NTB-A/SLAMF6, as well as Fc gamma RIIIA/CD16a, CD27, CD100/Semaphorin 4D, and CD160 are additional NK cell activating receptors, while the sialic acid-binding Siglecs (Siglec-3, -7, and -9), ILT2/LILRB1, KLRG1, LAIR-1, CD161/NKR-P1A, and CEACAM-1 are additional NK cell inhibitory receptors.

### Receptor Type Protein Tyrosine Phosphatases (RPTPs)

Reversible protein tyrosine phosphorylation is a major mechanism regulating cellular signaling that affects fundamental cellular events including metabolism, proliferation, adhesion, differentiation, migration, communication, and adhesion. For example, protein tyrosine phosphorylation determines protein functions, including protein-protein interactions, conformation, stability, enzymatic activity and cellular localization. Disruption of this key regulatory mechanism contributes to a variety of human diseases including cancer, diabetes, and auto-immune diseases. Net protein tyrosine phosphorylation is determined by the dynamic balance of the activity of protein tyrosine kinases (PTKs) and protein tyrosine phosphatases (PTPs). Aberrant regulation of the delicate balance between PTKs and PTPs is involved in the pathogenesis of a number of human diseases such as cancer, diabetes, and autoimmune diseases.

PTPs constitute a large and structurally diverse family of enzymes. Sequencing data indicate that there are 107 PTP genes in the human genome, of which 81 encode active protein phosphatases. Among the PTP super family, 38 are classical, tyrosine-specific PTPs, while the other 43 are dual-specificity tyrosine/serine, threonine phosphatases. The classical PTPs possess at least one catalytic domain known as the PTP domain. The 280-amino acid PTP catalytic domain contains an invariable active site signature motif (I/V)HCXAGXXR(S/T)G (SEQ ID NO: 10), which includes an essential cysteine that catalyzes nucleophilic attack on the phosphoryl group of its substrate and subsequent substrate dephosphorylation.

The PTPs can be further sub-divided into transmembrane receptor-like PTPs (RPTPs) and non-transmembrane PTPs based on their overall structure. Of these, receptor-type protein tyrosine phosphatases (RPTPs) are a family of integral cell surface proteins that possess intracellular PTP activity, and extracellular domains (ECDs) that have sequence homology to cell adhesion molecules (CAMs). Intracellular domains (ICDs) of most of the RPTPs contain two tandem PTP domains, termed D1 and D2. Generally, membrane proximal PTP domain (D1) possesses most of the catalytic activity, whereas membrane-distal PTP domain (D2) has weak, if any, catalytic activity. The ECDs of RPTPs contain combinations of CAM-like motifs with sequences homologous to fibronectin type III (FN3), meprin, A5, PTPμ (MAM), immunoglobulin (Ig), and carbonic anhydrase (CA). Collectively, the molecular structure of RPTPs enables direct coupling of extracellular adhesion-mediated events to regulation of intracellular signaling pathways.

Based on the structure of their ECDs, the RPTP family can be grouped into eight sub-families: R1/R6, R2A, R2B, R3, R4, R5, R7, and R8. Representative members of these sub-families include CD45, LAR, RPTP-κ, DEP-1, RPTP-α, RPTP-ζ, PTPRR, and IA2, respectively. Further information regarding the structural features that define each of the sub-families, their molecular/biochemical structure, mode of regulation, substrate specificity, and biological functions has been extensively documented and can be found in, e.g., Xu Y. et al. (J. Cell Commun. Signal. 6:125, 138, 2012) and Koncevic et al., InTechOpen, 2018.

CD45 and the R1/R6 Family

The receptor type protein tyrosine phosphatase CD45, also called the leukocyte common antigen (LCA), is the sole member of the R1/R6 subtype of RPTPs. CD45 is a type I transmembrane protein that is in various forms present on all differentiated hematopoietic cells, except erythrocytes and plasma cells, and assists in the activation of those cells (a form of co-stimulation). CD45 is expressed in lymphomas, B-cell chronic lymphocytic leukemia, hairy cell leukemia, and acute nonlymphocytic leukemia. Human CD45, which is encoded by the gene PTPRC, and is a cell membrane tyrosine phosphatase expressed by all cells of lymphoid origin, including hematopoietic cells, with the exception of platelets and erythrocytes, and functions as a key regulator of T and B cell signaling. Accordingly, CD45 is a suitable RPTP target for being recruited to many NKR-expressing cells, because CD45 is expressed on many types of immune cells that also express one or more NKRs. For examples, as discussed in greater detail below, the CD45RO isoform is expressed on activated and memory T cells, some B cell subsets, activated monocytes/macrophages, and granulocytes. The CD45RB isoform is expressed on peripheral B cells, naïve T cells, thymocytes, weakly on macrophages, and dendritic cells. In addition, NK cells have been also reported to express at least different isoforms of CD45 which are CD45RA and CD45RO.

CD45 consists of an extracellular region, short transmembrane segment and tandem PTP domains in the cytoplasmic region. Multiple isoforms of CD45 are generated by complex alternative splicing of exons in the extracellular domain of the molecule, which are expressed in a cell type specific manner depending on the cell differentiation and activation status. Non-limiting examples of CD45 isoforms include CD45RA, CD45RB, CD45RC, CD45RAB, CD45RAC, CD45RBC, CD45R0, CD45R (ABC). CD45RA is located on naive T cells and CD45R0 is located on memory T cells. CD45R is the longest protein and migrates at 200 kDa when isolated from T cells. B cells also express CD45R with heavier glycosylation, bringing the molecular weight to 220 kDa, hence the name B220; B cell isoform of 220 kDa. B220 expression is not restricted to B cells and can also be expressed on activated T cells, on a subset of dendritic cells and other antigen-presenting cells. Naive T lymphocytes express large CD45 isoforms and are usually positive for CD45RA. Activated and memory T lymphocytes express the shortest CD45 isoform, CD45R0, which lacks RA, RB, and RC exons. This shortest isoform is believed to facilitate T-cell activation.

CD45 plays important roles in immune system development and function and is required for antigen-specific lymphocyte stimulation and proliferation. CD45 regulates immune responses by controlling the TCR activation threshold, modulating cytokine responses, and regulating lymphocyte survival. All of these processes are essential in the pathogenesis of autoimmune and infectious diseases.

CD45 is a suitable RPTP target for being recruited to many immune receptors, because it will act on a broad range of substrates if they are brought into a spatial proximity of one to another, e.g. the two RPTP-binding and receptor-binding modules are in sufficient proximity to achieve dephosphorylation of the intracellular domain of the receptor. CD45 mediates T- and B-cell receptor function by regulating tyrosine phosphorylation of the Src family of PTKs (SFKs) like Lyn and Lck (lymphocyte-specific protein tyrosine kinase). CD45 dephosphorylates the inhibitory C-terminal phosphorylation site in Lyn and Lck, thereby potentiating the activity of these SFKs. Attenuation of SFK activity by CD45-mediated dephosphorylation of other tyrosines has also been reported. Studies in CD45 knockout mice show that CD45-mediated activation of Fyn and Lck is important in thymocyte development. Upon TCR ligation, activated Fyn and Lck phosphorylate components of the TCR complex like TCR-zeta and CD3-epsilon. These tyrosine-phosphorylated proteins provide docking sites for Src-homology 2 (SH2) domain-containing proteins to transmit down-stream signal. In CD45-null thymocytes, ligation of TCR does not lead to Lyn or Lck activation or to subsequent tyrosine phosphorylation of the TCR complex. Therefore, none of the down-stream signaling events occur; indicating the essential role of CD45 in TCR activation. CD45 has also been identified as a PTP that dephosphorylates the CD3-zeta and CD3-epsilon ITAMs, Janus kinases (JAKs) and negatively regulates cytokine receptor activation.

CD148 and the R3 Family

CD148 is a mammalian transmembrane protein, also referred to as DEP-1 (density enhanced phosphatase-1), ECRTP (endothelial cell receptor tyrosine phosphatase), PTP-eta, HPTPη, or BYP, depending upon species and cDNA origin. CD148 belongs to the R3 RPTP subfamily members which bear various numbers of FN3 domains in their ECDs and contain a single PTP domain in their ICD. Other members of the R3 subfamily include RPTP-β (encoded by the PTPRB gene), SAP1 (encoded by the PTPRH gene), GLEPP (encoded by the Ptpro gene) and PTPS31 (encoded by the PTPRP gene).

Similar to other RPTPs, CD148 has an intracellular carboxyl moiety with a catalytic domain, a single transmembrane domain, and an extracellular amino terminal domain (comprising at least five tandem fibronectin type II (FNIII) repeats, which have a folding pattern similar to that of Ig-like domains). The FNIII domains have an absolute specificity for phosphotyrosine residues, a high affinity for substrate proteins, and a specific activity which is several orders of magnitude greater than that of the PTKs. The FNIII domains are believed to participate in protein/protein interactions. Activation of CD148 triggers autophosphorylation of CD148, which transduces a biological signal resulting in inhibition of angiogenesis.

Disruption of the CD148 gene in transgenic mice leads to embryonic lethality with severe defects in vascular organization and enlarged vessels due to high endothelial cell proliferation. CD148 is associated with contact inhibition of VEGF-induced endothelial cell growth.

CD148 is a suitable RPTP target for being recruited to many NKR-expressing cells, because CD148 is expressed on many types of immune cells that also express one or more NKRs, such as T cells, B cells, NK cells, dendritic cells; granulocytes, macrophages, and monocytes.

### Compositions of the Disclosure

As described in greater detail below, one aspect of the present disclosure relates to multivalent protein-binding molecules that specifically bind one or more NKRs and thereby completely or partially antagonizing the NKR-mediated signaling through recruitment of a phosphatase activity, e.g., transmembrane phosphatase CD45. The disclosure also provides compositions and methods useful for producing such multivalent polypeptides, including (i) recombinant nucleic acids encoding such multivalent protein-binding molecules, (ii) recombinant cells that have been engineered to express a multivalent protein-binding molecule as disclosed herein.

### Multivalent Polypeptides and Multivalent Antibodies

In one aspect, some embodiments disclosed herein relate to a novel chimeric polypeptides containing multiple polypeptide modules, e.g., modular protein-binding moieties, each capable of binding to one or more target protein(s). In some embodiments, the disclosed chimeric polypeptide includes (a) a first amino acid sequence including a first polypeptide module capable of binding to a NKR that signals through a phosphorylation mechanism; an (b) a second amino acid sequence including a second polypeptide module capable of binding to one or more RPTPs expressed on an immune cell that also expresses the NKR. In some embodiments, the first polypeptide module is operably linked to the second polypeptide module. In some embodiment, the disclosed chimeric polypeptide is a multivalent polypeptide. In some embodiment, the multivalent polypeptide is a multivalent antibody. The binding of a first polypeptide module and a second polypeptide module to their respective target protein can be either in a competitive or non-competitive fashion with a natural ligand of the target protein. Accordingly, in some embodiments of the disclosure, the binding of a first polypeptide module and/or second polypeptide module to their respective target protein can be ligand-blocking. In some other embodiments, the binding of a first polypeptide module and/or second polypeptide module to their respective target protein does not block binding of the natural ligand.

Designation of the amino acid sequence of the multivalent polypeptide that includes a first polypeptide module capable of binding to a NKR molecule as the “first” amino acid sequence and the amino acid sequence of the multivalent polypeptide including a polypeptide module capable of binding to a RPTP as the “second” amino acid sequence is not intended to imply any particular structural arrangement of the “first” and “second” amino acid sequences within the multivalent polypeptide. By way of non-limiting example, in some embodiments of the disclosure, the multivalent polypeptide or multivalent antibody may include an N-terminal polypeptide module capable of binding to a NKR molecule and a C-terminal polypeptide module including a polypeptide capable of binding to a RPTP. In other embodiments, the multivalent polypeptide or multivalent antibody may include an N-terminal polypeptide module capable of binding to a RPTP and a C-terminal polypeptide module capable of binding to a NKR molecule. In addition or alternatively, the multivalent polypeptide or multivalent antibody may include more than one polypeptide module capable of binding to a NKR, and/or more than one polypeptide module capable of binding to a RPTP. Accordingly, in some embodiments, a first amino acid sequence of the multivalent polypeptide or multivalent antibody includes at least two, three, four, five, six, seven, eight, nine, or ten polypeptide modules each capable of binding to a NKR. In some embodiments, the at least two, three, four, five, six, seven, eight, nine, or ten polypeptide modules of a second amino acid sequence are each capable of binding to the same RPTP. In some embodiments, the at least two, three, four, five, six, seven, eight, nine, or ten polypeptide modules of a second amino acid sequence are each capable of binding to different RPTPs.

In addition or alternatively, as alluded to above, the multivalent polypeptides and antibodies as disclosed herein can incorporate both natural and unnatural amino acids at positions that affect the binding affinity of the multivalent polypeptides or multivalent antibodies with the respective target protein(s). As such, the binding affinity of the polypeptide modules to their respective target (e.g., RPTP or NKR) can be tuned to achieve a desired target cell specificity. For example, since CD45 is widely expressed, the NKR-binding module can be configured to form a high affinity binding module, while the CD45-binding module can be configured to have lower binding affinity. For instance, in some embodiments, a NKR-binding module has a higher affinity (lower K_{d}) to the NKR when compared to the binding affinity of the RPTP-binding module to the RPTP. In some embodiments, the difference in affinity is at least one order of magnitude or at least two orders of magnitude (e.g., the ratio of the K_{d} for the interaction of the RPTP-binding module to the RPTP to the K_{d} for the interaction of the NKR-binding module to the NKR is at least 10, at least 20, at least 50, or at least 100). One skilled in the art will appreciate that this concept of a multivalent polypeptide or multivalent antibody having high affinity for the RPTP or its target receptor (e.g., NKR), and lower affinity for the other can be an important part of tuning RIPR activity for target cell specificity. Accordingly, in some embodiments, the binding affinity of the RPTP-binding polypeptide module can be different from the binding affinity of the NKR-binding polypeptide module. For example, in some embodiments, the RPTP-binding polypeptide module has high affinity to its target (e.g., RPTP) and the NKR-binding polypeptide module has low affinity to its target (e.g., NKR). In some embodiments, the RPTP-binding polypeptide module has low affinity to its target and the NKR-binding polypeptide module has high affinity to its target. In some embodiments, the RPTP-binding and NKR-binding modules have the same affinity to the respective target proteins.

In some embodiments, the binding affinity of the NKR-binding and RPTP-binding modules each having an affinity for the extracellular domain of its respective target, is independently from K_{d}=10⁻⁵ to 10⁻¹² M, such as e.g., a K_{d} of about 10⁻⁵ to about 10⁻¹¹ M, alternatively a K_{d} of about 10⁻⁵ to about 10⁻¹⁰ M, alternatively a K_{d} of about 10⁻⁶ to about 10⁻¹² M, alternatively a K_{d} of about 10⁻⁷ to about 10⁻¹² M, alternatively a K_{d} of about 10⁻⁸ to about 10⁻¹² M, alternatively a K_{d} of about 10⁻⁹ to about 10⁻¹² M, alternatively a K_{d} of about 10⁻¹⁰ to about 10⁻¹² M, alternatively a K_{d} of about 10⁻¹¹ to about 10⁻¹² M, alternatively a K_{d} of about 10⁻⁵ to about 10⁻¹¹ M, alternatively a K_{d} of about 10⁻⁵ to about 10⁻¹⁰ M, alternatively a K_{d} of about 10⁻⁵ to about 10⁻⁹ M, alternatively a K_{d} of about 10⁻⁵ to about 10⁻⁸ M, alternatively a K_{d} of about 10⁻⁵ to about 10⁻⁷ M, alternatively a K_{d} of about 10⁻⁵ to about 10⁻⁶ M.

In some embodiments, the multivalent polypeptide or multivalent antibody as disclosed herein has a binding affinity for a RPTP (e.g., CD45) with a K_{d} of about 1,000 nM, about 800 nM, about 700 nM, about 600 nM, about 500 nM, about 400 nM, about 200 nM, about 100 nM, about 10 nM, about 5 nM, or about 1 nM. In some embodiments, the multivalent polypeptide or multivalent antibody as disclosed herein have low binding affinity for a RPTP, e.g. with a K_{d} of more than about 10⁻⁵ M, such as e.g., a K_{d} of more than about 10⁻⁴ M, more than about 10⁻³ M, more than about 10⁻² M, or more than about 10⁻¹ M. In some embodiments, the binding affinity (K_{d}) for a RPTP (e.g., CD45) can be about 700 nM. In some embodiments, the binding affinity of the multivalent polypeptide or multivalent antibody for CD45 can be about 300 nM.

In some embodiments, the multivalent polypeptide or multivalent antibody as disclosed herein can have binding affinity for a NKR molecule with a K_{d} of 1,000 nM, about 800 nM, about 700 nM, about 600 nM, about 500 nM, about 400 nM, about 200 nM, about 150 nM, about 100 nM, about 80 nM, about 60 nM, about 40 nM, about 20 nM, about 10 nM, about 5 nM, or about 1 nM. In some embodiments, the multivalent polypeptide or multivalent antibody as disclosed herein has a high binding affinity for a NKR molecule, e.g. with a K_{d} of less than about 10⁻⁸ M, less than about 10⁻⁹ M, less than about 10⁻¹⁰ M, less than about 10⁻¹¹ M, or less than about 10⁻¹² M. In some embodiments, the binding affinity of a multivalent polypeptide or multivalent antibody disclosed herein for a NKR molecule has a K_{d} of about 6.65×10⁻¹¹. In some embodiments, the binding affinity of a multivalent polypeptide or multivalent antibody disclosed herein for a NKR molecule has a K_{d} of about 4.3×10⁻¹⁰. In some embodiments, the binding affinity for a NKR molecule can be about 2.5×10⁻¹¹.

In some embodiments, a first amino acid sequence of the multivalent polypeptide or multivalent antibody is directly linked to a second amino acid sequence. In some embodiments, a first amino acid sequence is directly linked to a second amino acid sequence via at least one covalent bond. In some embodiments, a first amino acid sequence is directly linked to a second amino acid sequence via at least one peptide bond. In some embodiments, the C-terminal amino acid of a first amino acid sequence can be operably linked to the N-terminal amino acid of a second polypeptide module. Alternatively, the N-terminal amino acid of a first polypeptide module can be operably linked to the C-terminal amino acid of a second polypeptide module.

In some embodiments, a first amino acid sequence of the multivalent polypeptide or multivalent antibody is operably linked to a second amino acid sequence via a linker. There is no particular limitation on the linkers that can be used in the multivalent polypeptides described herein. In some embodiments, the linker is a synthetic compound linker such as, for example, a chemical cross-linking agent. Non-limiting examples of suitable cross-linking agents that are commercially available include N-hydroxysuccinimide (NHS), disuccinimidylsuberate (DSS), bis(sulfosuccinimidyl)suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES). Other examples of alterative structures and linkages suitable for the multivalent polypeptides and multivalent antibodies of the disclosure include those described in Spiess et al., Mol. Immunol. 67:95-106, 2015.

In some embodiments, a first amino acid sequence of a multivalent polypeptide or multivalent antibody disclosed herein is operably linked to a second amino acid sequence via a polypeptide linker (peptidal linkage). In some embodiments, the polypeptide linker comprising a single-chain polypeptide sequence comprising about one to 100 amino acid residues (e.g., 2, 3, 4, 5, 6, 7, 8, 9, 10, 11, 12, 13, 14, 15, 16, 17, 18, 19, 20, etc. amino acid residues) can be used as a polypeptide linker. In some embodiments, the linker polypeptide sequence includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the linker polypeptide sequence includes about 1 to 10, about 5 to 15, about 10 to 20, about 15 to 25, about 20 to 40, about 30 to 50, about 40 to 60, about 50 to 70 amino acid residues. In some embodiments, the linker polypeptide sequence includes about 40 to 70, about 50 to 80, about 60 to 80, about 70 to 90, or about 80 to 100 amino acid residues. In some embodiments, the linker polypeptide sequence includes about 1 to 10, about 5 to 15, about 10 to 20, about 15 to 25 amino acid residues.

In some embodiments, the length and amino acid composition of the linker polypeptide sequence can be optimized to vary the orientation and/or proximity of a first and a second polypeptide modules relative to one another to achieve a desired activity of the multivalent polypeptide. In some embodiments, the orientation and/or proximity of a first and a second polypeptide modules relative to one another can be varied as a “tuning” tool to achieve a tuning effect that would enhance or reduce the RPTP activity of the multivalent polypeptide. In some embodiments, the orientation and/or proximity of a first and a second polypeptide modules relative to one another can be optimized to create a partial antagonist to full antagonist versions of the bispecific polypeptide. In certain embodiments, the linker contains only glycine and/or serine residues (e.g., glycine-serine linker). Examples of such polypeptide linkers include: Gly, Ser; Gly Ser; Gly Gly Ser; Ser Gly Gly; Gly Gly Gly Ser (SEQ ID NO: 46); Ser Gly Gly Gly (SEQ ID NO: 47); Gly Gly Gly Gly Ser (SEQ ID NO: 23); Ser Gly Gly Gly Gly (SEQ ID NO: 24); Gly Gly Gly Gly Gly Ser (SEQ ID NO: 25); Ser Gly Gly Gly Gly Gly (SEQ ID NO: 26); Gly Gly Gly Gly Gly Gly Ser (SEQ ID NO: 27); Ser Gly Gly Gly Gly Gly Gly (SEQ ID NO: 28); (Gly Gly Gly Gly Ser)n (SEQ ID NO: 29), wherein n is an integer of one or more; and (Ser Gly Gly Gly Gly)n (SEQ ID NO: 30), wherein n is an integer of one or more. In some embodiments, the polypeptide linkers are modified such that the amino acid sequence Gly Ser Gly (GSG) (that occurs at the junction of traditional Gly/Ser linker polypeptide repeats) is not present. For example, in some embodiments, the polypeptide linker includes an amino acid sequence selected from the group consisting of: (GGGXX)nGGGGS (SEQ ID NO: 11) and GGGGS(XGGGS)n (SEQ ID NO: 12), where X is any amino acid that can be inserted into the sequence and not result in a polypeptide comprising the sequence GSG, and n is 0 to 4. In some embodiments, the sequence of a polypeptide linker is (GGGX1X2)nGGGGS (SEQ ID NO: 13) and X1 is P and X2 is S and n is 0 to 4. In some embodiments, the sequence of a polypeptide linker is (GGGX1X2)nGGGGS (SEQ ID NO: 14) and X1 is G and X2 is Q and n is 0 to 4. In some other embodiments, the sequence of a polypeptide linker is (GGGX1X2)nGGGGS (SEQ ID NO: 15) and X1 is G and X2 is A and n is 0 to 4. In some embodiments, the sequence of a polypeptide linker is GGGGS(XGGGS)n (SEQ ID NO: 16), and X is P and n is 0 to 4. In some embodiments, a linker polypeptide of the disclosure comprises or consists of the amino acid sequence (GGGGA)₂GGGGS (SEQ ID NO: 17). In some embodiments, a polypeptide linker comprises or consists of the amino acid sequence (GGGGQ)₂GGGGS (SEQ ID NO: 18). In some embodiments, a polypeptide linker comprises or consists of the amino acid sequence (GGGPS)₂GGGGS (SEQ ID NO: 19). In some embodiments, a polypeptide linker comprises or consists of the amino acid sequence GGGGS(PGGGS)₂ (SEQ ID NO: 20). In some embodiments, a polypeptide linker comprises or consists of an amino acid sequence set forth in SEQ ID NOs: 7-9 and 45 in the Sequence Listing.

In some embodiments, the multivalent polypeptide or multivalent antibody of the disclosure further includes an Fc region. In some embodiments, the Fc region is derived from mouse IgG1 with an N279Q mutation, which lacks glycosylation at N297 and does not bind mFcγR. In some embodiments, the Fc region is operably linked to the multivalent polypeptide or multivalent antibody via a polypeptide linker. In some embodiments, the polypeptide linker comprising a single-chain polypeptide sequence comprising about one to 100 amino acid residues (e.g., 2, 3, 4, 5, 6, 7, 8, 9, 10, 11, 12, 13, 14, 15, 16, 17, 18, 19, 20, etc. amino acid residues) can be used as a polypeptide linker. In some embodiments, the linker polypeptide sequence includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the linker polypeptide sequence includes about 1 to 10, about 5 to 15, about 10 to 20, about 15 to 25, about 20 to 40, about 30 to 50, about 40 to 60, about 50 to 70 amino acid residues. In some embodiments, the linker polypeptide sequence includes about 40 to 70, about 50 to 80, about 60 to 80, about 70 to 90, or about 80 to 100 amino acid residues. In some embodiments, the linker polypeptide sequence includes about 1 to 10, about 5 to 15, about 10 to 20, about 15 to 25 amino acid residues. In certain embodiments, the linker contains only glycine and/or serine residues (e.g., glycine-serine linker). Examples of such polypeptide linkers include: Gly, Ser; Gly Ser; Gly Gly Ser; Ser Gly Gly; Gly Gly Gly Ser (SEQ ID NO: 46); Ser Gly Gly Gly (SEQ ID NO: 47); Gly Gly Gly Gly Ser (SEQ ID NO: 23); Ser Gly Gly Gly Gly (SEQ ID NO: 24); Gly Gly Gly Gly Gly Ser (SEQ ID NO: 25); Ser Gly Gly Gly Gly Gly (SEQ ID NO: 26); Gly Gly Gly Gly Gly Gly Ser (SEQ ID NO: 27); Ser Gly Gly Gly Gly Gly Gly (SEQ ID NO: 28); (Gly Gly Gly Gly Ser)n (SEQ ID NO: 29), wherein n is an integer of one or more; and (Ser Gly Gly Gly Gly)n (SEQ ID NO: 30), wherein n is an integer of one or more. In some embodiments, the polypeptide linkers are modified such that the amino acid sequence Gly Ser Gly (GSG) (that occurs at the junction of traditional Gly/Ser linker polypeptide repeats) is not present. For example, in some embodiments, the polypeptide linker includes an amino acid sequence selected from the group consisting of: (GGGXX)nGGGGS (SEQ ID NO: 11) and GGGGS(XGGGS)n (SEQ ID NO: 12), where X is any amino acid that can be inserted into the sequence and not result in a polypeptide comprising the sequence GSG, and n is 0 to 4. In some embodiments, the sequence of a polypeptide linker is (GGGX1X2)nGGGGS (SEQ ID NO: 13) and X1 is P and X2 is S and n is 0 to 4. In some embodiments, the sequence of a polypeptide linker is (GGGX1X2)nGGGGS (SEQ ID NO: 14) and X1 is G and X2 is Q and n is 0 to 4. In some other embodiments, the sequence of a polypeptide linker is (GGGX1X2)nGGGGS (SEQ ID NO: 15) and X1 is G and X2 is A and n is 0 to 4. In some embodiments, the sequence of a polypeptide linker is GGGGS(XGGGS)n (SEQ ID NO: 16), and X is P and n is 0 to 4. In some embodiments, a linker polypeptide of the disclosure comprises or consists of the amino acid sequence (GGGGA)₂GGGGS (SEQ ID NO: 17). In some embodiments, a polypeptide linker comprises or consists of the amino acid sequence (GGGGQ)₂GGGGS (SEQ ID NO: 18). In some embodiments, a polypeptide linker comprises or consists of the amino acid sequence (GGGPS)₂GGGGS (SEQ ID NO: 19). In some embodiments, a polypeptide linker comprises or consists of the amino acid sequence GGGGS(PGGGS)₂ (SEQ ID NO: 20). In some embodiments, a polypeptide linker comprises or consists of an amino acid sequence set forth in SEQ ID NOs: 7-9 and 45 in the Sequence Listing.

In addition, or alternatively, in some embodiments, the multivalent polypeptides and multivalent antibodies of the disclosure can include one or more RPTP-binding modules chemically linked to one or more NKR-binding modules. In some embodiments, the multivalent polypeptides and multivalent antibodies of the disclosure can include (i) one or more RPTP-binding modules chemically linked to one or more NKR-binding modules; and (ii) one or more RPTP-binding modules linked to one or more NKR-binding modules via peptidyl linkages.

In some embodiments disclosed herein, at least one of the first and second polypeptide modules of the disclosed multivalent polypeptide or multivalent antibody includes an amino acid sequence for a protein-binding ligand or an antigen-binding moiety. In some embodiments, at least one of the first and second polypeptide modules includes an amino acid sequence for a protein-binding ligand. Generally, any suitable protein-binding ligands can be used for the compositions and methods of the present disclosure and can be, for example, any recombinant polypeptide or naturally-occurring polypeptide which has a specific binding affinity to a target antibody or a target protein (e.g., a recombinant or natural ligand of a RPTP or a NKR molecule). For example, non-limiting examples of suitable ligands for phosphatase CD45 include its natural ligands, such as e.g., lectin CD22 (Hermiston M L et al., Annu. Rev. Immunol. 2003) and Galactin-1 (Walzel H. et al., J. Immunol. Lett. 1999 and Nguyen J T et al. J Immunol. 2001). Non-limiting examples of suitable ligands for phosphatase CD148 include its natural ligands, such as e.g., Syndecan-2 and thrombospondin-1 (TSP1). In some embodiments, at least one of the first and second polypeptide modules of the disclosed multivalent polypeptide or multivalent antibody include an amino acid sequence for one or more extracellular domains (ECDs) of a NKR or of a RPTP. Accordingly, in some embodiments, a first polypeptide module of the disclosed multivalent polypeptide includes one or more ECDs of a NKR molecule operably linked to a second module of the multivalent polypeptide. Accordingly, in some embodiments, a first polypeptide module of the disclosed multivalent polypeptide includes one or more ECDs of a NKR ligand (e.g., MHC-I molecule) operably linked to a second module of the multivalent polypeptide. As discussed above, non-limiting examples of protein-binding ligands suitable for the compositions and methods of the disclosure include natural ligands of a NKR. For example, suitable natural ligands for NKR include HLA-A, HLA-A3/A11, HLA-Bw4, HLA-B, HLA-C1, HLA-C2, HLA-E, HLA-G, HSPG, heparin, vimentin, NKp44L, B7-H6, BAG-6, PCNA, MICA/B, and ULBP1-6. In some embodiments, a second polypeptide module of the disclosed multivalent polypeptide includes one or more ECDs of a RPTP operably linked to a first module of the multivalent polypeptide.

In addition or alternatively, the protein-binding ligand can be an agonist or an antagonist version of the target's natural ligand. Thus, in some embodiments, the protein-binding ligand is an agonist ligand of the RPTP or the NKR. In some other embodiments, the protein-binding ligand is an antagonist ligand of the RPTP or the NKR. In some embodiments, the protein-binding ligand can be a synthetic molecule such as, for example, peptides or small molecules.

In some embodiments, at least one of a first and a second polypeptide modules of the disclosed multivalent polypeptide or multivalent antibody includes an amino acid sequence for an antigen-binding moiety that binds to the target protein, e.g., a RPTP or a NKR. In some embodiments, the antigen-binding moiety includes one or more antigen-binding determinants of an antibody or a functional antigen-binding fragment thereof. Blocking antibodies and non-blocking antibodies are both suitable. As used herein, the term “blocking” antibody or an “antagonist” antibody refers to an antibody that prevents, inhibits, blocks, or reduces biological or functional activity of the antigen to which it binds. Blocking antibodies or antagonist antibodies can substantially or completely prevent, inhibit, block, or reduce the biological activity or function of the antigen. For example, a blocking anti-NKR antibody can prevent, inhibit, block, or reduce the binding interaction between NKR and its ligand, thus preventing, blocking, inhibiting, or reducing the immunosuppressive functions associated with the NKR/ligand interaction. The term “non-blocking” antibody refers to an antibody that does not interfere, inhibits, blocks, or reduces biological or functional activity of the antigen to which it binds.

The term “antigen-binding fragment” as used herein refers to an antibody fragment such as, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (e.g., bivalent diabody—bi-scFv or divalent diabody—di-scFv), or a multispecific antibody formed from a portion of an antibody including one or more complementarity-determining regions (CDRs) of the antibody. The antigen-binding moiety can include naturally-derived polypeptides, antibodies produced by immunization of a non-human animal, or antigen-binding moieties obtained from other sources, e.g., camelids (see, e.g., Bannas et al. Front. Immunol., 22 Nov. 2017; McMahon C. et al., Nat Struct Mol Biol. 25(3): 289-296, 2018). The antigen-binding moiety can be engineered, synthesized, designed, humanized (see, e.g., Vincke et al., J. Biol. Chem. 30; 284(5):3273-84, 2009), or modified so as to provide desired and/or improved properties.

Accordingly, in some embodiments, at least one of a first and a second polypeptide modules of the disclosed multivalent polypeptide or multivalent antibody includes an amino acid sequence for an antigen-binding moiety selected from the group consisting of antigen-binding fragments (Fab), single-chain variable fragments (scFv), nanobodies, V_{H} domains, V_{L} domains, single domain antibodies (dAb), V_{NAR} domains, and V_{H}H domains, diabodies, or a functional fragment of any one of the foregoing. One skilled in the art upon reading the present disclosure will readily understand that the term “functional fragment thereof” or “functional variant thereof” refers to a molecule having quantitative and/or qualitative biological activity in common with the wild-type molecule from which the fragment or variant was derived. For example, a functional fragment or a functional variant of an antibody is one which retains essentially the same ability to bind to the same epitope as the antibody from which the functional fragment or functional variant was derived. For instance, an antibody capable of binding to an epitope of a cell surface receptor may be truncated at the N-terminus and/or C-terminus, and the retention of its epitope binding activity assessed using assays known to those of skill in the art. In some embodiments, the antigen-binding moiety includes a single-chain variable fragment (scFv). In some embodiments, the antigen-binding moiety includes a diabody. In some embodiments, the antigen-binding moiety includes a bi-scFv or di-scFv, in which two scFv molecules are operably linked to each other. In some embodiments, the bi-scFv or di-scFv includes a single peptide chain with two V_{H} and two V_{L} regions, yielding tandem scFvs. In some embodiments, the antigen-binding moiety includes a nanobody. In some embodiments, the antigen-binding moiety includes a heavy chain variable region and a light chain variable region.

In some embodiments, the heavy chain variable region and the light chain variable region of the antigen-binding moiety are operably linked to each other via one or more intervening amino acid residues that are positioned between the heavy chain variable region and the light chain variable region. In some embodiments, the one or more intervening amino acid residues include a linker polypeptide sequence. In some embodiments, polypeptide linker includes about one to 100 amino acid residues (e.g., 2, 3, 4, 5, 6, 7, 8, 9, 10, 11, 12, 13, 14, 15, 16, 17, 18, 19, 20, etc. amino acid residues) can be used as a polypeptide linker. In some embodiments, the linker polypeptide sequence includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the linker polypeptide sequence includes about 1 to 10, about 5 to 15, about 10 to 20, about 15 to 25, about 20 to 40, about 30 to 50, about 40 to 60, about 50 to 70 amino acid residues. In some embodiments, the linker polypeptide sequence includes about 40 to 70, about 50 to 80, about 60 to 80, about 70 to 90, or about 80 to 100 amino acid residues. In some embodiments, the linker polypeptide sequence includes about 1 to 10, about 5 to 15, about 10 to 20, about 15 to 25 amino acid residues. In some embodiments, the length and amino acid composition of the linker polypeptide sequence can be optimized to vary the orientation and/or proximity of a first and a second polypeptide modules relative to one another to achieve a desired activity of the multivalent polypeptide. In some embodiments, the orientation and/or proximity of a first and a second polypeptide modules relative to one another can be varied as a “tuning” tool or effect that would enhance or reduce the RPTP activity of the multivalent polypeptide. In some embodiments, the orientation and/or proximity of a first and a second polypeptide modules relative to one another can be optimize to create a partial antagonist to full antagonist versions of the multivalent polypeptide.

In certain embodiments, the linker contains only glycine and/or serine residues (e.g., glycine-serine linker). Examples of such polypeptide linkers include: Gly, Ser; Gly Ser; Gly Gly Ser; Ser Gly Gly; Gly Gly Gly Ser; Ser Gly Gly Gly; Gly Gly Gly Gly Ser; Ser Gly Gly Gly Gly; Gly Gly Gly Gly Gly Ser; Ser Gly Gly Gly Gly Gly; Gly Gly Gly Gly Gly Gly Ser; Ser Gly Gly Gly Gly Gly Gly; (Gly Gly Gly Gly Ser)n, wherein n is an integer of one or more; and (Ser Gly Gly Gly Gly)n, wherein n is an integer of one or more. In some embodiments, the linker polypeptides are modified such that the amino acid sequence GSG (that occurs at the junction of traditional Gly/Ser linker polypeptide repeats) is not present. For example, in some embodiments, the polypeptide linker includes an amino acid sequence selected from the group consisting of: (GGGXX)nGGGGS and GGGGS(XGGGS)n, where X is any amino acid that can be inserted into the sequence and not result in a polypeptide including the sequence GSG, and n is 0 to 4. In some embodiments, the sequence of a linker polypeptide is (GGGX1X2)nGGGGS and X1 is P and X2 is S and n is 0 to 4. In some other embodiments, the sequence of a linker polypeptide is (GGGX1X2)nGGGGS and X1 is G and X2 is Q and n is 0 to 4. In some other embodiments, the sequence of a linker polypeptide is (GGGX1X2)nGGGGS and X1 is G and X2 is A and n is 0 to 4. In yet some other embodiments, the sequence of a linker polypeptide is GGGGS(XGGGS)n, and X is P and n is 0 to 4. In some embodiments, a linker polypeptide of the disclosure comprises or consists of the amino acid sequence (GGGGA)2GGGGS. In some embodiments, a linker polypeptide comprises or consists of the amino acid sequence (GGGGQ)2GGGGS. In some embodiments, a linker polypeptide comprises or consists of the amino acid sequence (GGGPS)2GGGGS. In some embodiments, a linker polypeptide comprises or consists of the amino acid sequence GGGGS(PGGGS)2. In yet some embodiments, a linker polypeptide comprises or consists of an amino acid sequence set forth in SEQ ID NOs: 7-9 and 45 in the Sequence Listing.

In some embodiments, a first polypeptide module of the multivalent polypeptides and multivalent antibodies disclosed herein includes an antigen-binding moiety capable of binding one or more target NKRs. Both activating NKRs and inhibiting NKRs are suitable targets. Non-limiting examples of suitable NKRs include inhibiting KIRs such as killer immunoglobulin receptors KIR2DL and KIR3DL, as well as NKG2A, NKG2B, NKG2E, NKG2F, NKp44, NKp30c, CD160, LAIR1, TIM-3, CD96, CEACAM1 (CEACAM5), KLRG-1, and TIGIT. In some embodiments, at least one of the inhibiting KIRs is KIR2DL. In some embodiments, the KIR2DL receptor is KIR2DL1, KIR2DL2, or KIR2DL3. In some embodiments, the at least one of the inhibiting NKRs is KIR3DL. In some embodiments, the KIR3DL receptor is KIR3DL1 or KIR3DL2. In some embodiments, the at least one of the inhibiting NKRs is NKG2A. In some embodiments, the KIR2DL receptor is KIR2DL1, KIR2DL2, or KIR2DL3. In some embodiments, the KIR3DL receptor is KIR3DL1 or KIR3DL2. As such, in some embodiments of the disclosure, the multivalent polypeptides and multivalent antibodies disclosed herein includes an antigen-binding moiety capable of binding one or more inhibiting NKRs. In some embodiments, the one or more inhibiting NKRs is selected from the group consisting of killer immunoglobulin receptors KIR2DL, KIR3DL, NKG2A, NKG2B, NKG2E, NKG2F, NKp44, NKp30c, CD160, LAIR1, TIM-3, CD96, CEACAM1 (CEACAM5), KLRG-1, and TIGIT. In some embodiments, the one or more inhibiting NKRs includes NKG2A, KIR2D1, and/or KIR2DL3.

Suitable activating NKRs include, but are not limited to, NKp30a, NKp30b, NKp44, NKp46, NKG2D, NKG2C, KIR2DS, KIR3DS, KIR3DL4, DNAM-1, CD16, and CD161. In some embodiments, the at least one of the activating NKRs is KIR2DS. In some embodiments, the KIR2DS receptor is KIR2DS1, KIR2DS2, or KIR2DS4. As such, in some embodiments of the disclosure, the multivalent polypeptides and multivalent antibodies disclosed herein includes an antigen-binding moiety capable of binding one or more activating NKRs. In some embodiments, the one or more activating NKRs is selected from the group consisting of NKp30a, NKp30b, NKp44, NKp46, NKG2D, NKG2C, KIR2DS, KIR3DS, KIR3DL4, DNAM-1, CD16, and CD161.

In some embodiments, a second polypeptide module of the multivalent polypeptides and multivalent antibodies disclosed herein includes an antigen-binding moiety capable of binding one or more target RPTPs. Non-limiting examples of suitable RPTPs include members of sub-families R1/R6. In some embodiments, a second polypeptide module of the multivalent polypeptides and multivalent antibodies disclosed herein includes an antigen-binding moiety capable of binding CD45 phosphatase or a functional variant thereof, such as e.g., a homolog thereof. In some embodiments, the CD45 phosphatase is a human CD45 phosphatase. In general, any isoforms of CD45 can be used. In some embodiments, the RPTP is a CD45 isoform selected from the group consisting of CD45RA, CD45RB, CD45RC, CD45RAB, CD45RAC, CD45RBC, CD45R0, and CD45R. Exemplary CD45-binding moieties suitable for the compositions and methods disclose herein include, but are not limited to those described in U.S. Pat. Nos. 7,825,222 and 9,701,756. Additional suitable RPTPs include members of sub-families R3. In some embodiments, a second polypeptide module of the multivalent polypeptides and multivalent antibodies disclosed herein includes an antigen-binding moiety capable of binding CD148 phosphatase or a functional variant thereof, such as e.g., a homolog thereof. In some embodiments, the CD148 phosphatase is a mammalian CD148 phosphatase. In some embodiments, the CD148 phosphatase is a human CD148 phosphatase.

Non-limiting exemplary embodiments of the multivalent polypeptide of the disclosure can include one or more of the following features. In some embodiments, the one or more RPTPs includes CD45, CD148, or a functional variant of any thereof. In some embodiments, at least one of the first and second polypeptide modules includes an amino acid sequence for a protein-binding ligand or an antigen-binding moiety. In some embodiments, the antigen-binding moiety is selected from the group consisting of a single-chain variable fragment (scFv), an antigen-binding fragment (Fab), a nanobody, a V_{H} domain, a V_{L} domain, a single domain antibody (dAb), a V_{NAR} domain, and a V_{H}H domain, a diabody, or a functional fragment of any thereof. In some embodiments, the protein-binding ligand includes an extracellular domain (ECD) of a NKR's natural ligand, an ECD of a cell surface receptor, or an ECD of a RPTP, or a functional variant of any thereof. In some embodiments, the protein-binding ligand includes one or more ECD of an MHC-I molecule (HLA) or a functional variant thereof. In some embodiments, the first polypeptide module is operably linked to the second polypeptide module via a polypeptide linker sequence.

In some embodiments, the multivalent polypeptide of the disclosure further includes a third amino acid sequence comprising a third polypeptide module capable of binding to an antigen expressed on a CD8+ T cell. In some embodiments, the third polypeptide module includes an antigen-binding moiety having binding affinity for CD8. In some embodiments, the third polypeptide module includes an antigen-binding moiety having binding affinity for CD8⁺NKR⁺ T cells.

In some embodiments, a multivalent polypeptide of the disclosure includes: (i) an ECD of an MHC-I molecule, (ii) a polypeptide linker, and (iii) a CD45 scFv. In some embodiments, a multivalent polypeptide of the disclosure includes: (i) a KIR2DL scFv, (ii) a polypeptide linker; and (iii) a CD45 scFv. In some embodiments, a multivalent polypeptide of the disclosure includes: (i) a NKG2A scFv, (ii) a polypeptide linker; and (iii) a CD45 scFv. In some embodiments, a multivalent polypeptide of the disclosure includes: (i) a KIR3DL scFv, (ii) a polypeptide linker; and (iii) a CD45 scFv. In some embodiments, a multivalent polypeptide of the disclosure includes: (i) a Ly49C/I scFv, (ii) a polypeptide linker; and (iii) a CD45 scFv.

In some embodiments, the NKG2A scFv includes an amino acid sequence that has at least 80%, at least 85%, at least 90%, at least 95%, at least 96%, at least 97%, at least 98%, or at least 99% sequence identity to the amino acid sequence of SEQ ID NO: 3. In some embodiments, the NKG2A scFv includes the amino acid sequence of SEQ ID NO: 3, wherein one, two, three, four, or five of the amino acid residues in the sequence of SEQ ID NO: 3 is substituted by a different amino acid residue. In some embodiments, the KIR2DL scFv includes an amino acid sequence that has at least 80%, at least 85%, at least 90%, at least 95%, at least 96%, at least 97%, at least 98%, or at least 99% sequence identity to the amino acid sequence of SEQ ID NO: 4. In some embodiments, the KIR2DL scFv includes the amino acid sequence of SEQ ID NO: 4, wherein one, two, three, four, or five of the amino acid residues in the sequence of SEQ ID NO: 4 is substituted by a different amino acid residue.

In some embodiments, the Ly49C/I scFv includes an amino acid sequence that has at least 80%, at least 85%, at least 90%, at least 95%, at least 96%, at least 97%, at least 98%, or at least 99% sequence identity to the amino acid sequence of SEQ ID NO: 39. In some embodiments, the KIR3DL scFv includes the amino acid sequence of SEQ ID NO: 39, wherein one, two, three, four, or five of the amino acid residues in the sequence of SEQ ID NO: 39 is substituted by a different amino acid residue.

In some embodiments, the KIR3DL scFv includes an amino acid sequence that has at least 80%, at least 85%, at least 90%, at least 95%, at least 96%, at least 97%, at least 98%, or at least 99% sequence identity to the amino acid sequence of SEQ ID NO: 43. In some embodiments, the KIR3DL scFv includes the amino acid sequence of SEQ ID NO: 43, wherein one, two, three, four, or five of the amino acid residues in the sequence of SEQ ID NO: 43 is substituted by a different amino acid residue.

In some embodiments, a multivalent polypeptide of the disclosure includes an amino acid sequence that has at least 80%, at least 85%, at least 90%, at least 95%, at least 96%, at least 97%, at least 98%, or at least 99% sequence identity to an amino acid sequence selected from the group consisting of SEQ ID NOS: 1-2, 32, 34, 36, 38, 40, 42, 44. In some embodiments, a multivalent polypeptide of the disclosure includes an amino acid sequence that has at least 80%, at least 85%, at least 90%, at least 95%, at least 96%, at least 97%, at least 98%, or at least 99% sequence identity to the amino acid sequence of SEQ ID NO: 1. In some embodiments, the multivalent polypeptide of the disclosure includes the amino acid sequence of SEQ ID NO: 1, wherein one, two, three, four, or five of the amino acid residues in the sequence of SEQ ID NO: 1 is substituted by a different amino acid residue. In some embodiments, a multivalent polypeptide of the disclosure includes an amino acid sequence that has at least 80%, at least 85%, at least 90%, at least 95%, at least 96%, at least 97%, at least 98%, or at least 99% sequence identity to the amino acid sequence of SEQ ID NO: 2. In some embodiments, the multivalent polypeptide of the disclosure includes the amino acid sequence of SEQ ID NO: 2, wherein one, two, three, four, or five of the amino acid residues in the sequence of SEQ ID NO: 2 is substituted by a different amino acid residue. In some embodiments, a multivalent polypeptide of the disclosure includes an amino acid sequence that has at least 80%, at least 85%, at least 90%, at least 95%, at least 96%, at least 97%, at least 98%, or at least 99% sequence identity to the amino acid sequence of SEQ ID NO: 32. In some embodiments, the multivalent polypeptide of the disclosure includes the amino acid sequence of SEQ ID NO: 32, wherein one, two, three, four, or five of the amino acid residues in the sequence of SEQ ID NO: 32 is substituted by a different amino acid residue. In some embodiments, a multivalent polypeptide of the disclosure includes an amino acid sequence that has at least 80%, at least 85%, at least 90%, at least 95%, at least 96%, at least 97%, at least 98%, or at least 99% sequence identity to the amino acid sequence of SEQ ID NO: 34. In some embodiments, the multivalent polypeptide of the disclosure includes the amino acid sequence of SEQ ID NO: 34, wherein one, two, three, four, or five of the amino acid residues in the sequence of SEQ ID NO: 34 is substituted by a different amino acid residue.

In some embodiments, a multivalent polypeptide of the disclosure includes an amino acid sequence that has at least 80%, at least 85%, at least 90%, at least 95%, at least 96%, at least 97%, at least 98%, or at least 99% sequence identity to the amino acid sequence of SEQ ID NO: 36. In some embodiments, the multivalent polypeptide of the disclosure includes the amino acid sequence of SEQ ID NO: 36, wherein one, two, three, four, or five of the amino acid residues in the sequence of SEQ ID NO: 36 is substituted by a different amino acid residue. In some embodiments, a multivalent polypeptide of the disclosure includes an amino acid sequence that has at least 80%, at least 85%, at least 90%, at least 95%, at least 96%, at least 97%, at least 98%, or at least 99% sequence identity to the amino acid sequence of SEQ ID NO: 38. In some embodiments, the multivalent polypeptide of the disclosure includes the amino acid sequence of SEQ ID NO: 38, wherein one, two, three, four, or five of the amino acid residues in the sequence of SEQ ID NO: 38 is substituted by a different amino acid residue.

In some embodiments, a multivalent polypeptide of the disclosure includes an amino acid sequence that has at least 80%, at least 85%, at least 90%, at least 95%, at least 96%, at least 97%, at least 98%, or at least 99% sequence identity to the amino acid sequence of SEQ ID NO: 40. In some embodiments, the multivalent polypeptide of the disclosure includes the amino acid sequence of SEQ ID NO: 40, wherein one, two, three, four, or five of the amino acid residues in the sequence of SEQ ID NO: 40 is substituted by a different amino acid residue. In some embodiments, a multivalent polypeptide of the disclosure includes an amino acid sequence that has at least 80%, at least 85%, at least 90%, at least 95%, at least 96%, at least 97%, at least 98%, or at least 99% sequence identity to the amino acid sequence of SEQ ID NO: 42. In some embodiments, the multivalent polypeptide of the disclosure includes the amino acid sequence of SEQ ID NO: 42, wherein one, two, three, four, or five of the amino acid residues in the sequence of SEQ ID NO: 42 is substituted by a different amino acid residue. In some embodiments, a multivalent polypeptide of the disclosure includes an amino acid sequence that has at least 80%, at least 85%, at least 90%, at least 95%, at least 96%, at least 97%, at least 98%, or at least 99% sequence identity to the amino acid sequence of SEQ ID NO: 44. In some embodiments, the multivalent polypeptide of the disclosure includes the amino acid sequence of SEQ ID NO: 44, wherein one, two, three, four, or five of the amino acid residues in the sequence of SEQ ID NO: 44 is substituted by a different amino acid residue.

In some embodiments, a multivalent polypeptide of the disclosure includes an amino acid sequence that has 100% sequence identity to an amino acid sequence selected from the group consisting of SEQ ID NOS: 1-2, 32, 34, 36, 38, 40, 42, and 44. In some embodiments, a multivalent polypeptide of the disclosure includes an amino acid sequence that has 100% sequence identity to the amino acid sequence of SEQ ID NO: 1. In some embodiments, a multivalent polypeptide of the disclosure includes an amino acid sequence that has 100% sequence identity to the amino acid sequence of SEQ ID NO: 2. In some embodiments, a multivalent polypeptide of the disclosure includes an amino acid sequence that has 100% sequence identity to the amino acid sequence of SEQ ID NO: 32. In some embodiments, a multivalent polypeptide of the disclosure includes an amino acid sequence that has 100% sequence identity to the amino acid sequence of SEQ ID NO: 34. In some embodiments, a multivalent polypeptide of the disclosure includes an amino acid sequence that has 100% sequence identity to the amino acid sequence of SEQ ID NO: 36. In some embodiments, a multivalent polypeptide of the disclosure includes an amino acid sequence that has 100% sequence identity to the amino acid sequence of SEQ ID NO: 38. In some embodiments, a multivalent polypeptide of the disclosure includes an amino acid sequence that has 100% sequence identity to the amino acid sequence of SEQ ID NO: 40. In some embodiments, a multivalent polypeptide of the disclosure includes an amino acid sequence that has 100% sequence identity to the amino acid sequence of SEQ ID NO: 42. In some embodiments, a multivalent polypeptide of the disclosure includes an amino acid sequence that has 100% sequence identity to the amino acid sequence of SEQ ID NO: 44.

In some particular embodiments, at least one of the first and the second polypeptide modules of the multivalent polypeptide of the present disclosure can include a multivalent antibody (e.g., bivalent antibody or trivalent antibody) including at least two antigen-binding moieties each possessing specific binding for a target protein. In some embodiments, the at least two antigen-binding moieties possess specific binding for the same target protein. Such antibody is multivalent, monospecific antibody. In some embodiments, the at least two antigen-binding moieties possessing specific binding for at least two different target proteins. Such antibody is multivalent, multispecific antibody (e.g., bispecific, trispecific, etc.) Accordingly, some embodiments disclosed herein relate to a multivalent antibody or functional fragment thereof, which includes (i) a first polypeptide module specific for one or more NKRs that signal through a phosphorylation mechanism, and (ii) a second polypeptide module specific for one or more RPTPs expressed on an immune cell that also expresses the NKRs, wherein the first polypeptide module is operably linked to the second polypeptide module. Accordingly, in some embodiments, at least one of the first and the second polypeptide modules of the disclosed multivalent antibody can be a bivalent, monospecific antibody. In some embodiments, at least one of the first and the second polypeptide modules of the disclosed multivalent antibody can be a trivalent, monospecific antibody. In some embodiments, at least one of the first and the second polypeptide modules of the disclosed multivalent antibody can be a bivalent, bispecific antibody. In some embodiments, at least one of the first and the second polypeptide modules of the disclosed multivalent antibody can be a trivalent, trispecific antibody.

One skilled in the art will appreciate that the complete amino acid sequence can be used to construct a back-translated gene. For example, a DNA oligomer containing a nucleotide sequence coding for a given polypeptide can be synthesized. For example, several small oligonucleotides coding for portions of the desired polypeptide can be synthesized and then ligated. The individual oligonucleotides typically contain 5′ or 3′ overhangs for complementary assembly.

In addition to generating multivalent polypeptides via expression of nucleic acid molecules that have been altered by recombinant molecular biological techniques, a subject multivalent polypeptide or multivalent antibody in accordance with the present disclosure can be chemically synthesized. Chemically synthesized polypeptides are routinely generated by those of skill in the art.

Once assembled (by synthesis, site-directed mutagenesis or another method), the DNA sequences encoding a multivalent polypeptide or multivalent antibody as disclosed herein will be inserted into an expression vector and operably linked to an expression control sequence appropriate for expression of the multivalent polypeptide or multivalent antibody in the desired transformed host. Proper assembly can be confirmed by nucleotide sequencing, restriction mapping, and expression of a biologically active polypeptide in a suitable host. As is known in the art, in order to obtain high expression levels of a transfected gene in a host, the gene must be operably linked to transcriptional and translational expression control sequences that are functional in the chosen expression host.

The binding activity of the multivalent polypeptides and multivalent antibodies of the disclosure can be assayed by any suitable method known in the art. For example, the binding activity of the multivalent polypeptides and multivalent antibodies of the disclosure can be determined by, e.g., Scatchard analysis (Munsen, et al. 1980 Analyt. Biochem. 107:220-239). Specific binding may be assessed using techniques known in the art including but not limited to competition ELISA, BIACORE® assays and/or KINEXA® assays. An antibody or polypeptide that “preferentially binds” or “specifically binds” (used interchangeably herein) to a target protein or target epitope is a term well understood in the art, and methods to determine such specific or preferential binding are also known in the art. An antibody or polypeptide is said to exhibit “specific binding” or “preferential binding” if it reacts or associates more frequently, more rapidly, with greater duration and/or with greater affinity with a particular protein or epitope than it does with alternative proteins or epitopes. An antibody or polypeptide “specifically binds” or “preferentially binds” to a target if it binds with greater affinity, avidity, more readily, and/or with greater duration than it binds to other substances. Also, an antibody or polypeptide “specifically binds” or “preferentially binds” to a target if it binds with greater affinity, avidity, more readily, and/or with greater duration to that target in a sample than it binds to other substances present in the sample. For example, an antibody or polypeptide that specifically or preferentially binds to a NKR epitope is an antibody or polypeptide that binds this epitope with greater affinity, avidity, more readily, and/or with greater duration than it binds to other NKR epitopes or non-NKR epitopes. It is also understood by reading this definition, for example, that an antibody or polypeptide (or moiety or epitope) which specifically or preferentially binds to a first target may or may not specifically or preferentially bind to a second target. As such, “specific binding” or “preferential binding” does not necessarily require (although it can include) exclusive binding.

A variety of assay formats may be used to select an antibody or polypeptide that specifically binds a molecule of interest. For example, solid-phase ELISA immunoassay, immunoprecipitation, Biacore™ (GE Healthcare, Piscataway, NJ), KinExA, fluorescence-activated cell sorting (FACS), Octet™ (ForteBio, Inc., Menlo Park, CA) and Western blot analysis are among many assays that may be used to identify an antibody that specifically reacts with an antigen or a receptor, or ligand binding portion thereof, that specifically binds with a cognate ligand or binding partner. Generally, a specific or selective reaction will be at least twice the background signal or noise, more typically more than 10 times background, even more typically, more than 50 times background, more typically, more than 100 times background, yet more typically, more than 500 times background, even more typically, more than 1000 times background, and even more typically, more than 10,000 times background. Also, an antibody is said to “specifically bind” an antigen when the equilibrium dissociation constant (K_{D}) is <7 nM.

The term “binding affinity” is herein used as a measure of the strength of a non-covalent interaction between two molecules, e.g., an antibody or portion thereof and an antigen. The term “binding affinity” is used to describe monovalent interactions (intrinsic activity). Binding affinity between two molecules may be quantified by determination of the dissociation constant (K_{D}). In turn, K_{D} can be determined by measurement of the kinetics of complex formation and dissociation using, e.g., the surface plasmon resonance (SPR) method (Biacore). The rate constants corresponding to the association and the dissociation of a monovalent complex are referred to as the association rate constants kₐ (or kₒₙ) and dissociation rate constant k_{d} (or k_{off}), respectively. K_{D} is related to kₐ and k_{d} through the equation K_{D}=k_{d}/kₐ. The value of the dissociation constant can be determined directly by well-known methods, and can be computed even for complex mixtures by methods such as those set forth in Caceci et al. (1984, Byte 9: 340-362). For example, the K_{D} may be established using a double-filter nitrocellulose filter binding assay such as that disclosed by Wong & Lohman (1993, Proc. Natl. Acad. Sci. USA 90: 5428-5432). Other standard assays to evaluate the binding ability of antibodies or polypeptides of the present disclosure towards target antigens are known in the art, including for example, ELISAs, Western blots, RIAs, and flow cytometry analysis, and other assays exemplified elsewhere herein. The binding kinetics and binding affinity of the antibody also can be assessed by standard assays known in the art, such as Surface Plasmon Resonance (SPR), e.g., by using a Biacore™ system, or KinExA.

### Nucleic Acids of the Disclosure

In one aspect, provided herein are various nucleic acid molecules including nucleotide sequences encoding the multivalent polypeptides and multivalent antibodies of the disclosure, including expression cassettes and expression vectors containing these nucleic acid molecules operably linked to regulator sequences which facilitate in vivo expression of the multivalent polypeptides and multivalent antibodies in a host cell or ex-vivo cell-free expression system.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The nomenclature for nucleotide bases as set forth in 37 CFR § 1.822 is used herein.

Nucleic acid molecules of the present disclosure can be nucleic acid molecules of any length, including nucleic acid molecules that are generally between about generally between about 0.5 Kb and about 20 Kb, for example between about 0.5 Kb and about 20 Kb, between about 1 Kb and about 15 Kb, between about 2 Kb and about 10 Kb, or between about 5 Kb and about 25 Kb, for example between about 10 Kb to 15 Kb, between about 15 Kb and about 20 Kb, between about 5 Kb and about 20 Kb, about 5 Kb and about 10 Kb, or about 10 Kb and about 25 Kb.

In some embodiments disclosed herein, the nucleic acid molecules of the disclosure include a nucleotide sequence encoding a multivalent polypeptide which include (a) a first amino acid sequence including a first polypeptide module capable of binding to a NK cell receptor (NKR) that signals through a phosphorylation mechanism; an (b) a second amino acid sequence including a second polypeptide module capable of binding to one or more receptor protein-tyrosine phosphatases (RPTPs) expressed on an immune cell that also expresses the NKR. In some embodiments, the nucleic acid molecules of the disclosure include a nucleotide sequence encoding a multivalent antibody which includes: (a) a first amino acid sequence including a first polypeptide module capable of binding to a NK cell receptor (NKR) that signals through a phosphorylation mechanism; an (b) a second amino acid sequence including a second polypeptide module capable of binding to one or more receptor protein-tyrosine phosphatases (RPTPs) expressed on an immune cell that also expresses the NKR.

In some embodiments disclosed herein, the nucleic acid molecules include a nucleotide sequence encoding a polypeptide that includes (i) an amino acid sequence having at least 80% sequence identity to the amino acid sequence of a multivalent polypeptide as disclosed herein or a functional fragment thereof; or (ii) an amino acid sequence having at least 80% sequence identity to the multivalent antibody of or a functional fragment thereof as disclosed herein. The nucleic acid molecules include a nucleotide sequence encoding a polypeptide that includes (i) an amino acid sequence having at least 90%, at least 95%, at least 96%, at least 97%, at least 98%, at least 99%, or 100% sequence identity to the amino acid sequence of a multivalent polypeptide as disclosed herein or a functional fragment thereof; or (ii) an amino acid sequence having at least 90%, at least 95%, at least 96%, at least 97%, at least 98%, at least 99%, or 100% sequence identity to the multivalent antibody of or a functional fragment thereof as disclosed herein.

In some embodiments, the nucleic acid molecules include a nucleotide sequence encoding a multivalent polypeptide which includes an amino acid sequence having at least 80%, at least 85%, at least 90%, at least 95%, at least 96%, at least 97%, at least 98%, at least 99%, or 100% sequence identity to an amino acid sequence selected from the group consisting of SEQ ID NOS: 1-2, 32, 34, 36, 38, 40, 42, and 44, or a functional fragment thereof. In some embodiments, the nucleic acid molecules include a nucleotide sequence encoding a multivalent polypeptide which includes an amino acid sequence having at least 80%, at least 85%, at least 90%, at least 95%, at least 96%, at least 97%, at least 98%, at least 99%, or 100% sequence identity to SEQ ID NO: 1, or a functional fragment thereof. In some embodiments, the nucleic acid molecules include a nucleotide sequence encoding a multivalent polypeptide which includes an amino acid sequence having at least 80%, at least 85%, at least 90%, at least 95%, at least 96%, at least 97%, at least 98%, at least 99%, or 100% sequence identity to SEQ ID NO: 2, or a functional fragment thereof. In some embodiments, the nucleic acid molecules include a nucleotide sequence encoding a multivalent polypeptide which includes an amino acid sequence having at least 80%, at least 85%, at least 90%, at least 95%, at least 96%, at least 97%, at least 98%, at least 99%, or 100% sequence identity to SEQ ID NO: 32, or a functional fragment thereof.

In some embodiments, the nucleic acid molecules include a nucleotide sequence encoding a multivalent polypeptide which includes an amino acid sequence having at least 80%, at least 85%, at least 90%, at least 95%, at least 96%, at least 97%, at least 98%, at least 99%, or 100% sequence identity to SEQ ID NO: 34, or a functional fragment thereof. In some embodiments, the nucleic acid molecules include a nucleotide sequence encoding a multivalent polypeptide which includes an amino acid sequence having at least 80%, at least 85%, at least 90%, at least 95%, at least 96%, at least 97%, at least 98%, at least 99%, or 100% sequence identity to SEQ ID NO: 36, or a functional fragment thereof. In some embodiments, the nucleic acid molecules include a nucleotide sequence encoding a multivalent polypeptide which includes an amino acid sequence having at least 80%, at least 85%, at least 90%, at least 95%, at least 96%, at least 97%, at least 98%, at least 99%, or 100% sequence identity to SEQ ID NO: 38, or a functional fragment thereof.

In some embodiments, the nucleic acid molecules include a nucleotide sequence encoding a multivalent polypeptide which includes an amino acid sequence having at least 80%, at least 85%, at least 90%, at least 95%, at least 96%, at least 97%, at least 98%, at least 99%, or 100% sequence identity to SEQ ID NO: 40, or a functional fragment thereof. In some embodiments, the nucleic acid molecules include a nucleotide sequence encoding a multivalent polypeptide which includes an amino acid sequence having at least 80%, at least 85%, at least 90%, at least 95%, at least 96%, at least 97%, at least 98%, at least 99%, or 100% sequence identity to SEQ ID NO: 42, or a functional fragment thereof. In some embodiments, the nucleic acid molecules include a nucleotide sequence encoding a multivalent polypeptide which includes an amino acid sequence having at least 80%, at least 85%, at least 90%, at least 95%, at least 96%, at least 97%, at least 98%, at least 99%, or 100% sequence identity to SEQ ID NO: 44, or a functional fragment thereof.

In some embodiments, the nucleic acid molecules include a nucleotide sequence that has at least 80%, at least 85%, at least 90%, at least 95%, at least 96%, at least 97%, at least 98%, at least 99%, or 100% sequence identity to a nucleotide sequence selected from the group consisting of SEQ ID NOS: 5-6 or a functional fragment thereof. In some embodiments, the nucleic acid molecules include a nucleotide sequence that has at least 80%, at least 85%, at least 90%, at least 95%, at least 96%, at least 97%, at least 98%, or at least 99% sequence identity to the nucleotide sequence of SEQ ID NO: 5, or a functional fragment thereof. In some embodiments, the nucleic acid molecules include a nucleotide sequence that has at least 80%, at least 85%, at least 90%, at least 95%, at least 96%, at least 97%, at least 98%, or at least 99% sequence identity to the nucleotide sequence of SEQ ID NO: 6, or a functional fragment thereof.

In some embodiments, the nucleic acid molecules include a nucleotide sequence that has 100% sequence identity to the nucleotide sequence of SEQ ID NO: 5, or a functional fragment thereof. In some embodiments, the nucleic acid molecules include a nucleotide sequence that has 100% sequence identity to the nucleotide sequence of SEQ ID NO: 6, or a functional fragment thereof.

In some embodiments, the recombinant nucleic acid molecules as disclosed herein can be incorporated into an expression cassette or an expression vector. Accordingly, some embodiments disclosed herein relate to vectors or expression cassettes including a recombinant nucleic acid molecule as disclosed herein. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into a subject. As such, in some embodiments, an expression cassette of the disclosure include a coding sequence for a multivalent polypeptide as disclosed herein, which is operably linked to expression control elements, such as a promoter, and optionally, any or a combination of other nucleic acid sequences that affect the transcription or translation of the coding sequence.

In some embodiments, the nucleic acid molecules of the disclosure can be incorporated into an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector. Accordingly, also provided herein are vectors, plasmids or viruses containing one or more of the nucleic acid molecules encoding any of the multivalent polypeptides and multivalent antibodies disclosed herein. The nucleic acid molecules described above can be contained within a vector that is capable of directing their expression in, for example, a cell that has been transduced with the vector. Suitable vectors for use in eukaryotic and prokaryotic cells are known in the art and are commercially available or readily prepared by a skilled artisan. Additional vectors can also be found, for example, in Ausubel, F. M., et al., Current Protocols in Molecular Biology, (Current Protocol, 1994) and Sambrook et al., “Molecular Cloning: A Laboratory Manual,” 2nd ED. (1989).

It should be understood that not all vectors and expression control sequences will function equally well to express the DNA sequences described herein. Neither will all hosts function equally well with the same expression system. However, one of skill in the art may make a selection among these vectors, expression control sequences and hosts without undue experimentation. For example, in selecting a vector, the host must be considered because the vector must replicate in it. The vector's copy number, the ability to control that copy number, and the expression of any other proteins encoded by the vector, such as antibiotic markers, should also be considered. For example, vectors that can be used include those that allow the DNA encoding the multivalent polypeptides and multivalent antibodies of the present disclosure to be amplified in copy number. Such amplifiable vectors are known in the art. They include, for example, vectors able to be amplified by DHFR amplification (see, e.g., Kaufman, U.S. Pat. No. 4,470,461) or glutamine synthetase (“GS”) amplification (see, e.g., U.S. Pat. No. 5,122,464 and European published application EP 338,841).

Accordingly, in some embodiments, the multivalent polypeptides and multivalent antibodies of the present disclosure can be expressed from vectors, generally expression vectors. The vectors are useful for autonomous replication in a host cell or may be integrated into the genome of a host cell upon introduction into the host cell, and thereby are replicated along with the host genome (e.g., non-episomal mammalian vectors). Expression vectors are capable of directing the expression of coding sequences to which they are operably linked. In general, expression vectors of utility in recombinant DNA techniques are often in the form of plasmids (vectors). However, other forms of expression vectors, such as viral vectors (e.g., replication defective retroviruses, adenoviruses, and adeno-associated viruses) are also included.

Exemplary recombinant expression vectors can include one or more regulatory sequences, selected on the basis of the host cells to be used for expression, operably linked to the nucleic acid sequence to be expressed.

DNA vector can be introduced into prokaryotic or eukaryotic cells via conventional transformation or transfection techniques. Suitable methods for transforming or transfecting host cells can be found in Sambrook et al. (1989) Molecular Cloning: A Laboratory Manual (2nd ed., Cold Spring Harbor Laboratory Press, Plainview, N.Y.) and other standard molecular biology laboratory manuals.

The nucleic acid sequences encoding the multivalent polypeptides and multivalent antibodies of the present disclosure can be optimized for expression in the host cell of interest. For example, the G-C content of the sequence can be adjusted to levels average for a given cellular host, as calculated by reference to known genes expressed in the host cell. Methods for codon optimization are known in the art. Codon usages within the coding sequence of the multivalent polypeptides and multivalent antibodies disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Vectors suitable for use include T7-based vectors for use in bacteria, the pMSXND expression vector for use in mammalian cells, and baculovirus-derived vectors for use in insect cells. In some embodiments nucleic acid inserts, which encode the subject multivalent polypeptide or multivalent antibody in such vectors, can be operably linked to a promoter, which is selected based on, for example, the cell type in which expression is sought.

In selecting an expression control sequence, a variety of factors should also be considered. These include, for example, the relative strength of the sequence, its controllability, and its compatibility with the actual DNA sequence encoding the subject multivalent polypeptide or multivalent antibody, particularly as regards potential secondary structures. Hosts should be selected by consideration of their compatibility with the chosen vector, the toxicity of the product coded for by the DNA sequences of this disclosure, their secretion characteristics, their ability to fold the polypeptides correctly, their fermentation or culture requirements, and the ease of purification of the products coded for by the DNA sequences.

Within these parameters one of skill in the art may select various vector/expression control sequence/host combinations that will express the desired DNA sequences on fermentation or in large scale animal culture, for example, using CHO cells or COS 7 cells.

The choice of expression control sequence and expression vector, in some embodiments, will depend upon the choice of host. A wide variety of expression host/vector combinations can be employed. Non-limiting examples of useful expression vectors for eukaryotic hosts, include, for example, vectors with expression control sequences from SV40, bovine papilloma virus, adenovirus and cytomegalovirus. Non-limiting examples of useful expression vectors for bacterial hosts include known bacterial plasmids, such as plasmids from E. coli, including col El, pCRI, pER32z, pMB9 and their derivatives, wider host range plasmids, such as RP4, phage DNAs, e.g., the numerous derivatives of phage lambda, e.g., NM989, and other DNA phages, such as M13 and filamentous single stranded DNA phages. Non-limiting examples of useful expression vectors for yeast cells include the 2μ plasmid and derivatives thereof. Non-limiting examples of useful vectors for insect cells include pVL 941 and pFastBac™ 1.

In addition, any of a wide variety of expression control sequences can be used in these vectors. Such useful expression control sequences include the expression control sequences associated with structural genes of the foregoing expression vectors. Examples of useful expression control sequences include, for example, the early and late promoters of SV40 or adenovirus, the lac system, the trp system, the TAC or TRC system, the major operator and promoter regions of phage lambda, for example PL, the control regions of fd coat protein, the promoter for 3-phosphoglycerate kinase or other glycolytic enzymes, the promoters of acid phosphatase, e.g., PhoA, the promoters of the yeast a-mating system, the polyhedron promoter of Baculovirus, and other sequences known to control the expression of genes of prokaryotic or eukaryotic cells or their viruses, and various combinations thereof.

A T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans will readily appreciate numerous promoters and other regulatory elements which can be used to direct expression of nucleic acids.

In addition to sequences that facilitate transcription of the inserted nucleic acid molecule, vectors can contain origins of replication, and other genes that encode a selectable marker. For example, the neomycin-resistance (neoR) gene imparts G418 resistance to cells in which it is expressed, and thus permits phenotypic selection of the transfected cells. Those of skill in the art can readily determine whether a given regulatory element or selectable marker is suitable for use in a particular experimental context.

Viral vectors that can be used in the disclosure include, for example, retroviral, adenoviral, and adeno-associated vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

In selecting an expression system, care should be taken to ensure that the components are compatible with one another. For example, an multivalent polypeptide or multivalent antibody as disclosed herein can be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

The expressed multivalent polypeptides or multivalent antibodies can be purified from the expression system using routine biochemical procedures, and can be used, e.g., as therapeutic agents, as described herein.

In some embodiments, multivalent polypeptides or multivalent antibodies obtained will be glycosylated or unglycosylated depending on the host organism used to produce the multivalent polypeptides or multivalent antibodies. If bacteria are chosen as the host then the multivalent polypeptide or multivalent antibody produced will be unglycosylated. Eukaryotic cells, on the other hand, will glycosylate the multivalent polypeptides or multivalent antibodies, although perhaps not in the same way as native polypeptides is glycosylated. The multivalent polypeptides or multivalent antibodies produced by the transformed host can be purified according to any suitable methods known in the art. Produced multivalent polypeptides or multivalent antibodies can be isolated from inclusion bodies generated in bacteria such as E. coli, or from conditioned medium from either mammalian or yeast cultures producing a given multivalent polypeptide or multivalent antibody using cation exchange, gel filtration, and or reverse phase liquid chromatography.

In addition or alternatively, another exemplary method of constructing a DNA sequence encoding the multivalent polypeptides or multivalent antibodies of the disclosure is by chemical synthesis. This includes direct synthesis of a peptide by chemical means of the protein sequence encoding for a multivalent polypeptide or multivalent antibody exhibiting the properties described. This method can incorporate both natural and unnatural amino acids at positions that affect the binding affinity of the multivalent polypeptide or multivalent antibody with the target protein. Alternatively, a gene which encodes the desired multivalent polypeptide or multivalent antibody can be synthesized by chemical means using an oligonucleotide synthesizer. Such oligonucleotides are designed based on the amino acid sequence of the desired multivalent polypeptide or multivalent antibody, and generally selecting those codons that are favored in the host cell in which the recombinant multivalent polypeptide or multivalent antibody will be produced. In this regard, it is well recognized in the art that the genetic code is degenerate—that an amino acid may be coded for by more than one codon. For example, Phe (F) is coded for by two codons, TIC or TTT, Tyr (Y) is coded for by TAC or TAT and his (H) is coded for by CAC or CAT. Trp (W) is coded for by a single codon, TGG. Accordingly, it will be appreciated by those skilled in the art that for a given DNA sequence encoding a particular multivalent polypeptide or multivalent antibody, there will be many DNA degenerate sequences that will code for that multivalent polypeptide or multivalent antibody. For example, it will be appreciated that in addition to the DNA sequences for multivalent polypeptides or multivalent antibodies provided in the Sequence Listing, there will be many degenerate DNA sequences that code for the multivalent polypeptides or multivalent antibodies disclosed herein. These degenerate DNA sequences are considered within the scope of this disclosure. Therefore, “degenerate variants thereof” in the context of this disclosure means all DNA sequences that code for and thereby enable expression of a particular multivalent polypeptide or multivalent antibody.

The DNA sequence encoding the subject multivalent polypeptide or multivalent antibody, whether prepared by site directed mutagenesis, chemical synthesis or other methods, can also include DNA sequences that encode a signal sequence. Such signal sequence, if present, should be one recognized by the cell chosen for expression of the multivalent polypeptide or multivalent antibody. It can be prokaryotic, eukaryotic or a combination of the two. In general, the inclusion of a signal sequence depends on whether it is desired to secrete the multivalent polypeptide or multivalent antibody as disclosed herein from the recombinant cells in which it is made. If the chosen cells are prokaryotic, the DNA sequence generally does not encode a signal sequence. If the chosen cells are eukaryotic, a signal sequence is generally included.

The nucleic acid molecules provided can contain naturally occurring sequences, or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., either a sense or an antisense strand).

The nucleic acid molecules are not limited to sequences that encode polypeptides; some or all of the non-coding sequences that lie upstream or downstream from a coding sequence (e.g., the coding sequence of a NKR, an anti-KIR2DL, an anti-NKG2A, or a NKR-RIPR molecule of the disclosure) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

Exemplary nucleic acid molecules of the present disclosure can include fragments not found as such in the natural state. Thus, this disclosure encompasses recombinant nucleic acid molecules, such as those in which a nucleic acid sequence (for example, a sequence encoding a NKR-RIPR molecule of the disclosure) is incorporated into a vector (e.g., a plasmid or viral vector) or into the genome of a heterologous cell (or the genome of a homologous cell, at a position other than the natural chromosomal location).

### Recombinant Cells and Cell Cultures

The multivalent polypeptides and recombinant nucleic acids of the present disclosure can be introduced into a cell, such as, for example, a eukaryotic cell, to produce a recombinant cell, e.g., an engineered cell. In some embodiments, the cell is in vivo. In some embodiments, the cell is ex vivo. In some embodiments, the cell is in vitro. In some embodiments, the recombinant cell is an animal cell. In some embodiments, the animal cell is a mammalian cell. In some embodiments, the animal cell is a human cell. In some embodiments, the cell is a non-human primate cell.

For example, a multivalent polypeptide and/or recombinant nucleic acid as disclosed herein can be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). In some embodiments, the recombinant cell is an immune cell. In some embodiments, the immune cell is a B cell, a monocyte, a natural killer (NK) cell, a natural killer T (NKT) cell, a basophil, an eosinophil, a neutrophil, a dendritic cell, a macrophage, a regulatory T cell, a helper T cell (T_{H}), a cytotoxic T cell (T_{CTL}), a memory T cell, a gamma delta (γδ) T cell, another T cell, a hematopoietic stem cell, or a hematopoietic stem cell progenitor. In some embodiments, the immune cell is a natural killer (NK) cell or a T cell.

In some embodiments, the immune cell is a NK cell. Non-limiting examples of NK cells suitable for the compositions and methods of the disclosure include NK-92 cells, taNK cells, aNKcells, haNK cells, and NKL cells. In some embodiments, the NK cell is a NK-92 cell. In some embodiments, the NK-92 cell is derived from an NK-92 cell line deposited with the American Type Culture Collection under accession number ATCC PTA-6672. In some embodiments, the NK-92 cell is derived from an NK-92 cell line deposited with the American Type Culture Collection under accession number ATCC CRL-2407. Additional NK cell lines suitable for the compositions and methods of the disclosure include, but are not limited to, NK cell lines MG4101, NK-92 MI, NK—S, NK-S7N, and NK YT-A1.

In some embodiments, the immune cell is a lymphocyte. In some embodiments, the lymphocyte is a T lymphocyte or a T lymphocyte progenitor that expresses one or more NKRs on its cell surface. Additional information in this regard can be found in, for example, Saligrama N. et al. (Nature, 2019 August; 572(7770): 481-487). In some embodiments, the T lymphocyte is a CD4+ T cell or a CD8+ T cell. In some embodiments, the T lymphocyte is a CD8+ T cytotoxic lymphocyte cell selected from the group consisting of naïve CD8+ T cells, CD8+ regulatory T cells, central memory CD8+ T cells, effector memory CD8+ T cells, effector CD8+ T cells, CD8+ stem memory T cells, and bulk CD8+ T cells. In some embodiments, the T lymphocyte is a CD4+ T helper lymphocyte cell selected from the group consisting of naïve CD4+ T cells, central memory CD4+ T cells, effector memory CD4+ T cells, effector CD4+ T cells, CD4+ stem memory T cells, and bulk CD4+ T cells.

In some embodiments, the immune cell expressing the NKR is a T cell, such as, e.g., a regulatory T cell (Treg cell). In some embodiments, the Treg cell is a CD8+ Treg cell as exemplified in Example 5, FIGS. 9A-9B and 10A-10B). As such, in some embodiments of the disclosure, the T cell is a CD8+ T cell expressing an NKR. In some embodiments, the NKR is a killer immunoglobulin-like receptor (KIR).

Accordingly, some embodiments of the disclosure relate to methods for making a recombinant cell, including (a) providing a host cell capable of protein expression; and transducing the provided host cell with a recombinant nucleic acid molecule of the disclosure to produce a recombinant cell. Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, a nucleic acid molecule of the disclosure can be introduced into a host cell by viral or non-viral delivery vehicles known in the art to produce a recombinant cell. For example, the nucleic acid molecule can be stably integrated in the recombinant cell's genome, or can be episomally replicating, or present in the recombinant cell as a mini-circle expression vector for transient expression. Accordingly, in some embodiments, the nucleic acid molecule is maintained and replicated in the recombinant host cell as an episomal unit. In some embodiments, the nucleic acid molecule is present in the recombinant cell as a mini-circle expression vector for transient expression. In some embodiments, the nucleic acid molecule is stably integrated into the genome of the recombinant cell. Stable integration can be achieved using classical random genomic recombination techniques or with more precise techniques such as guide RNA-directed CRISPR/Cas genome editing, or DNA-guided endonuclease genome editing with NgAgo (Natronobacterium gregoryi Argonaute), or TALENs genome editing (transcription activator-like effector nucleases).

The nucleic acid molecules of the disclosure can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, baculoviral virus or adeno-associated virus (AAV) can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g., transduced or transformed or transfected) with, for example, a vector construct of the present disclosure that can be, for example, a viral vector or a vector for homologous recombination that includes nucleic acid sequences homologous to a portion of the genome of the host cell, or can be an expression vector for the expression of the polypeptides of interest. Host cells can be either untransformed cells or cells that have already been transfected with at least one nucleic acid molecule.

In another aspect, provided herein are cell cultures including at least one recombinant cell as disclosed herein, and a culture medium. Generally, the culture medium can be any suitable culture medium for culturing the cells described herein. As discusses above, techniques for transforming a wide variety of the above-mentioned cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### Compositions and Pharmaceutical Compositions

In some embodiments, the multivalent polypeptides, multivalent antibodies, nucleic acids, recombinant cells, and cell cultures of the present disclosure can be incorporated into compositions, including pharmaceutical compositions. Such compositions generally include one or more the multivalent polypeptides, multivalent antibodies, nucleic acids, recombinant cells of the disclosure. In some embodiments, the compositions are pharmaceutical compositions. In some embodiments, the pharmaceutical compositions of the disclosure include a pharmaceutically acceptable excipient and one or more the following: multivalent polypeptides, multivalent antibodies, nucleic acids, recombinant cells of the disclosure.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.) or phosphate buffered saline (PBS). In some embodiments, care should be taken to ensure that the composition is sterile and is fluid to the extent that easy syringability exists. In some embodiments, care should also be taken to ensure that the composition is stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the common methods of preparation are vacuum drying and freeze-drying which yields a powder of the active ingredient plus any additional desired ingredient from a previously sterile-filtered solution thereof.

Oral compositions, if used, generally include an inert diluent or an edible carrier. For the purpose of oral therapeutic administration, the active compound (e.g., multivalent polypeptides, multivalent antibodies, nucleic acids, recombinant cells, and/or cell cultures of the disclosure) can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

In the event of administration by inhalation, the subject multivalent polypeptides, multivalent antibodies, nucleic acids, and/or recombinant cells of the disclosure are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration of the subject multivalent polypeptides, multivalent antibodies, nucleic acids, and recombinant cells of the disclosure can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, the multivalent polypeptides, multivalent antibodies, nucleic acids, and recombinant cells of the disclosure can also be prepared in the form of suppositories (e.g., with conventional suppository bases such as cocoa butter and other glycerides) or retention enemas for rectal delivery.

In some embodiments, the multivalent polypeptides, multivalent antibodies, nucleic acids, and recombinant cells of the disclosure can also be administered by transfection or infection using methods known in the art, including but not limited to the methods described in McCaffrey et al. (Nature 418:6893, 2002), Xia et al. (Nature Biotechnol. 20: 1006-1010, 2002), or Putnam (Am. J. Health Syst. Pharm. 53: 151-160, 1996, erratum at Am. J. Health Syst. Pharm. 53:325, 1996).

In some embodiments, the subject multivalent polypeptides, multivalent antibodies, nucleic acids, and recombinant cells of the disclosure are prepared with carriers that will protect the multivalent polypeptides, multivalent antibodies, nucleic acids, and recombinant cells against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

As described in greater detail below, the multivalent polypeptides and multivalent antibodies of the present disclosure may also be modified to achieve extended duration of action such as by PEGylation, acylation, Fc fusions, linkage to molecules such as albumin, etc. In some embodiments, the multivalent polypeptides or multivalent antibodies can be further modified to prolong their half-life in vivo and/or ex vivo. Non-limiting examples of known strategies and methodologies suitable for modifying the multivalent polypeptides or multivalent antibodies of the disclosure include (1) chemical modification of a multivalent polypeptide or multivalent antibody described herein with highly soluble macromolecules such as polyethylene glycol (“PEG”) which prevents the multivalent polypeptide or multivalent antibody from contacting with proteases; and (2) covalently linking or conjugating a multivalent polypeptide or multivalent antibody described herein with a stable protein such as, for example, albumin. Accordingly, in some embodiments, the multivalent polypeptide or multivalent antibody of the disclosure can be fused to a stable protein, such as, albumin. For example, human albumin is known as one of the most effective proteins for enhancing the stability of polypeptides fused thereto and there are many such fusion proteins reported.

In some embodiments, the pharmaceutical compositions of the disclosure include one or more pegylation reagents. As used herein, the term “PEGylation” refers to modifying a protein by covalently attaching polyethylene glycol (PEG) to the protein, with “PEGylated” referring to a protein having a PEG attached. A range of PEG, or PEG derivative sizes with optional ranges of from about 10,000 Daltons to about 40,000 Daltons may be attached to the multivalent polypeptides or multivalent antibodies of the disclosure using a variety of chemistries. In some embodiments, the pegylation reagent is selected from methoxy polyethylene glycol-succinimidyl propionate (mPEG-SPA), mPEG-succinimidyl butyrate (mPEG-SBA), mPEG-succinimidyl succinate (mPEG-SS), mPEG-succinimidyl carbonate (mPEG-SC), mPEG-Succinimidyl Glutarate (mPEG-SG), mPEG-N-hydroxyl-succinimide (mPEG-NHS), mPEG-tresylate and mPEG-aldehyde. In some embodiments, the pegylation reagent is polyethylene glycol; for example said pegylation reagent is polyethylene glycol with an average molecular weight of 20,000 Daltons covalently bound to the N-terminal methionine residue of the multivalent polypeptides and multivalent antibodies of the disclosure.

Accordingly, in some embodiments, a multivalent polypeptide or multivalent antibody of the disclosure are chemically modified with one or more polyethylene glycol moieties, e.g., PEGylated; or with similar modifications, e.g. PASylated. In some embodiments, the PEG molecule or PAS molecule is conjugated to one or more amino acid side chains of the multivalent polypeptide or multivalent antibody. In some embodiments, the PEGylated or PASylated multivalent polypeptide or multivalent antibody contains a PEG or PAS moiety on only one amino acid. In other embodiments, the PEGylated or PASylated multivalent polypeptide or multivalent antibody contains a PEG or PAS moiety on two or more amino acids, e.g., attached to two or more, five or more, ten or more, fifteen or more, or twenty or more different amino acid residues. In some embodiments, the PEG or PAS chain is 2000, greater than 2000, 5000, greater than 5,000, 10,000, greater than 10,000, greater than 10,000, 20,000, greater than 20,000, and 30,000 Da. The PASylated multivalent polypeptide or multivalent antibody may be coupled directly to PEG or PAS (e.g., without a linking group) through an amino group, a sulfhydryl group, a hydroxyl group, or a carboxyl group. In some embodiments, the multivalent polypeptide or multivalent antibody of the disclosure is covalently bound to a polyethylene glycol with an average molecular weight of 20,000 Daltons.

In some embodiments, the multivalent polypeptides or multivalent antibodies of the disclosure can be further modified to prolong their half-life in vivo and/or ex vivo. Non-limiting examples of known strategies and methodologies suitable for modifying the multivalent polypeptides or multivalent antibodies of the disclosure include (1) chemical modification of a multivalent polypeptide or multivalent antibody described herein with highly soluble macromolecules such as polyethylene glycol (“PEG”) which prevents the multivalent polypeptide or multivalent antibody from contacting with proteases; and (2) covalently linking or conjugating a multivalent polypeptide or multivalent antibody described herein with a stable protein such as, for example, albumin. Accordingly, in some embodiments, the multivalent polypeptide or multivalent antibody of the disclosure can be fused to a stable protein, such as, albumin. For example, human albumin is known as one of the most effective proteins for enhancing the stability of polypeptides fused thereto and there are many such fusion proteins reported.

### Methods of the Disclosure

Administration of any one of the therapeutic compositions described herein, e.g., multivalent polypeptides, multivalent antibodies, nucleic acids, recombinant cells, cell cultures, and pharmaceutical compositions, can be used in the treatment of relevant health conditions, such as proliferative diseases (e.g., cancers), autoimmune diseases, and chronic infections (e.g., viral infections). In some embodiments, the multivalent polypeptides, multivalent antibodies, nucleic acids, recombinant cells, cell cultures, and/or pharmaceutical compositions as described herein can be incorporated into therapeutic agents for use in methods of treating an individual who has, who is suspected of having, or who may be at high risk for developing one or more health conditions or diseases associated with cell signaling mediated by a NKR. Exemplary health conditions or diseases can include, without limitation, cancers and chronic infection. In some embodiments, the individual is a patient under the care of a physician.

Accordingly, in one aspect, some embodiments of the disclosure relate to methods for modulating cell signaling mediated by a NKR in a subject, the method includes administering to the subject a composition including one or more of: (i) a multivalent polypeptide of the disclosure, (ii) a multivalent antibody of the disclosure, (iii) a recombinant nucleic acid molecule of the disclosure, (iv) a recombinant cell of the disclosure, and (v) a pharmaceutical composition of the disclosure. In another aspect, some embodiments of the disclosure relate to methods for the treatment of a health condition in a subject in need thereof, the method including administering to the subject a composition including one or more of: (i) a multivalent polypeptide of the disclosure, (ii) a multivalent antibody of the disclosure, (iii) a recombinant nucleic acid molecule of the disclosure, (iv) a recombinant cell of the disclosure, and (v) a pharmaceutical composition of the disclosure. In some embodiments, the methods include administering a therapeutically effective amount of (i) a multivalent polypeptide of the disclosure, (ii) a multivalent antibody of the disclosure, (iii) a recombinant nucleic acid molecule of the disclosure, (iv) a recombinant cell of the disclosure, and/or (v) a pharmaceutical composition of the disclosure.

In some embodiments, the disclosed therapeutic composition is formulated to be compatible with its intended route of administration. For example, the multivalent polypeptides and multivalent antibodies of the disclosure may be given orally or by inhalation, but it is more likely that they will be administered through a parenteral route. Examples of parenteral routes of administration include, for example, intravenous, intradermal, subcutaneous, transdermal (topical), transmucosal, and rectal administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Dosage, toxicity and therapeutic efficacy of such subject multivalent polypeptides and multivalent antibodies of the disclosure can be determined by standard pharmaceutical procedures in cell cultures or experimental animals, e.g., for determining the LD50 (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are generally suitable. While compounds that exhibit toxic side effects may be used, care should be taken to design a delivery system that targets such compounds to the site of affected tissue in order to minimize potential damage to uninfected cells and, thereby, reduce side effects.

For example, the data obtained from the cell culture assays and animal studies can be used in formulating a range of dosage for use in humans. The dosage of such compounds lies generally within a range of circulating concentrations that include the ED₅₀ with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the IC₅₀ (e.g., the concentration of the test compound which achieves a half-maximal inhibition of symptoms) as determined in cell culture. Such information can be used to more accurately determine useful doses in humans. Levels in plasma may be measured, for example, by high performance liquid chromatography.

The therapeutic compositions described herein, e.g., multivalent polypeptides, multivalent antibodies, nucleic acids, recombinant cells, cell cultures, and pharmaceutical compositions, can be administered one from one or more times per day to one or more times per week; including once every other day. The skilled artisan will appreciate that certain factors may influence the dosage and timing required to effectively treat a subject, including but not limited to the severity of the disease, previous treatments, the general health and/or age of the subject, and other diseases present. Moreover, treatment of a subject with a therapeutically effective amount of the subject multivalent polypeptides and multivalent antibodies of the disclosure can include a single treatment or, can include a series of treatments. In some embodiments, the compositions are administered every 8 hours for five days, followed by a rest period of 2 to 14 days, e.g., 9 days, followed by an additional five days of administration every 8 hours. With regard to multivalent polypeptides or multivalent antibodies, the therapeutically effective amount of a multivalent polypeptide or multivalent antibody of the disclosure (e.g., an effective dosage) depends on the multivalent polypeptide or multivalent antibody selected. For instance, single dose amounts in the range of approximately 0.001 to 0.1 mg/kg of patient body weight can be administered; in some embodiments, about 0.005, 0.01, 0.05 mg/kg may be administered.

As discussed above, some embodiments of the disclosure relate to methods for modulating cell signaling mediated by a NKR in a subject. The method is performed by administering to the subject a composition including one or more of: (i) a multivalent polypeptide of the disclosure; (ii) a multivalent antibody of the disclosure; (iii) a recombinant nucleic acid molecule of the disclosure; (iv) a recombinant cell of the disclosure; and/or (v) a pharmaceutical composition of the disclosure. In another aspect, some embodiments of the disclosure relate to methods for the treatment of a health condition in a subject in need thereof. The method is performed by administering to the subject a composition including one or more of: (i) a multivalent polypeptide of the disclosure, (ii) a multivalent antibody of the disclosure, (iii) a recombinant nucleic acid molecule of the disclosure, (iv) a recombinant cell of the disclosure; and/or (v) a pharmaceutical composition of the disclosure. In some embodiments, the methods are performed by administering to the subject an effective amount of therapeutic composition as disclosed herein.

As discussed supra, a therapeutically effective amount includes an amount of a therapeutic composition that is sufficient to promote a particular effect when administered to a subject, such as one who has, is suspected of having, or is at risk for a health condition, e.g., a disease. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

The efficacy of a treatment including a disclosed therapeutic composition for the treatment of a health condition (e.g., disease) can be determined by the skilled clinician. However, a treatment is considered effective treatment if at least any one or all of the signs or symptoms of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

In some embodiments of the disclosed methods, the administered composition, e.g., multivalent polypeptide or multivalent antibody of the disclosure or nucleic acid encoding the same, recruits a RPTP activity into a spatial proximity of a NKR molecule present on the surface of a cell, eliciting phosphatase activity that reduces the phosphorylation level of the NKR molecule. In some embodiments, the administered multivalent polypeptide or multivalent antibody recruits the RPTP into a spatial proximity of a NKR molecule present on the surface of the same cell as the RPTP, e.g., the distance between the intracellular domain of the RPTP and the intracellular domain of the NKR molecule, in cis (e.g., the RPTP and the NKR molecule, are present in the same cell), is less than about 500 angstroms, such as e.g., a distance of about 5 angstroms to about 500 angstroms. In some embodiments, the spatial proximity amounts to less than about 5 angstroms, less than about 20 angstroms, less than about 50 angstroms, less than about 75 angstroms, less than about 100 angstroms, less than about 150 angstroms, less than about 250 angstroms, less than about 300 angstroms, less than about 350 angstroms, less than about 400 angstroms, less than about 450 angstroms, or less than about 500 angstroms. In some embodiments, the spatial proximity amounts to less than about 100 angstroms. In some embodiments, the spatial proximity amounts to less than about 50 angstroms. In some embodiments, the spatial proximity amounts to less than about 20 angstroms. In some embodiments, the spatial proximity amounts to less than about 10 angstroms. In some embodiments, the spatial proximity ranges from about 10 to 100 angstroms, from about 50 to 150 angstroms, from about 100 to 200 angstroms, from about 150 to 250 angstroms, from about 200 to 300 angstroms, from about 250 to 350 angstroms, from about 300 to 400 angstroms, from about 350 to 450 angstroms, or about 400 to 500 angstroms. In some embodiments, the administered multivalent polypeptide or the multivalent antibody recruits the RPTP into spatial proximity such that the RPTP is about 10 to 100 angstroms from the NKR molecule. In some embodiments, the spatial proximity amounts to less than about 100 angstroms. In some embodiments, the distance between the intracellular domain RPTP and the intracellular domain of the NKR molecule, in cis, is less than about 250 angstroms, alternatively less than about 200 angstroms, alternatively less than about 150 angstroms, alternatively less than about 120 angstroms, alternatively less than about 100 angstroms, alternatively less than about 80 angstroms, alternatively less than about 70 angstroms, or alternatively less than about 50 angstroms.

In some embodiments of the disclosed methods, the administered composition, e.g., multivalent polypeptide or multivalent antibody of the disclosure or nucleic acid encoding the same, recruits the RPTP activity to a spatial proximity of a NKR, potentiates dephosphorylation of a NKR, and/or reduces NKR-mediated signaling. In some embodiments, the administered composition confers an enhancement in NK cell killing of a target cell (e.g., killing of a target cell by an NK cell expressing an NK receptor.

In some embodiments, when the RPTP and the NKR molecule are brought into a spatial proximity of one to another, the RPTP potentiates dephosphorylation of the NKR molecule. In some embodiments, the phosphorylation level of the NKR molecule can be reduced by at least, or at least about, 10%, 15%, 20%, 25%, 30%, 35%, 40%, 45%, 50%, 55%, 60%, 65%, 70%, 75%, 80%, 85%, 90%, 95%, or 100%, or a range of any two of the proceeding values, for example from about 20% to about 60% (inclusive of values in between these percentages), as compared to the phosphorylation level of the NKR molecule in an untreated subject under similar conditions.

In some embodiments, the administration of a composition of the disclosure (e.g., multivalent polypeptide or multivalent antibody or nucleic acid encoding the same) confers an enhanced activity of NKR-mediated signaling in the subject. The reduction in activity of NKR-mediated signaling can be reduced by at least, or at least about, 10%, 15%, 20%, 25%, 30%, 35%, 40%, 45%, 50%, 55%, 60%, 65%, 70%, 75%, 80%, 85%, 90%, 95%, or 100%, or a range of any two of the proceeding values, for example from about 20% to about 60% (inclusive of values in between these percentages), as compared to the activity of NKR-mediated signaling in an untreated subject under similar conditions.

In some embodiments of the disclosed methods, the subject is a mammal. In some embodiments, the mammal is human. In some embodiments, the subject has or is suspected of having a health condition associated with inhibition of cell signaling mediated by a NKR. The health condition suitable for being treated by the compositions and methods of the disclosure include, but are not limited to, cancers, autoimmune diseases, inflammatory diseases, and infectious diseases. In some embodiments, the disease is a cancer or a chronic infection. In some embodiments, the health condition is an autoimmune condition such as, e.g., multiple sclerosis (MS), celiac disease, inflammatory bowel disease (IBD). In some embodiments, the health condition is an infectious disease where autoimmunity is a clinical problem.

### Additional Therapies

As discussed supra, any one of the compositions disclosed herein, e.g., multivalent polypeptides, multivalent antibodies, nucleic acids, recombinant cells, cell cultures, and/or pharmaceutical compositions described herein can be administered to a subject in need thereof as a single therapy (e.g., monotherapy). In addition or alternatively, in some embodiments of the disclosure, the multivalent polypeptides, multivalent antibodies, nucleic acids, recombinant cells, cell cultures, and/or pharmaceutical compositions described herein can be administered to the subject in combination with one or more additional therapies, e.g., at least one, two, three, four, or five additional therapies. Suitable therapies to be administered in combination with the compositions of the disclosure include, but are not limited to chemotherapy, radiotherapy, immunotherapy, hormonal therapy, toxin therapy, targeted therapy, and surgery. Other suitable therapies include therapeutic agents such as chemotherapeutics, anti-cancer agents, and anti-cancer therapies.

Administration “in combination with” one or more additional therapies includes simultaneous (concurrent) and consecutive administration in any order. In some embodiments, the one or more additional therapies is selected from the group consisting of chemotherapy, radiotherapy, immunotherapy, hormonal therapy, toxin therapy, and surgery. The term chemotherapy as used herein encompasses anti-cancer agents. Various classes of anti-cancer agents can be suitably used for the methods disclosed herein. Non-limiting examples of anti-cancer agents include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

Topoisomerase inhibitors are also another class of anti-cancer agents that can be used herein. Topoisomerases are essential enzymes that maintain the topology of DNA. Inhibition of type I or type II topoisomerases interferes with both transcription and replication of DNA by upsetting proper DNA supercoiling. Some type I topoisomerase inhibitors include camptothecins such as irinotecan and topotecan. Examples of type II inhibitors include amsacrine, etoposide, etoposide phosphate, and teniposide. These are semi synthetic derivatives of epipodophyllotoxins, alkaloids naturally occurring in the root of American Mayapple (Podophyllum peltatum).

Antineoplastics include the immunosuppressant dactinomycin, doxorubicin, epirubicin, bleomycin, mechlorethamine, cyclophosphamide, chlorambucil, ifosfamide. The antineoplastic compounds generally work by chemically modifying a cell's DNA.

Alkylating agents can alkylate many nucleophilic functional groups under conditions present in cells. Cisplatin and carboplatin, and oxaliplatin are alkylating agents. They impair cell function by forming covalent bonds with the amino, carboxyl, sulfhydryl, and phosphate groups in biologically important molecules.

Vinca alkaloids bind to specific sites on tubulin, inhibiting the assembly of tubulin into microtubules (M phase of the cell cycle). The vinca alkaloids include: vincristine, vinblastine, vinorelbine, and vindesine.

Anti-metabolites resemble purines (azathioprine, mercaptopurine) or pyrimidine and prevent these substances from becoming incorporated in to DNA during the “S” phase of the cell cycle, stopping normal development and division. Anti-metabolites also affect RNA synthesis.

Plant alkaloids and terpenoids are obtained from plants and block cell division by preventing microtubule function. Since microtubules are vital for cell division, without them, cell division cannot occur. The main examples are vinca alkaloids and taxanes.

Podophyllotoxin is a plant-derived compound which has been reported to help with digestion as well as used to produce two other cytostatic drugs, etoposide and teniposide. They prevent the cell from entering the G1 phase (the start of DNA replication) and the replication of DNA (the S phase).

Taxanes as a group includes paclitaxel and docetaxel. Paclitaxel is a natural product, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

In some embodiments, the anti-cancer agents can be selected from remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, gefitinib (Iressa®), taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, paclitaxel, vinblastine, IL-2, GM-CSF, dacarbazine, vinorelbine, zoledronic acid, palmitronate, biaxin, busulphan, prednisone, bortezomib (Velcade®), bisphosphonate, arsenic trioxide, vincristine, doxorubicin (Doxil®), paclitaxel, ganciclovir, adriamycin, estrainustine sodium phosphate (Emcyt®), sulindac, etoposide, and combinations of any thereof.

In other embodiments, the anti-cancer agent can be selected from bortezomib, cyclophosphamide, dexamethasone, doxorubicin, interferon-alpha, lenalidomide, melphalan, pegylated interferon-alpha, prednisone, thalidomide, or vincristine.

In some embodiments, the methods of treatment as described herein further include an immunotherapy. In some embodiments, the immunotherapy includes administration of one or more anti-NKR antagonistic molecules, such as anti-NKR antagonistic antibodies and functional variants thereof (e.g., anti-NKR blocking antibodies and functional variant thereof). In some embodiments, the antagonistic antibody is specific for a NKR expressed on the surface of an immune cell. Exemplary antagonistic antibodies suitable for the compositions and methods disclosed herein include chimeric antibody, monoclonal antibody, polyclonal antibody, recombinant antibody, Fab, Fab′, Fab₂, Fab′2, IgG, IgM, IgA, IgE, scFv, dsFv, dAb, nanobody, unibody, diabody, or hemibody. In some embodiments, the anti-NKR blocking antibody is a single chain variable fragment (scFv) or a Fab fragment specific for a NKR expressed on the surface of an immune cell. In some embodiments, the immunotherapy includes administration of one or more checkpoint inhibitors. Accordingly, some embodiments of the methods of treatment described herein include further administration of a compound that inhibits one or more immune checkpoint molecules. Non-limiting examples of immune checkpoint molecules include CTLA4, PD-1, PD-L1, A2AR, B7-H3, B7-H4, TIM3, and combinations of any thereof. In some embodiments, the compound that inhibits the one or more immune checkpoint molecules includes an antagonistic antibody. Examples of antagonistic antibodies suitable for the compositions and methods disclosed herein include, but are not limited to, ipilimumab, nivolumab, pembrolizumab, durvalumab, atezolizumab, tremelimumab, and avelumab.

In some aspects, the one or more anti-cancer therapy is radiation therapy. In some embodiments, the radiation therapy can include the administration of radiation to kill cancerous cells. Radiation interacts with molecules in the cell such as DNA to induce cell death. Radiation can also damage the cellular and nuclear membranes and other organelles. Depending on the radiation type, the mechanism of DNA damage may vary as does the relative biologic effectiveness. For example, heavy particles (i.e. protons, neutrons) damage DNA directly and have a greater relative biologic effectiveness. Electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray. Radiation also contemplated herein includes, for example, the directed delivery of radioisotopes to cancer cells. Other forms of DNA damaging factors are also contemplated herein such as microwaves and UV irradiation.

Radiation may be given in a single dose or in a series of small doses in a dose-fractionated schedule. The amount of radiation contemplated herein ranges from about 1 to about 100 Gy, including, for example, about 5 to about 80, about 10 to about 50 Gy, or about 10 Gy. The total dose may be applied in a fractioned regime. For example, the regime may include fractionated individual doses of 2 Gy. Dosage ranges for radioisotopes vary widely, and depends on the half-life of the isotope and the strength and type of radiation emitted. When the radiation includes use of radioactive isotopes, the isotope may be conjugated to a targeting agent, such as a therapeutic antibody, which carries the radionucleotide to the target tissue (e.g., tumor tissue).

Surgery described herein includes resection in which all or part of a cancerous tissue is physically removed, exercised, and/or destroyed. Tumor resection refers to physical removal of at least part of a tumor. In addition to tumor resection, treatment by surgery includes laser surgery, cryosurgery, electrosurgery, and microscopically controlled surgery (Mohs surgery). Removal of pre-cancers or normal tissues is also contemplated herein.

Accordingly, in some embodiments, the methods of the disclosure include administration of a composition disclosed herein to a subject individually as a single therapy (e.g., monotherapy). In some embodiments, a composition of the disclosure is administered to a subject as a first therapy in combination with a second therapy. In some embodiments, the second therapy is selected from the group consisting of chemotherapy, radiotherapy, immunotherapy, hormonal therapy, toxin therapy, and surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### Kits

Also provided herein are kits for the practice of a method described herein. A kit can include instructions for use thereof and one or more of the multivalent polypeptides, multivalent antibodies, nucleic acids, recombinant cells, and pharmaceutical compositions disclosed herein as described and provided herein. For examples, provided herein, in some embodiments, are kits that include one or more multivalent polypeptides and/or multivalent antibodies of the disclosure. In some embodiments, provided herein are kits that include one or more nucleic acids, recombinant cells, and/or pharmaceutical compositions of the disclosure. In some embodiments, the kits of disclosure further include written instructions for preparing the multivalent polypeptides, multivalent antibodies, nucleic acids, recombinant cells, and pharmaceutical compositions of the disclosure and using the same.

In some embodiments, the kits of the disclosure further include one or more syringes (including pre-filled syringes) and/or catheters (including pre-filled syringes) used to administer one any of the provided immune cells, nucleic acids, and pharmaceutical compositions to a subject in need thereof. In some embodiments, a kit can have one or more additional therapeutic agents that can be administered simultaneously or sequentially with the other kit components for a desired purpose, e.g., for modulating cell signaling mediated by one or more NKRs, or treating a health condition in a subject in need thereof.

For example, any of the above-described kits can further include one or more additional reagents, where such additional reagents can be selected from: dilution buffers; reconstitution solutions, wash buffers, control reagents, control expression vectors, negative control immune cell populations, positive control immune cell populations, reagents for ex vivo production of the immune cell populations. In some embodiments, any of the above-described kits can further include negative control polypeptides and/or antibodies, positive control polypeptides and/or antibodies, reagents for ex vivo and/or in vivo production of the polypeptides and/or antibodies.

In some embodiments, the components of a kit can be in separate containers. In some other embodiments, the components of a kit can be combined in a single container.

In some embodiments, a kit can further include instructions for using the components of the kit to practice the methods. The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferre, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

Additional embodiments are disclosed in further detail in the following examples, which are provided by way of illustration and are not in any way intended to limit the scope of this disclosure or the claims.

### Example 1

### General Experimental Procedures

### Cell Lines

Cell lines were kept in a humidified incubator at 37° C. with 5% CO₂ unless otherwise denoted.

HEK293T (LentiX) cells (female derived kidney cell line) were grown in DMEM complete media (Thermo Fisher) supplemented with 10% FBS, 2 mM L-glutamine, and 50 U/ml of penicillin and streptomycin. K562 and 721.221 cells were cultured in RPMI-1640 complete media containing 10% FBS, 2 mM L-glutamine, 0.1 mM NEAA, 1 mM sodium pyruvate, 10 mM HEPES, 50 U/ml P/S, 50 μg/ml gentamycin sulfate.

NK92 cells. NK92 cells used in the experiments were purchased from the American Type Culture Collection and derived from a NK lymphoma cell line which has bene established from the peripheral blood of a patient with malignant non-Hodgkin's lymphoma. NK92 cells were cultured in RPMI 1640 medium supplemented with 2 mM L-glutamine, 0.1 mM NEAA, 1 mM sodium pyruvate, 10 mM HEPES, 50 U/ml P/S and 10% fetal bovine serum

NKL-KIR2DL cell lines. NKL cells used in these experiments were from a natural killer cell lymphoblastic leukemia cell line, which has been established from the peripheral blood of a patient with lymphoblastic leukemia. The NKL cells was transduced with a retroviral vector carrying a KIR2DL coding sequence to produce NKL-KIR2DL cells. The NKL-KIR2DL cells were cultured in RPMI 1640 medium supplemented with 2 mM L-glutamine, 0.1 mM NEAA, 1 mM sodium pyruvate, 10 mM HEPES, 50 U/ml P/S and 10% fetal bovine serum.

SKW3 cells used in the experiments described herein were from a T cell leukemia cell line (CLL), which has been established from the blood of a patient with chronic lymphocytic leukemia (CLL). SKW3 cells were grown in RPMI 1640 medium supplemented with 2 mM L-glutamine, 0.1 mM NEAA, 1 mM sodium pyruvate, 10 mM HEPES, 50 U/ml P/S and 10% fetal bovine serum. When needed, SKW3 cultures were subcultured by addition or replacement of fresh medium. Establish new cultures at 2×10⁵ viable cells/ml. Maximum cell density at 2×10⁶ cell s/ml.

### Protein Expression

Insect Tni cells (Expression Systems, cat. #94-002S) were grown in ESF 921 media (Expression Systems) with a final concentration of 10 mg/L of gentamicin sulfate (Thermo Fisher) at 27° C. and atmospheric CO₂. SF9 cells (Thermo Fisher Scientific) were grown in SF900-III or -II serum-free media (Thermo Fisher) with 10% FBS and final concentration 10 mg/L of gentamicin sulfate and 2 mM Glutamax at 27° C. and atmospheric CO₂. P1 virus was used to infect volumes of 1-3 L of cells at ˜2×10⁶ cells/ml. New P1 preps were made from fresh P0 batches routinely. Supernatant was harvested 2-3 days post-infection and spun down at 8000 rpm for 15 minutes. The supernatant containing expressed protein was treated to 100 mM Tris pH 8.0, 2 mM NiCl₂, and 10 mM CaCl₂) to precipitate contaminants. The supernatant and precipitate mixture was spun down at 8000 rpm for 20 min at 4° C. to remove precipitate. The supernatant was incubated with Ni-NTA resin (QIAGEN) for >3 hours at room temperature. Ni-NTA beads were collected and washed in a Buchner funnel with 20 mM imidazole in 1×EMS pH 7.2 and eluted with 200 mM imidazole in 1×EMS pH 7.2. Protein was concentrated in a 10 kDa filter (Millipore, UFC903024) to ˜1 mL or until 10 mg/ml. All proteins were further purified by size-exclusion chromatography using Superdex Increase S200 or S75, as appropriate (GE Healthcare). All proteins for in vivo studies were cleared of endotoxin. Final endotoxin levels were determined using a chromogenic endotoxin quantitation kit (Thermo Fisher) and never greater than 1 Endotoxin Unit/mg of purified protein. RIPR proteins were kept at 4° C. for up to two weeks to prevent freeze/thawing cycle.

### Protein Integrity and Stability Analyzed by Size-Exclusion Chromatography

Proteins were concentrated, filtered by 0.45 μm centrifugation filters, and subsequently loaded on 2-mL injection loop of an 5200 Increase or a S75 column (GE Healthcare), depending on protein size, on an AKTA Pure FPLC (GE Healthcare). All proteins were eluted in 1×HBS (30 mM HEPES pH 7.2, 150 mM NaCl). Following elution, a sample from each protein fraction was run on a 4%-20% Mini-PROTEAN gel and gels were Coomassie stained to check for protein degradation and identify fractions containing intact protein for pooling of samples.

### Surface Expression Quantified by Flow Cytometry

1×10⁶ live cells were washed with FACS buffer (PBS, 0.5% FBS, 0.9% sodium azide) and incubated with Fc block for 10 minutes at 4° C. The cells were washed and stained with antibodies or isotype controls for 20 minutes at 4° C. After washing twice, the cells were resuspended in 1 μg/ml DAPI (PBS) solution and analyzed on CytoFlex flow cytometer. Data were analyzed and quantified using Flowjo software.

### Example 2

### NKR-RIPR Designs and Expression

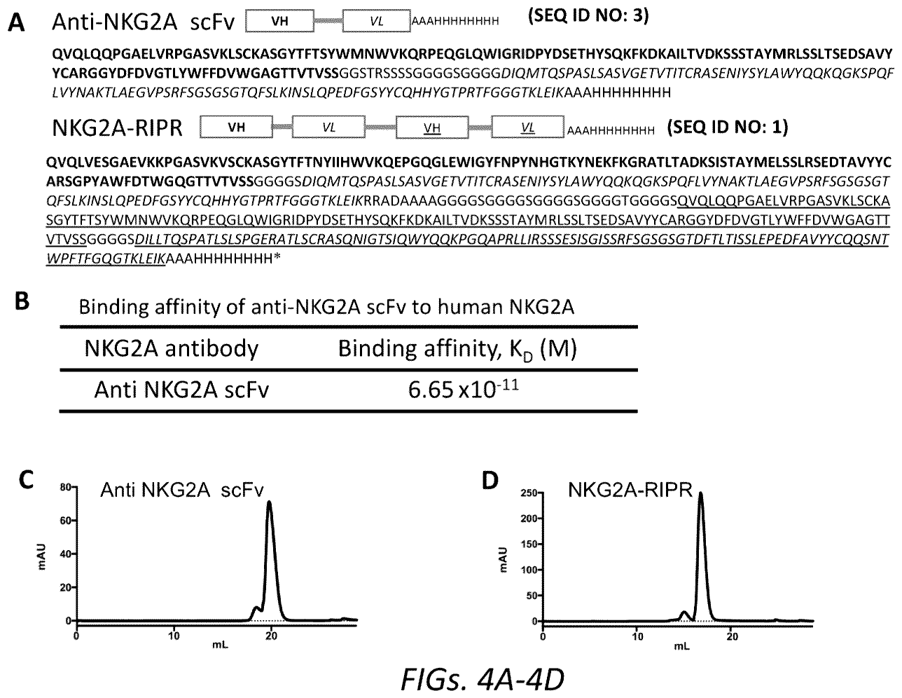

Two NKR-RIPR molecules were developed. First construct, NKG2A-RIPR, was composed from an anti-CD45 scFv (clone #4, as described previously in WO2005/026210) fused to a murine anti-NKG2A scFv having binding affinity for a human NKG2A. The anti-NKG2A scFv used in this experiment was derived from the V_{H} and V_{L} sequences of the murine antibody Z270 described previously in U.S. Pat. No. 9,683,041. A schematic depiction of anti-NKG2A scFv and NKG2A-RIPR molecule design is shown in FIG. 2B. The results of experiments performed to evaluate binding affinity of anti-NKG2A scFv to human NKG2A are shown in FIG. 4B. Anti-NKG2A scFv and NKG2A-RIPR were expressed in Hi5 cells and protein integrity and stability was analyzed by size-exclusion chromatography (see, e.g., FIG. 4C).

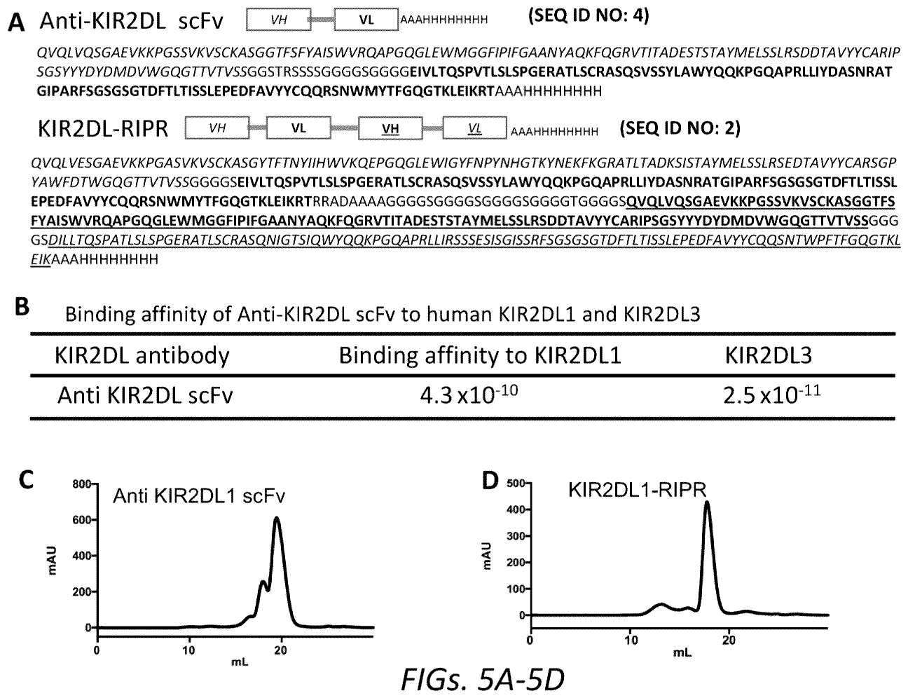

The second construct, KIR2DL-RIPR was composed from the same anti-CD45 scFv fused to a human anti-KIR2DL scFv having binding affinity for a human KIR2DL1 and/or human KIR2DL3. The anti-KIR2DL scFv used in this experiment was derived from the V_{H} and VL sequences of the human antibody 1-7F9 described previously in EP2287195A2. A schematic depiction of αKIR2DL scFv and KIR2DL-RIPR molecule design is shown in FIG. 3B. The results of experiments performed to evaluate binding affinity of anti-KIR2DL scFv to human KIR2DL1 and human KIR2DL3 are shown in FIG. 5B. Anti-KIR2DL scFv and KIR2DL-RIPR were expressed in Hi5 cells and protein integrity and stability was analyzed by size-exclusion chromatography (see, e.g., FIGS. 5C-5D).

All proteins were purified using Ni-NTA and the fractions corresponding to a monodisperse peak after SEC were pooled and concentrated. Protein integrity was further confirmed by reduced and non-reducing SDS-PAGE electrophoresis followed by Coomassie blue staining. Protein was kept at 4° C. for immediate use or stored frozen at −80° C.

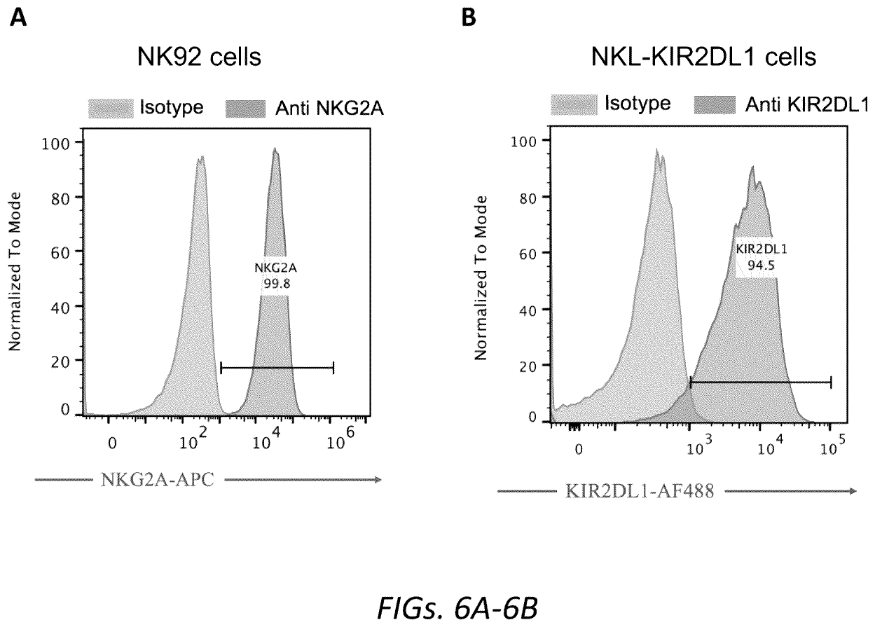

FIGS. 6A-6B summarize the results of experiments performed to illustrate the surface expression of NKG2A on NK92 cells (FIG. 6A) and KIR2DL1 on NKL-KIR2DL1 cells. (FIG. 6B). In these experiments, surface expression was quantified by flow cytometry.

### Example 3

### NKG2A-RIPR Potentiates Dephosphorylation of Human NKG2A and Enhances Lysis of Target Cell

This Example describes experiments performed to illustrate that NKG2A-RIPR potentiates dephosphorylation of human NKG2A and enhances lysis of target cell.

As shown in FIG. 7A, to reconstitute the NKG2A phosphorylation, approximately 4×10⁶ HEK293 cells were transiently transfected with plasmids encoding full length human Lck, CD45, CD45_{dead}, or NKG2A at an optimized ratio. 24 hours after transfection, cells were left untreated (lane 1) or incubated for 20 min at 37° C. with anti-NKG2A scFv (lane 2 and 3) or NKG2A-RIPR (lane 4, 5) to induce recruitment of the CD45 phosphatase to the intracellular domains of NKG2A. A CD45_{dead} group was included for control purposes. After lysis, proteins were immunoprecipitated with anti-HA antibody directly conjugated to magnetic beads. Samples were probed for phosphotyrosine (pTyr) and HA by western blot. Data are representative of three independent biological repeats.

### Example 4

### KIR2DL-RIPR Enhances Lysis of Target Cell

This Example describes experiments performed to illustrate that KIR2DL-RIPR enhances lysis of target cell.

Without being bound to any particular theory, FIG. 8A graphically illustrates a non-limiting example of the modulation of cellular KIR2DL-mediated signaling by in cis phosphatase recruitment in accordance with some embodiments of the disclosure. The recruitment of CD45 to KIR2DL1 by NKR-RIPR at the cell surface of NK cells is anticipated to decrease phosphorylation of the receptor KIR2DL1.

As shown in FIG. 8B, KIR2DL-RIPR enhances lysis of target cell. In these experiments, cell lysis by NKL cells that express KIR2DL1 against HLA-Cw0304 positive 721.221 cells in the presence of 200 nM (top) or 1000 nM (bottom) anti-KIR2DL scFv or KIR2DL-RIPR. Cell lysis was determined by flow cytometry.

### Example 5

### NKR-RIPR Inhibits NKR Receptors Expressed on CD8+ T Cells and Enhances T Cells Functionality

This Example describes experiments performed to illustrate an enhancement of TCR signaling in CD8+ T cells expressing a NK receptor (KIR2DL1) by an anti-KIR2DL1 antagonistic antibody or by a KIR2DL-RIPR construct in accordance with some embodiments of the disclosure.

These experiments were performed with CD8+ regulatory T cells genetically engineered to express at least one NK receptor. Without being bound to any particular theory, CD8+ Treg cells were believed to be important in autoimmune diseases and thought to suppress the actions of CD4+ pathogenic autoimmune T cells. The experiments described in this Example were designed to investigate if the expression of inhibitory NK receptors on CD8+ T cells would result in inhibition of T cell activation.

In these experiments, KIR2DL1 was lentivirally infected into CD8+SKW3 cells expressing a T cell receptor (TCR55). Transduced cells were subsequently sorted for stable co-expression of TCR55 and KIR2DL1. Surface expression of CD8 and KIR2DL1 in CD8+SKW3-TCR55 cells was then quantified by flow cytometry (as shown in FIGS. 9A and 9B).

Additional experiments were performed to investigate whether inhibition of NKR activity in NKR+CD8 T cells by anti-NKR and/or NKR-RIPR could enhance of TCR signaling in the NKR+CD8 T cells. As shown in FIG. 10A, it was observed that expression of the receptor KIR2DL1 on CD8+ T cells suppressed T cell activation by peptide-WIC. In these experiments, approximately 1×10⁴ 721.221 antigen-presenting cells expressing HLA-B35 and HLA-Cw0304 were pulsed with HIV peptide 20 for 3 hours at 37° C., then co-incubated with SKW3 KIR2DL1(+) or SKW3 KIR2DL1(−) cell line at 1:1 ratio for 16 hours. The response effect of HIV peptide was analyzed for CD69 expression by flow cytometry.

In addition, as shown in FIG. 10B, blockade of KIR2DL1 by an anti-KIR2DL scFv partially reversed inhibition of T cell activation (green trace). Furthermore, a KIR2DL-RIPR construct was found to nearly completely restore full CD8+ T cell activation (red trace) and with a superior efficacy compared to NKR inhibition by anti-KIR2DL scFv. In these experiments, HLA-B35/Cw0304 721.221 APC cells are pulsed with HIV peptide 20 for 3 hours, then co-incubated with SKW3 KIR2DL1(+) cell line in the presence 200 nM anti-KIR2DL scFv or KIR2DL-RIPR for 16 hours. CD69 activation of SKW3 T cells were tested.

Without being bound to any particular theory, since expression of NK receptors on CD8+NKR+ T cells results in a reduced T cell signaling, it is contemplated that a therapeutic strategy to “de-repress” these T cells can be useful in inhibiting NKR activity in these cells. The experimental results described in this Example have demonstrated that inhibition of KIR2DL or other NK receptors on CD8+ T cells enhances the T cell activity and could be useful as a therapeutic strategy for treatment of autoimmune diseases. Importantly, the experimental results described herein have demonstrated that both simple NKR blockade approach with an antagonistic antibody and NKR-RIPR mediated inhibition approach (with superior efficacy compared to blockade) could be therapeutic strategies for autoimmune conditions such as, e.g., multiple sclerosis (MS), celiac disease, inflammatory bowel disease (IBD), as well as infectious diseases where autoimmunity is a clinical problem.

### Example 6

### NKG2A-RIPR Potentiates Activation of NK and CD8+ T Cells and Ly49-RIPR Enhances NK Killing

This Example describes experiments performed to illustrate potentiation of target lysis by three exemplary constructs, NKG2A-RIPR, Ly49C/I-RIPR, and KIR-RIPR, in accordance with some embodiments of the disclosure. The sequence of Ly49 used in this experiment corresponds to NCBI accession number PMID: 31676749. The NCBI accession numbers for Ly49C and Ly49I are NM_001289604, NM_010651.

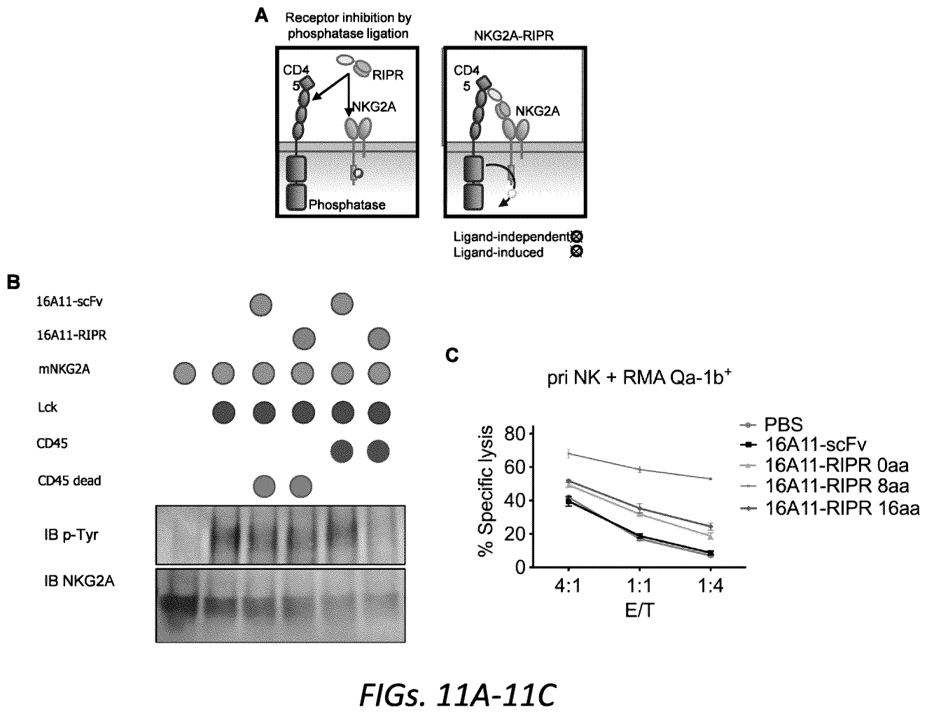

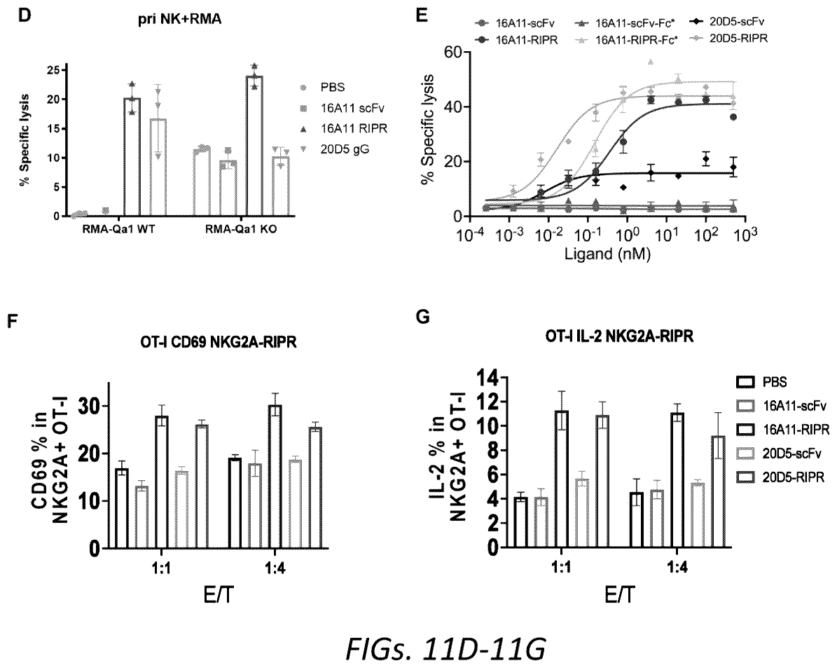

As shown in FIGS. 11A-11G, NKG2A-RIPR potentiates activation of NK and CD8+ cells. The binding of an NKG2A-RIPR construct to both CD45 and NKG2A by NKG2A-RIPR results in recruitment of CD45 phosphatase to NKG2A, in cis, on the surface of a cell (e.g., NK cell or T cell). In these experiments, HEK293 cells were transiently transfected with mouse HA-NKG2A, Lck, and mouse CD45. Twenty-four hours after transfection, as shown in FIG. 11B, cells were left untreated (lane 1) or incubated for 40 minutes at 37° C. with 16A11 scFv (lane 5) or 16A11-RIPR (lane 4) to induce recruitment of the CD45 phosphatase to the intracellular domains of NKG2A. A CD45_{dead} group (Lane 3, 4) was included for control purposes. As described above, CD45_{dead} is a CD45 variant with a mutation that causes loss of CD45 phosphatase activity. 16A11 had no detectable effect on reducing phosphorylation of NKG2A, probably because 16A11 is a non-blocking antibody. Compared to 16A11, the 16A11-RIPR construct was found to significantly potentiate dephosphorylation of mouse NKG2A.

Further experiments were performed to optimize the length of the linker inserted between 16A11 scFv and CD45 V_{H}H sequences in the 16A11-RIPR constructs. Ex vivo expanded mouse NKG2A+NK cells were co-cultured with target cells at different ratios. IFNγ pre-treated RMA, which is a cell line expressing Qa-1, a natural ligand of NKG2A was used as target cells for NK killing. 100 nM 16A11-scFv, 16A11-RIPR-0aa, 16A11-RIPR-8aa and 16A11-RIPR-16aa were added in the co-culture wells. Four hours later, the NK cytotoxicity was analyzed by annexin V/7AAD assay. The results are summarized in FIG. 11C. After testing a panel of 16A11-RIPR with different linker length, it was observed that 16A11-RIPR-8aa could best induce NK activity among these tested molecules.

Experimental results presented in FIG. 11D demonstrate the biased effect of 16A11-RIPR and 20D5 in NK cytotoxicity against Qa1(+) and Qa-1(−) cells. 20D5 is a monoclonal antibody against mouse NKG2A. IFNγ pre-treated RMA was used as target cells for NK killing. Ex vivo expanded mouse NKG2A+NK cells were co-cultured with target cells (RMA-Qa1 WT or RMA-Qa1 KO cells) at different ratios. 100 nM 16A11-scFv, 16A11-RIPR, and 10 ug/mL 20D5-IgG were added in the co-culture wells. Four hours later the NK cytotoxicity was analyzed by annexin V/7AAD assay. In these experiments, by comparing the NK killing effect of 16A11-RIPR with 20D5-IgG against two RMA cell lines, it was observed that 16A11-RIPR potentiates NK killing against both RMA-Qa1 WT and RMA-Qa1 KO cells. However, 20D5-IgG preferentially induces NK killing effect on RMA-Qa1 WT cells. The experimental results presented herein demonstrates a biased effect of 16A11-RIPR and 20D5 in NK cytotoxicity against RMA-Qa1 WT and RMA-Qa1 KO cells.

Additional experiments were performed to evaluate the effectiveness of various RIPR constructs in targeting cells of target cells for NK killing. As shown in FIG. 11E, 20D5-RIPR was found to be superior to 16A11-RIPR in NK killing of target cells. Fusion of a mouse Fc retained full activity of 16A11-RIPR in NK killing. The Fc region used in these experiments were derived from mouse IgG1 with an N279Q mutation, which lacks glycosylation at N297 and does not bind mFcγR. In these experiments, IFNγ pre-treated RMA was used as target cells for NK killing. Ex vivo expanded mouse NKG2A+NK cells were cocultured with target cells at 1:2 ratio. 16A11-RIPR, scFv control, 20D5-RIPR, 20D5-scFv in dose series were added in the coculture wells. After four hours, the NK cytotoxicity was analyzed by annexin V/7AAD assay. Dose-response curves in FIG. 11E show mean±standard deviation of duplicate wells. In these experiments, by fusing a half-life extender to the C terminus of 16A11-RIPR, it was observed that the Fc fused 16A11-RIPR remained fully active. Although 20D5-RIPR displayed a lower EC50 than 16A11-RIPR, both constructs were found to induce similar NK killing.

Experimental results presented in FIGS. 11F and 11G demonstrate that NKG2A-RIPRs potentiate CD8+OT1 activation. In these experiments, B16-OVA cells were used as target cells. Ex vivo expanded mouse NKG2A+OT1 cells were co-cultured with target cells at different ratios. 100 nM 16A11-RIPR, 16A11-scFv, 20D5-RIPR, 20D5-scFv were added in the co-culture wells. After four hours, the OT1 activation was analyzed by surface staining of CD69 and intracellular staining of IL-2. NKG2A was previously reported to be able to be upregulated in CD8+ T cells. Experimental results described in this Example demonstrate that both 16A11-RIPR and 20D5-RIPR can enhance the activity of CD8+ T cells.

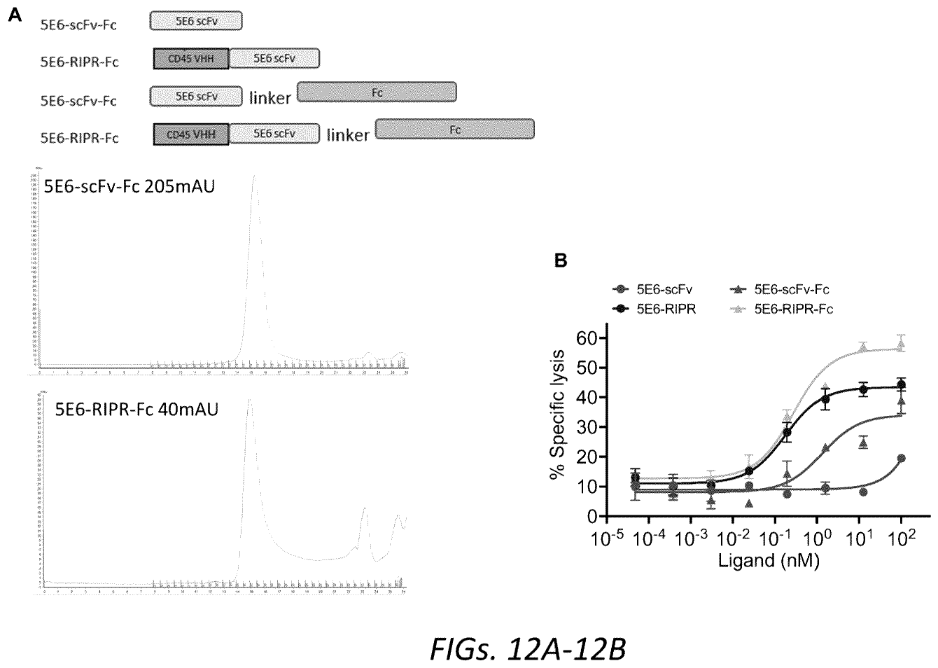

As shown in FIGS. 12A-12B, the fusion of Fc potentiates the NK activity of both 5E6-scFv and 5E6-RIPR. Design and expression of anti-Ly49C/I scFv (clone 5E6), 5E6-RIPR, 5E6-scFv-Fc, and 5E6-RIPR-Fc are shown in FIG. 12A. FIG. 12B shows that fusion of Fc potentiates the NK activity of both 5E6-scFv and 5E6-RIPR. IFNγ pre-treated RMA was used as target cells for NK killing. Ex vivo expanded mouse Ly49C/I+NK cells were co-cultured with target cells at 1:2 ratio. 5E6-RIPR, scFv control, 5E6-RIPR-Fc, 5E6-scFv-Fc were added in the co-culture wells. Four hours later the NK cytotoxicity was analyzed by annexin V/7AAD assay. Dose-response curves show mean±standard deviation of duplicate wells. Consistent with previous reports, it was observed that 5E6 scFv functions as a blocking antibody. Tt was observed that (i) 5E6-RIPR enhances the NK killing effect, and (ii) Fc fused 5E6-RIPR shows activity beyond 5E6-RIPR.

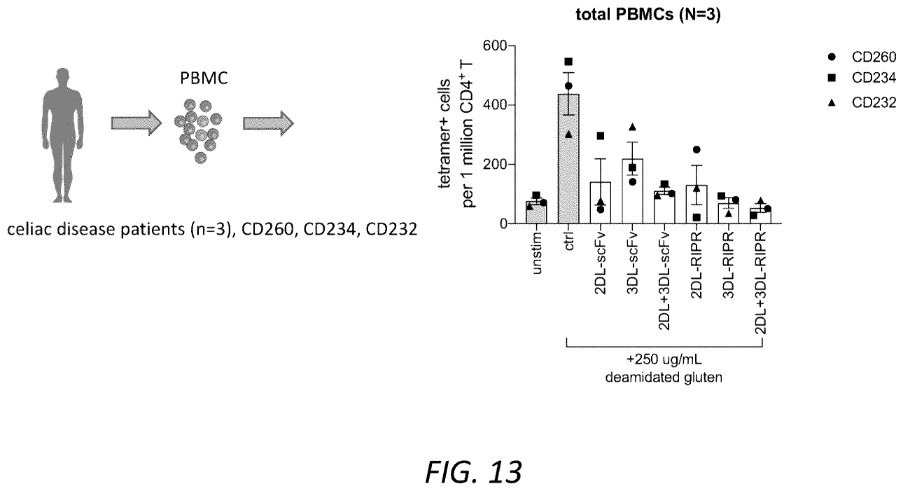

FIG. 13 shows that KIR3DL-RIPR potentiates the elimination of gliadin-specific CD4+ T cells by KIR+CD8+ T cells. Total peripheral blood mononuclear cells (PBMCs) were harvested from 3 donors having celiac disease. The cells were immunized with gluten and treated with KIR2DL scFv, KIR3DL scFv, KIR2DL-RIPR, KIR3DL-RIPR, 2DL+3DL scFv, 2DL+3DL-RIPR. The number of gliadin-specific CD4+ T cells (tetramer positive cells) in CD4 T cells were quantified by fluorescence assisted cell sorting (FACS). Experimental results described in this Example indicate that KIR-RIPR could be applied to celiac disease treatment by activating CD8+ Treg cells.

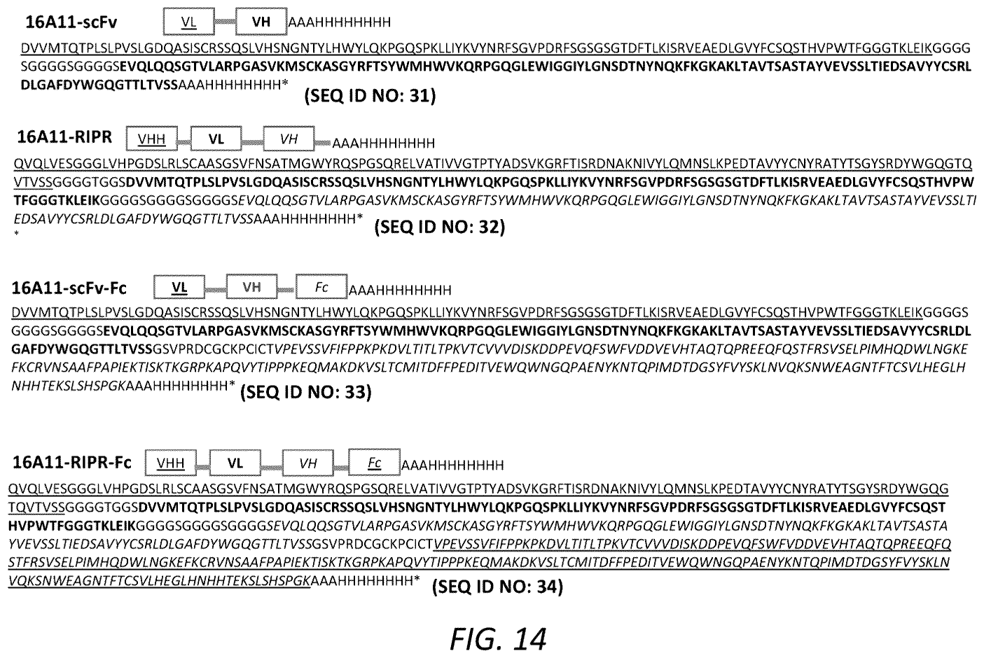

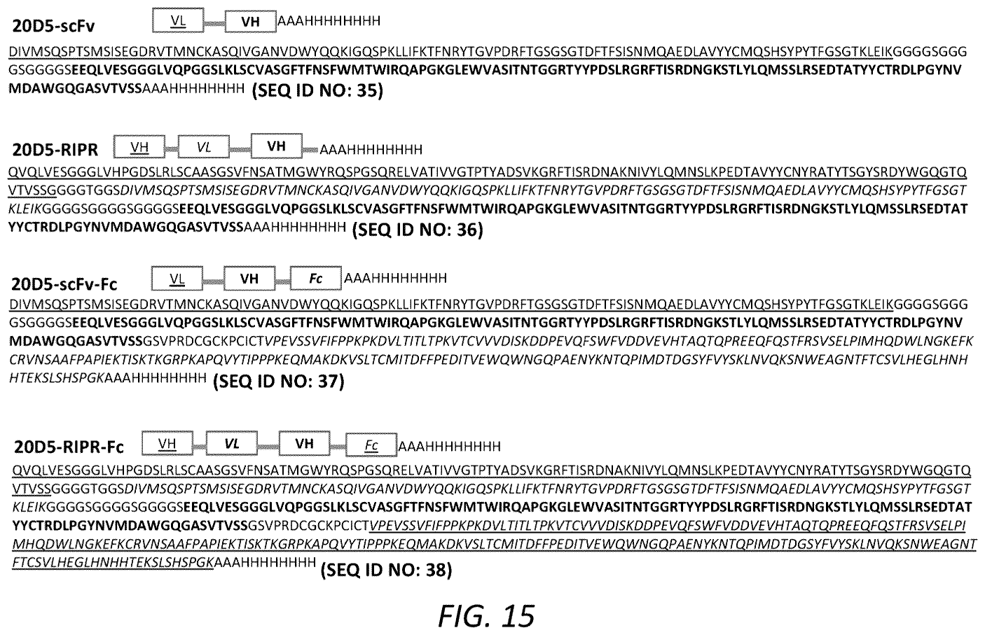

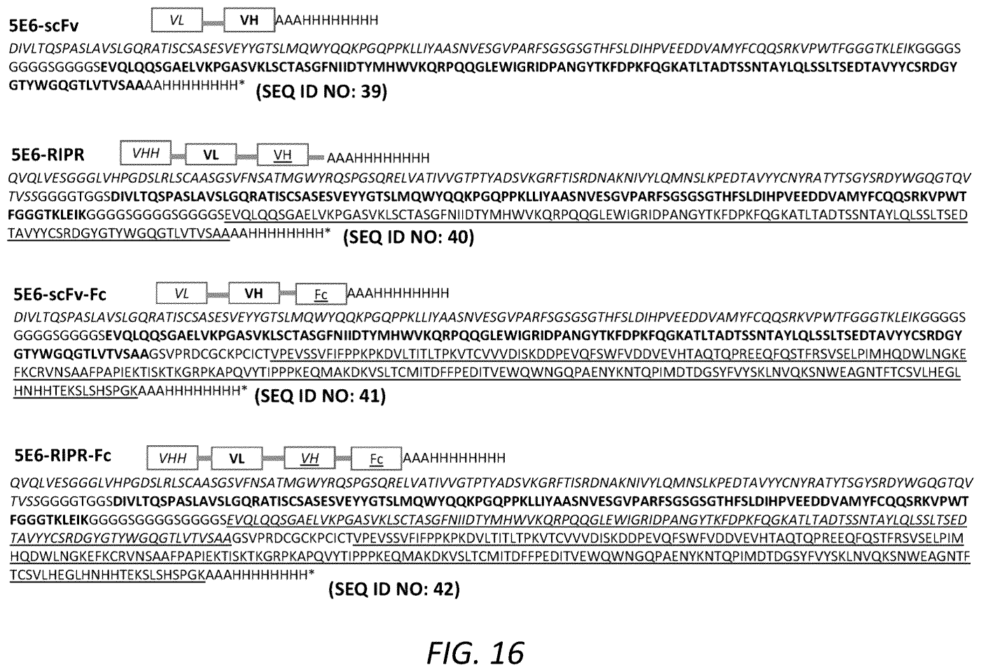

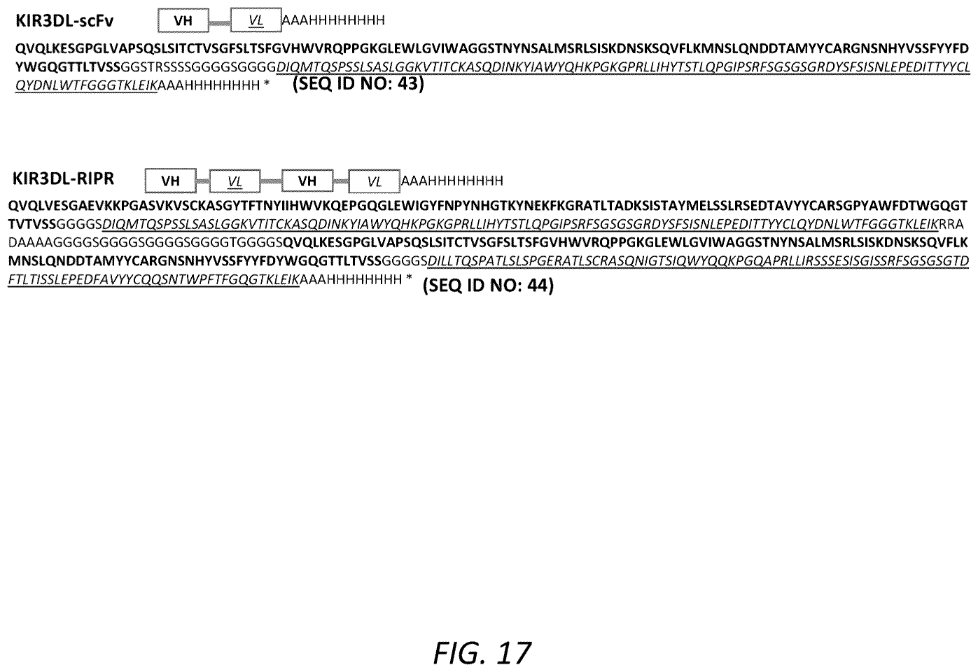

The amino acid sequences of various polypeptides described in these experiments, including anti-NKG2A scFv (clone 16A11), 16A11-RIPR, 16A11-scFv-Fc, 16A11-RIPR-Fc, anti-NKG2A scFv (clone 20D5), 20D5-RIPR, 20D5-scFv-Fc, 20D5-RIPR-Fc, anti-Ly49C/I scFv (clone 5E6), 5E6-RIPR, 5E6-scFv-Fc, 5E6-RIPR-Fc, anti-KIR3DL scFv (clone AZ158), and KIR3D-RIPR are shown in FIGS. 14-17.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

What is claimed is:

1. A multivalent polypeptide comprising:
a first amino acid sequence comprising a first polypeptide module capable of binding to a NK cell receptor (NKR) that signals through a phosphorylation mechanism; and
a second amino acid sequence comprising a second polypeptide module capable of binding to one or more receptor protein-tyrosine phosphatases (RPTPs) expressed on an immune cell that also expresses the NKR.

2. The multivalent polypeptide of claim 1, wherein the immune cell is a natural killer (NK) cell or a T cell.

3. The multivalent polypeptide of any one of claims 1 to 2, wherein the immune cell is a NK cell.

4. The multivalent polypeptide of any one of claims 1 to 2, wherein the immune cell is a T cell.

5. The multivalent polypeptide of claim 4, wherein the T cell is a CD8+ T cell.

6. The multivalent polypeptide of any one of claims 1 to 5, wherein the one or more RPTPs comprises CD45, CD148, or a functional variant of any thereof.

7. The multivalent polypeptide of any one of claims 1-6, wherein the NKR is an inhibiting NKR.

8. The multivalent polypeptide of claim 7, wherein the inhibiting NKR is selected from the group consisting of killer immunoglobulin receptors KIR2DL, KIR3DL, NKG2A, NKG2B, NKG2E, NKG2F, NKp44, NKp30c, CD160, LAIR1, TIM-3, CD96, CEACAM1 (CEACAM5), KLRG-1, and TIGIT.

9. The multivalent polypeptide of any one of claims 1 to 8, wherein the NKR is an activating NKR.

10. The multivalent polypeptide of claim 9, wherein the activating NKR is selected from the group consisting of NKp30a, NKp30b, NKp44, NKp46, NKG2D, NKG2C, KIR2DS, KIR3DS, KIR3DL4, DNAM-1, CD16, and CD161.

11. The multivalent polypeptide of any one of claims 1-10, wherein at least one of the first and second polypeptide modules comprises an amino acid sequence for a protein-binding ligand or an antigen-binding moiety.

12. The multivalent polypeptide of claim 11, wherein the antigen-binding moiety is selected from the group consisting of a single-chain variable fragment (scFv), an antigen-binding fragment (Fab), a nanobody, a V_{H} domain, a V_{L} domain, a single domain antibody (dAb), a V_{NAR} domain, and a V_{H}H domain, a diabody, or a functional fragment of any thereof.

13. The multivalent polypeptide of claim 11, wherein the protein-binding ligand comprises an extracellular domain (ECD) of a NKR's natural ligand, an ECD of a cell surface receptor, or an ECD of a RPTP, or a functional variant of any thereof.

14. The multivalent polypeptide of claim 13, wherein the protein-binding ligand comprises one or more ECD of an MHC-I molecule (HLA) or a functional variant thereof.

15. The multivalent polypeptide of any one of claims 1-14, wherein the first polypeptide module is operably linked to the second polypeptide module via a polypeptide linker sequence.

16. The multivalent polypeptide of any one of claims 1-15, wherein the multivalent polypeptide further comprises an Fc region.

17. The multivalent polypeptide of claim 16, wherein the Fc region is operably linked to the multivalent polypeptide via a polypeptide linker sequence.

18. The multivalent polypeptide of any one of claims 1-1715, further comprising a third amino acid sequence comprising a third polypeptide module capable of binding to an antigen expressed on a CD8+ T cell.

19. The multivalent polypeptide of any one of claims 1-18, wherein the multivalent polypeptide comprises:
(a) (i) an ECD of an MHC-I molecule, (ii) a polypeptide linker, and (iii) a CD45 scFv;
(b) (i) a KIR2DL scFv, (ii) a polypeptide linker; and (iii) a CD45 scFv;
(c) (i) a NKG2A scFv, (ii) a polypeptide linker; and (iii) a CD45 scFv;
(d) (i) a KIR3DL scFv, (ii) a polypeptide linker; and (iii) a CD45 scFv; or
(e) (i) a Ly49C/I scFv, (ii) a polypeptide linker; and (iii) a CD45 scFv.

20. The multivalent polypeptide of claim 19, wherein the multivalent polypeptide further comprises an Fc region.

21. The multivalent polypeptide of any one of claims 1-20, wherein the multivalent polypeptide comprises an amino acid sequence that has at least 80% sequence identity to an amino acid sequence selected from the group consisting of SEQ ID NOS: 1-2, 32, 34, 36, 38, 40, 42, and 44.

22. A recombinant nucleic acid molecule comprising a nucleotide sequence encoding a multivalent polypeptide according to any one of claims 1 to 21.

23. The recombinant nucleic acid molecule of claim 22, wherein the nucleotide sequence has at least 80% sequence identity to a nucleotide sequence selected from the group consisting of SEQ ID NOS: 5-6.

24. A recombinant cell comprising:
(a) a multivalent polypeptide according to any one of claims 1 to 21, and/or
(b) a recombinant nucleic acid molecule of any one of claims 22 to 23.

25. The recombinant cell of claim 24, wherein the recombinant cell is an immune cell.

26. The recombinant cell of claim 25, wherein the immune cell expresses an NKR.

27. The recombinant cell of any one of claims 25 to 26, wherein the immune cell is a NK cell or a T cell.

28. The recombinant cell of claim 27, wherein the T cell is a NK cell

29. The recombinant cell of claim 27, wherein the T cell is a CD8+ T cell.

30. A pharmaceutical composition comprising a pharmaceutical acceptable excipient, and:
a) a multivalent polypeptide according to any one of claims 1 to 21;
b) a recombinant nucleic acid molecule according to any one of claims 22 to 23; and/or
c) a recombinant cell according to any one of claims 24 to 29.

31. A method for modulating cell signaling mediated by a NKR in a subject, the method comprising administering to the subject a composition comprising:
(a) a multivalent polypeptide according to any one of claims 1 to 21;
(b) a recombinant nucleic acid molecule according to any one of claims 22 to 23;
(c) a recombinant cell according to any one of claims 24 to 29; and/or
(d) a pharmaceutical composition of claim 30.

32. A method for treating a health condition in a subject in need thereof, the method comprising administering to the subject a composition comprising:
(a) a multivalent polypeptide according to any one of claims 1 to 21;
(b) a recombinant nucleic acid molecule according to any one of claims 22 to 23; and/or
(c) a recombinant cell according to any one of claims 24 to 29; and/or
(d) a pharmaceutical composition of claim 30.

33. The method of any one of claims 31-32, wherein the administered composition recruits the RPTP activity to a spatial proximity of a NKR, potentiates dephosphorylation of a NKR, and/or reduces NKR-mediated signaling.

34. The method of any one of claims 31-33, wherein the administered composition confers an enhancement in NK cell killing of a target cell.

35. The method of any one of claims 31-34, wherein the subject has or is suspected of having a health condition associated with a natural killer cell receptor.

36. The method of claim 35, wherein the health condition is a cancer, an autoimmune disease, or a viral infection.

37. The method of any one of claim 32-36, the composition is administered to the subject individually (monotherapy) or as a first therapy in combination with a second therapy.

38. The method of claim 37, wherein the second therapy is selected from the group consisting of chemotherapy, radiotherapy, immunotherapy, hormonal therapy, toxin therapy, or surgery.

39. The method of any one of claims 37 to 38, wherein the second therapy comprises an anti-NKR antagonistic antibody.

40. A kit for modulating cell signaling mediated by a NKR in a subject, or for treating a health condition in a subject in need thereof, the kit comprising instructions for use thereof and one or more of the following:
(a) a multivalent polypeptide according to any one of claims 1 to 21;
(b) a recombinant nucleic acid molecule according to any one of claims 22 to 23; and/or
(c) a recombinant cell according to any one of claims 24 to 29; and
(d) a pharmaceutical composition of claim 30.


SEQUENCE LISTING (47 sequences)

SEQ ID NO: 1 - protein, 511 aa, Artificial sequence
  Synthetic construct
  MISC_FEATURE: NKG2A-RIPR Polypeptide
  MISC_FEATURE (1)..(118): VH
  MISC_FEATURE (119)..(122): linker
  MISC_FEATURE (123)..(229): VL
  MISC_FEATURE (230)..(262): linker
  MISC_FEATURE (263)..(387): VH
  MISC_FEATURE (388)..(392): linker
  MISC_FEATURE (393)..(399): VL
QVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSSGGGGSDIQMTQSPASLSASVGETVTITCRASENIYSYLAWYQQKQGKSPQFLVYNAKTLAEGVPSRFSGSGSGTQFSLKINSLQPEDFGSYYCQHHYGTPRTFGGGTKLEIKRRADAAAAGGGGSGGGGSGGGGSGGGGTGGGGSQVQLQQPGAELVRPGASVKLSCKASGYTFTSYWMNWVKQRPEQGLQWIGRIDPYDSETHYSQKFKDKAILTVDKSSSTAYMRLSSLTSEDSAVYYCARGGYDFDVGTLYWFFDVWGAGTTVTVSSGGGGSDILLTQSPATLSLSPGERATLSCRASQNIGTSIQWYQQKPGQAPRLLIRSSSESISGISSRFSGSGSGTDFTLTISSLEPEDFAVYYCQQSNTWPFTFGQGTKLEIKAAAHHHHHHHH

SEQ ID NO: 2 - protein, 511 aa, Artificial sequence
  Synthetic construct
  MISC_FEATURE: KIR2DL-RIPR Polypeptide
  MISC_FEATURE (1)..(118): VH
  MISC_FEATURE (119)..(123): linker
  MISC_FEATURE (124)..(232): VL
  MISC_FEATURE (233)..(265): linker
  MISC_FEATURE (266)..(388): VH
  MISC_FEATURE (389)..(393): linker
  MISC_FEATURE (394)..(500): VL
QVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSSGGGGSEIVLTQSPVTLSLSPGERATLSCRASQSVSSYLAWYQQKPGQAPRLLIYDASNRATGIPARFSGSGSGTDFTLTISSLEPEDFAVYYCQQRSNWMYTFGQGTKLEIKRTRRADAAAAGGGGSGGGGSGGGGSGGGGTGGGGSQVQLVQSGAEVKKPGSSVKVSCKASGGTFSFYAISWVRQAPGQGLEWMGGFIPIFGAANYAQKFQGRVTITADESTSTAYMELSSLRSDDTAVYYCARIPSGSYYYDYDMDVWGQGTTVTVSSGGGGSDILLTQSPATLSLSPGERATLSCRASQNIGTSIQWYQQKPGQAPRLLIRSSSESISGISSRFSGSGSGTDFTLTISSLEPEDFAVYYCQQSNTWPFTFGQGTKLEIKAAAHHHHHHHH

SEQ ID NO: 3 - protein, 261 aa, Artificial sequence
  Synthetic construct
  MISC_FEATURE: Anti-NKG2A scFv Polypeptide
  MISC_FEATURE (1)..(125): VH
  MISC_FEATURE (126)..(143): linker
  MISC_FEATURE (144)..(250): VL
QVQLQQPGAELVRPGASVKLSCKASGYTFTSYWMNWVKQRPEQGLQWIGRIDPYDSETHYSQKFKDKAILTVDKSSSTAYMRLSSLTSEDSAVYYCARGGYDFDVGTLYWFFDVWGAGTTVTVSSGGSTRSSSSGGGGSGGGGDIQMTQSPASLSASVGETVTITCRASENIYSYLAWYQQKQGKSPQFLVYNAKTLAEGVPSRFSGSGSGTQFSLKINSLQPEDFGSYYCQHHYGTPRTFGGGTKLEIKAAAHHHHHHHH

SEQ ID NO: 4 - protein, 261 aa, Artificial sequence
  Synthetic construct
  MISC_FEATURE: Anti-KIR2DL scFv Polypeptide
  MISC_FEATURE (1)..(122): VH
  MISC_FEATURE (123)..(141): linker
  MISC_FEATURE (142)..(250): VL
QVQLVQSGAEVKKPGSSVKVSCKASGGTFSFYAISWVRQAPGQGLEWMGGFIPIFGAANYAQKFQGRVTITADESTSTAYMELSSLRSDDTAVYYCARIPSGSYYYDYDMDVWGQGTTVTVSSGGSTRSSSSGGGGSGGGGEIVLTQSPVTLSLSPGERATLSCRASQSVSSYLAWYQQKPGQAPRLLIYDASNRATGIPARFSGSGSGTDFTLTISSLEPEDFAVYYCQQRSNWMYTFGQGTKLEIKRTAAAHHHHHHHH

SEQ ID NO: 5 - DNA, 1536 bp, Artificial sequence
  Synthetic construct
  misc_feature: Nucleotide sequence encoding the NKG2A-RIPR construct of SEQ ID NO 1
caagtgcagctggtagaaagtggcgcggaagtaaaaaagcccggtgcctctgtcaaagtttcttgcaaagccagtggttatacgtttacgaactacatcattcactgggttaagcaggaacccggacaaggcctcgaatggattggatatttcaacccctacaaccacggcaccaagtataacgaaaaattcaagggaagggcgacgctgacagcggataaatccatctctaccgcgtatatggaactgagctcattgaggagtgaagacactgcagtctactattgcgctcgcagcggcccatacgcatggtttgacacttggggtcaaggaactaccgttaccgtcagttccggtggcggaggaagcgatattcaaatgacacagtctcctgcctcactcagtgcctcagtgggcgagaccgtaacgattacttgcagggcctccgaaaacatatattcttatcttgcgtggtatcaacagaaacaaggtaaaagcccgcaatttttggtttataatgcaaagacccttgctgaaggcgtgccatccaggttttctggaagcggatctggcacacagtttagcctcaagataaatagtctccaaccggaggattttggtagttattattgccaacatcactatggcacacctcgtacatttggtggtggcactaaactggaaatcaaacgccgcgcagacgctgccgcggcaggaggaggaggatcaggtggtggaggatctggaggaggaggtagcggaggtggtggaaccggcggaggaggttcacaagtacagcttcaacagcctggagcggaattggttcgcccaggagcaagcgtcaagctgtcctgcaaagcaagtggttacacatttactagttattggatgaattgggtaaaacagcgcccagagcaaggacttcagtggattggaaggatagatccgtatgacagtgaaacacactacagtcaaaagtttaaggataaagcgatcttgacagttgataagagcagcagcacagcatatatgcgcttgagttccctgacctctgaggatagtgccgtatattattgcgcaagaggcggttacgacttcgacgtcggcaccttgtactggttctttgatgtttggggtgctggcactactgtgacagtatcctcaggtggaggaggcagcgatatcctgttgacccaaagccctgcaacgcttagcttgtctcccggtgagcgtgccacactttcttgtagagcatcacagaacattggaacatctatccaatggtaccaacagaagccgggacaagcgccccgtcttctgatacgctcctcaagcgagtcaattagcggcataagtagcagattcagcggatccggatccggcacggactttacccttactataagttccctcgagccggaggatttcgccgtctactattgccagcaaagcaatacttggccgttcactttcggtcaaggcactaaattggagattaaggcggccgcgcatcatcaccaccatcaccaccattaa

SEQ ID NO: 6 - DNA, 1536 bp, Artificial sequence
  Synthetic construct
  misc_feature: Nucleotide sequence encoding the KIR2DL-RIPR construct of SEQ ID NO 2
caagtacagctcgtagagagtggcgcagaagttaaaaagcccggcgcatctgttaaggtgagctgtaaagccagcggctatacctttactaactatatcatccattgggtaaaacaagaaccgggccagggattggagtggattggatattttaacccgtacaatcatggtacgaagtacaatgagaaattcaagggacgtgcgactctcacagccgataagagcatatcaacggcatatatggaactctccagcttgaccagtgaagacaccgcggtttactactgcgctaggtcaggaccctacgcctggtttgacacatggggtcaaggtaccaccgtcaccgttagctccggaggcggtggcagcgagatcgttttgactcaatccccggtaactctttcactgtcaccaggtgagagagctacattgtcctgccgcgcatcccaatcagttagttcttatcttgcgtggtaccagcaaaagccgggacaagctccaagactccttatatatgacgcgtccaacagggcgacaggaataccggctagattttccggttctggtagtggcactgacttcaccctgaccatatcatccctcgagcctgaggattttgcagtatattattgccaacaaaggtccaattggatgtacacattcggccaaggcactaaattggaaataaagcgcacgagacgtgccgacgcagcagccgcgggaggtggtggctcaggcggcggaggatctggtggcggaggttctggaggcggcggtaccggtggcggtggcagtcaggttcaactggtacagagcggcgccgaggtcaagaagccgggatcttcagtcaaagtcagctgtaaagctagcggaggtactttcagcttctatgcaatatcctgggtaaggcaggcgcccggacaaggcctggagtggatgggcggttttatccccattttcggcgcggccaattatgctcagaagtttcaaggtagggttactataaccgctgacgaatcaacatccacagcttatatggaactttcttctcttacctctgatgataccgcagtctactattgcgcacgcataccgagtggcagctactattacgactacgatatggacgtctggggccagggtaccaccgttacagttagctctggcggaggaggttctgacattctgctcacacagtctcctgcgacactctcactttcccccggagagcgcgctacactgagctgccgtgcctctcagaatattggtacttctattcagtggtaccaacaaaaacctggccaagccccacgccttcttattcgctccagctctgagagcataagtggcatttcctcacgcttttcaggatccggttccggaactgacttcactctcaccatctcctccttggaacccgaagattttgctgtttactattgtcagcaaagtaatacttggccctttacgtttggtcaaggaaccaaacttgaaatcaaggcggccgcgcatcatcaccaccatcaccaccattaa

SEQ ID NO: 7 - protein, 18 aa, Artificial sequence
  Synthetic construct
  MISC_FEATURE: Polypeptide linker
GGSTRSSSSGGGGSGGGG

SEQ ID NO: 8 - protein, 5 aa, Artificial sequence
  Synthetic construct
  MISC_FEATURE: Polypeptide linker
GGGGS

SEQ ID NO: 9 - protein, 33 aa, Artificial sequence
  Synthetic construct
  MISC_FEATURE: Polypeptide linker
RRADAAAAGGGGSGGGGSGGGGSGGGGTGGGGS

SEQ ID NO: 10 - protein, 11 aa, Artificial sequence
  Synthetic construct
  MISC_FEATURE (1)..(1): Xaa is either Isoleucine or Valine
  misc_feature (4)..(4): Xaa can be any naturally occurring amino acid
  misc_feature (7)..(8): Xaa can be any naturally occurring amino acid
  MISC_FEATURE (10)..(10): Xaa is either Serine or Threonine
XHCXAGXXRXG

SEQ ID NO: 11 - protein, 25 aa, Artificial sequence
  Synthetic construct
  MISC_FEATURE (1)..(25): Xaa can be any naturally occurring amino acid
  MISC_FEATURE (1)..(20): Bases 1-20 may be present or absent
GGGXXGGGXXGGGXXGGGXXGGGGS

SEQ ID NO: 12 - protein, 25 aa, Artificial sequence
  Synthetic construct
  MISC_FEATURE (1)..(25): Xaa can be any naturally occurring amino acid
  MISC_FEATURE (6)..(6): Bases 6-25 may be present or absent
GGGGSXGGGSXGGGSXGGGSXGGGS

SEQ ID NO: 13 - protein, 25 aa, Artificial sequence
  Synthetic construct
  MISC_FEATURE (1)..(20): Bases 1-20 may be present or absent
  MISC_FEATURE (4)..(4): Xaa is Proline
  MISC_FEATURE (5)..(5): Xaa is Serine
  MISC_FEATURE (9)..(9): Xaa is Proline
  MISC_FEATURE (10)..(10): Xaa is Serine
  MISC_FEATURE (14)..(14): Xaa is Proline
  MISC_FEATURE (15)..(15): Xaa is Serine
  MISC_FEATURE (19)..(19): Xaa is Proline
  MISC_FEATURE (20)..(20): Xaa is Serine
GGGXXGGGXXGGGXXGGGXXGGGGS

SEQ ID NO: 14 - protein, 25 aa, Artificial sequence
  Synthetic construct
  MISC_FEATURE (1)..(20): Bases 1-20 may be present or absent
  MISC_FEATURE (4)..(4): Xaa is Glycine
  MISC_FEATURE (5)..(5): Xaa is Glutamine
  MISC_FEATURE (9)..(9): Xaa is Glycine
  MISC_FEATURE (10)..(10): Xaa is Glutamine
  MISC_FEATURE (14)..(14): Xaa is Glycine
  MISC_FEATURE (15)..(15): Xaa is Glutamine
  MISC_FEATURE (19)..(19): Xaa is Glycine
  MISC_FEATURE (20)..(20): Xaa is Glutamine
GGGXXGGGXXGGGXXGGGXXGGGGS

SEQ ID NO: 15 - protein, 25 aa, Artificial sequence
  Synthetic construct
  MISC_FEATURE (1)..(20): Bases 1-20 may be present or absent
  MISC_FEATURE (4)..(4): Xaa is Glycine
  MISC_FEATURE (5)..(5): Xaa is Alanine
  MISC_FEATURE (9)..(9): Xaa is Glycine
  MISC_FEATURE (10)..(10): Xaa is Alanine
  MISC_FEATURE (14)..(14): Xaa is Glycine
  MISC_FEATURE (15)..(15): Xaa is Alanine
  MISC_FEATURE (19)..(19): Xaa is Glycine
  MISC_FEATURE (20)..(20): Xaa is Alanine
GGGXXGGGXXGGGXXGGGXXGGGGS

SEQ ID NO: 16 - protein, 25 aa, Artificial sequence
  Synthetic construct
  MISC_FEATURE (1)..(25): Xaa is Proline
  MISC_FEATURE (6)..(25): Bases 6-25 may be present or absent
GGGGSXGGGSXGGGSXGGGSXGGGS

SEQ ID NO: 17 - protein, 15 aa, Artificial sequence
  Synthetic construct
GGGGAGGGGAGGGGS

SEQ ID NO: 18 - protein, 15 aa, Artificial sequence
  Synthetic construct
GGGGQGGGGQGGGGS

SEQ ID NO: 19 - protein, 15 aa, Artificial sequence
  Synthetic construct
GGGPSGGGPSGGGGS

SEQ ID NO: 20 - protein, 15 aa, Artificial sequence
  Synthetic construct
GGGGSPGGGSPGGGS

SEQ ID NO: 21 - protein, 5 aa, Artificial sequence
  Synthetic construct
GSGGS

SEQ ID NO: 22 - protein, 6 aa, Artificial sequence
  Synthetic construct
SGGSGS

SEQ ID NO: 23 - protein, 5 aa, Artificial sequence
  Synthetic construct
GGGGS

SEQ ID NO: 24 - protein, 5 aa, Artificial sequence
  Synthetic construct
SGGGG

SEQ ID NO: 25 - protein, 6 aa, Artificial sequence
  Synthetic construct
GGGGGS

SEQ ID NO: 26 - protein, 6 aa, Artificial sequence
  Synthetic construct
SGGGGG

SEQ ID NO: 27 - protein, 7 aa, Artificial sequence
  Synthetic construct
GGGGGGS

SEQ ID NO: 28 - protein, 7 aa, Artificial sequence
  Synthetic construct
SGGGGGG

SEQ ID NO: 29 - protein, 250 aa, Artificial sequence
  Synthetic construct
  MISC_FEATURE: Bases 1-250 may be present or absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 30 - protein, 250 aa, Artificial sequence
  Synthetic construct
  MISC_FEATURE (1)..(250): Bases 1-250 may be present or absent
SGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGG

SEQ ID NO: 31 - protein, 255 aa, Artificial sequence
  Synthetic construct
  MISC_FEATURE: 16A11-scFv Polypeptide
  misc_feature (1)..(112): VL
  misc_feature (113)..(127): linker
  misc_feature (128)..(244): VH
DVVMTQTPLSLPVSLGDQASISCRSSQSLVHSNGNTYLHWYLQKPGQSPKLLIYKVYNRFSGVPDRFSGSGSGTDFTLKISRVEAEDLGVYFCSQSTHVPWTFGGGTKLEIKGGGGSGGGGSGGGGSEVQLQQSGTVLARPGASVKMSCKASGYRFTSYWMHWVKQRPGQGLEWIGGIYLGNSDTNYNQKFKGKAKLTAVTSASTAYVEVSSLTIEDSAVYYCSRLDLGAFDYWGQGTTLTVSSAAAHHHHHHHH

SEQ ID NO: 32 - protein, 382 aa, Artificial sequence
  Synthetic polypeptide
  MISC_FEATURE: 16A11-RIPR Polypeptide
  misc_feature (1)..(119): VHH
  misc_feature (120)..(127): linker
  misc_feature (128)..(239): VL
  misc_feature (240)..(254): linker
  misc_feature (255)..(371): VH
QVQLVESGGGLVHPGDSLRLSCAASGSVFNSATMGWYRQSPGSQRELVATIVVGTPTYADSVKGRFTISRDNAKNIVYLQMNSLKPEDTAVYYCNYRATYTSGYSRDYWGQGTQVTVSSGGGGTGGSDVVMTQTPLSLPVSLGDQASISCRSSQSLVHSNGNTYLHWYLQKPGQSPKLLIYKVYNRFSGVPDRFSGSGSGTDFTLKISRVEAEDLGVYFCSQSTHVPWTFGGGTKLEIKGGGGSGGGGSGGGGSEVQLQQSGTVLARPGASVKMSCKASGYRFTSYWMHWVKQRPGQGLEWIGGIYLGNSDTNYNQKFKGKAKLTAVTSASTAYVEVSSLTIEDSAVYYCSRLDLGAFDYWGQGTTLTVSSAAAHHHHHHHH

SEQ ID NO: 33 - protein, 484 aa, Artificial sequence
  Synthetic polypeptide
  MISC_FEATURE: 16A11-scFv-Fc Polypeptide
  misc_feature (1)..(112): VL
  misc_feature (113)..(127): linker
  misc_feature (128)..(244): VH
  misc_feature (245)..(259): linker
  misc_feature (260)..(473): Fc
DVVMTQTPLSLPVSLGDQASISCRSSQSLVHSNGNTYLHWYLQKPGQSPKLLIYKVYNRFSGVPDRFSGSGSGTDFTLKISRVEAEDLGVYFCSQSTHVPWTFGGGTKLEIKGGGGSGGGGSGGGGSEVQLQQSGTVLARPGASVKMSCKASGYRFTSYWMHWVKQRPGQGLEWIGGIYLGNSDTNYNQKFKGKAKLTAVTSASTAYVEVSSLTIEDSAVYYCSRLDLGAFDYWGQGTTLTVSSGSVPRDCGCKPCICTVPEVSSVFIFPPKPKDVLTITLTPKVTCVVVDISKDDPEVQFSWFVDDVEVHTAQTQPREEQFQSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKAAAHHHHHHHH

SEQ ID NO: 34 - protein, 611 aa, Artificial sequence
  Synthetic polypeptide
  MISC_FEATURE: 16A11-RIPR-Fc Polypeptide
  misc_feature (1)..(119): VHH
  misc_feature (120)..(127): linker
  misc_feature (128)..(239): VL
  misc_feature (240)..(254): linker
  misc_feature (255)..(371): VH
  misc_feature (372)..(386): linker
  misc_feature (387)..(600): Fc
QVQLVESGGGLVHPGDSLRLSCAASGSVFNSATMGWYRQSPGSQRELVATIVVGTPTYADSVKGRFTISRDNAKNIVYLQMNSLKPEDTAVYYCNYRATYTSGYSRDYWGQGTQVTVSSGGGGTGGSDVVMTQTPLSLPVSLGDQASISCRSSQSLVHSNGNTYLHWYLQKPGQSPKLLIYKVYNRFSGVPDRFSGSGSGTDFTLKISRVEAEDLGVYFCSQSTHVPWTFGGGTKLEIKGGGGSGGGGSGGGGSEVQLQQSGTVLARPGASVKMSCKASGYRFTSYWMHWVKQRPGQGLEWIGGIYLGNSDTNYNQKFKGKAKLTAVTSASTAYVEVSSLTIEDSAVYYCSRLDLGAFDYWGQGTTLTVSSGSVPRDCGCKPCICTVPEVSSVFIFPPKPKDVLTITLTPKVTCVVVDISKDDPEVQFSWFVDDVEVHTAQTQPREEQFQSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKAAAHHHHHHHH

SEQ ID NO: 35 - protein, 252 aa, Artificial sequence
  Synthetic polypeptide
  MISC_FEATURE: 20D5-scFv Polypeptide
  misc_feature (1)..(107): VL
  misc_feature (108)..(122): linker
  misc_feature (123)..(241): VH
DIVMSQSPTSMSISEGDRVTMNCKASQIVGANVDWYQQKIGQSPKLLIFKTFNRYTGVPDRFTGSGSGTDFTFSISNMQAEDLAVYYCMQSHSYPYTFGSGTKLEIKGGGGSGGGGSGGGGSEEQLVESGGGLVQPGGSLKLSCVASGFTFNSFWMTWIRQAPGKGLEWVASITNTGGRTYYPDSLRGRFTISRDNGKSTLYLQMSSLRSEDTATYYCTRDLPGYNVMDAWGQGASVTVSSAAAHHHHHHHH

SEQ ID NO: 36 - protein, 379 aa, Artificial sequence
  Synthetic polypeptide
  MISC_FEATURE: 20D5-RIPR Polypeptide
  misc_feature (1)..(120): VH
  misc_feature (121)..(127): linker
  misc_feature (128)..(234): VL
  misc_feature (235)..(249): linker
  misc_feature (250)..(380): VH
QVQLVESGGGLVHPGDSLRLSCAASGSVFNSATMGWYRQSPGSQRELVATIVVGTPTYADSVKGRFTISRDNAKNIVYLQMNSLKPEDTAVYYCNYRATYTSGYSRDYWGQGTQVTVSSGGGGTGGSDIVMSQSPTSMSISEGDRVTMNCKASQIVGANVDWYQQKIGQSPKLLIFKTFNRYTGVPDRFTGSGSGTDFTFSISNMQAEDLAVYYCMQSHSYPYTFGSGTKLEIKGGGGSGGGGSGGGGSEEQLVESGGGLVQPGGSLKLSCVASGFTFNSFWMTWIRQAPGKGLEWVASITNTGGRTYYPDSLRGRFTISRDNGKSTLYLQMSSLRSEDTATYYCTRDLPGYNVMDAWGQGASVTVSSAAAHHHHHHHH

SEQ ID NO: 37 - protein, 481 aa, Artificial sequence
  Synthetic polypeptide
  MISC_FEATURE: 20D5-scFv-Fc Polypeptide
  misc_feature (1)..(107): VL
  misc_feature (108)..(122): linker
  misc_feature (123)..(241): VH
  misc_feature (242)..(256): linker
  misc_feature (257)..(470): Fc
DIVMSQSPTSMSISEGDRVTMNCKASQIVGANVDWYQQKIGQSPKLLIFKTFNRYTGVPDRFTGSGSGTDFTFSISNMQAEDLAVYYCMQSHSYPYTFGSGTKLEIKGGGGSGGGGSGGGGSEEQLVESGGGLVQPGGSLKLSCVASGFTFNSFWMTWIRQAPGKGLEWVASITNTGGRTYYPDSLRGRFTISRDNGKSTLYLQMSSLRSEDTATYYCTRDLPGYNVMDAWGQGASVTVSSGSVPRDCGCKPCICTVPEVSSVFIFPPKPKDVLTITLTPKVTCVVVDISKDDPEVQFSWFVDDVEVHTAQTQPREEQFQSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKAAAHHHHHHHH

SEQ ID NO: 38 - protein, 608 aa, Artificial sequence
  Synthetic polypeptide
  MISC_FEATURE: 20D5-RIPR-Fc Polypeptide
  misc_feature (1)..(119): VH
  misc_feature (120)..(127): linker
  misc_feature (128)..(234): VL
  misc_feature (235)..(249): linker
  misc_feature (250)..(368): VH
  misc_feature (369)..(383): linker
  misc_feature (384)..(597): Fc
QVQLVESGGGLVHPGDSLRLSCAASGSVFNSATMGWYRQSPGSQRELVATIVVGTPTYADSVKGRFTISRDNAKNIVYLQMNSLKPEDTAVYYCNYRATYTSGYSRDYWGQGTQVTVSSGGGGTGGSDIVMSQSPTSMSISEGDRVTMNCKASQIVGANVDWYQQKIGQSPKLLIFKTFNRYTGVPDRFTGSGSGTDFTFSISNMQAEDLAVYYCMQSHSYPYTFGSGTKLEIKGGGGSGGGGSGGGGSEEQLVESGGGLVQPGGSLKLSCVASGFTFNSFWMTWIRQAPGKGLEWVASITNTGGRTYYPDSLRGRFTISRDNGKSTLYLQMSSLRSEDTATYYCTRDLPGYNVMDAWGQGASVTVSSGSVPRDCGCKPCICTVPEVSSVFIFPPKPKDVLTITLTPKVTCVVVDISKDDPEVQFSWFVDDVEVHTAQTQPREEQFQSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKAAAHHHHHHHH

SEQ ID NO: 39 - protein, 252 aa, Artificial sequence
  Synthetic polypeptide
  MISC_FEATURE: 5E6-scFv Polypeptide
  misc_feature (1)..(111): VL
  misc_feature (112)..(126): linker
  misc_feature (127)..(242): VH
DIVLTQSPASLAVSLGQRATISCSASESVEYYGTSLMQWYQQKPGQPPKLLIYAASNVESGVPARFSGSGSGTHFSLDIHPVEEDDVAMYFCQQSRKVPWTFGGGTKLEIKGGGGSGGGGSGGGGSEVQLQQSGAELVKPGASVKLSCTASGFNIIDTYMHWVKQRPQQGLEWIGRIDPANGYTKFDPKFQGKATLTADTSSNTAYLQLSSLTSEDTAVYYCSRDGYGTYWGQGTLVTVSAAAAHHHHHHHH

SEQ ID NO: 40 - protein, 379 aa, Artificial sequence
  Synthetic polypeptide
  MISC_FEATURE: 5E6-RIPR Polypeptide
  misc_feature (1)..(117): VHH
  misc_feature (118)..(125): linker
  misc_feature (126)..(238): VL
  misc_feature (239)..(253): linker
  misc_feature (254)..(369): VH
QVQLVESGGGLVHPGDSLRLSCAASGSVFNSATMGWYRQSPGSQRELVATIVVGTPTYADSVKGRFTISRDNAKNIVYLQMNSLKPEDTAVYYCNYRATYTSGYSRDYWGQGTQVTVSSGGGGTGGSDIVLTQSPASLAVSLGQRATISCSASESVEYYGTSLMQWYQQKPGQPPKLLIYAASNVESGVPARFSGSGSGTHFSLDIHPVEEDDVAMYFCQQSRKVPWTFGGGTKLEIKGGGGSGGGGSGGGGSEVQLQQSGAELVKPGASVKLSCTASGFNIIDTYMHWVKQRPQQGLEWIGRIDPANGYTKFDPKFQGKATLTADTSSNTAYLQLSSLTSEDTAVYYCSRDGYGTYWGQGTLVTVSAAAAHHHHHHHH

SEQ ID NO: 41 - protein, 482 aa, Artificial sequence
  Synthetic polypeptide
  MISC_FEATURE: 5E6-scFv-Fc Polypeptide
  misc_feature (1)..(112): VL
  misc_feature (113)..(126): linker
  misc_feature (127)..(222): VH
  misc_feature (223)..(237): linker
  misc_feature (238)..(451): Fc
DIVLTQSPASLAVSLGQRATISCSASESVEYYGTSLMQWYQQKPGQPPKLLIYAASNVESGVPARFSGSGSGTHFSLDIHPVEEDDVAMYFCQQSRKVPWTFGGGTKLEIKGGGGSGGGGSGGGGSEVQLQQSGAELVKPGASVKLSCTASGFNIIDTYMHWVKQRPQQGLEWIGRIDPANGYTKFDPKFQGKATLTADTSSNTAYLQLSSLTSEDTAVYYCSRDGYGTYWGQGTLVTVSAAGSVPRDCGCKPCICTVPEVSSVFIFPPKPKDVLTITLTPKVTCVVVDISKDDPEVQFSWFVDDVEVHTAQTQPREEQFQSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKAAAHHHHHHHH

SEQ ID NO: 42 - protein, 609 aa, Artificial sequence
  Synthetic polypeptide
  MISC_FEATURE: 5E6-RIPR-Fc Polypeptide
  misc_feature (1)..(119): VHH
  misc_feature (120)..(127): linker
  misc_feature (128)..(238): VL
  misc_feature (239)..(253): linker
  misc_feature (254)..(369): VH
  misc_feature (370)..(383): linker
  misc_feature (384)..(598): Fc
QVQLVESGGGLVHPGDSLRLSCAASGSVFNSATMGWYRQSPGSQRELVATIVVGTPTYADSVKGRFTISRDNAKNIVYLQMNSLKPEDTAVYYCNYRATYTSGYSRDYWGQGTQVTVSSGGGGTGGSDIVLTQSPASLAVSLGQRATISCSASESVEYYGTSLMQWYQQKPGQPPKLLIYAASNVESGVPARFSGSGSGTHFSLDIHPVEEDDVAMYFCQQSRKVPWTFGGGTKLEIKGGGGSGGGGSGGGGSEVQLQQSGAELVKPGASVKLSCTASGFNIIDTYMHWVKQRPQQGLEWIGRIDPANGYTKFDPKFQGKATLTADTSSNTAYLQLSSLTSEDTAVYYCSRDGYGTYWGQGTLVTVSAAGSVPRDCGCKPCICTVPEVSSVFIFPPKPKDVLTITLTPKVTCVVVDISKDDPEVQFSWFVDDVEVHTAQTQPREEQFQSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKAAAHHHHHHHH

SEQ ID NO: 43 - protein, 258 aa, Artificial sequence
  Synthetic construct
  MISC_FEATURE: KIR3DL-scFv Polypeptide
  misc_feature (1)..(123): VH
  misc_feature (124)..(141): linker
  misc_feature (142)..(247): VL
QVQLKESGPGLVAPSQSLSITCTVSGFSLTSFGVHWVRQPPGKGLEWLGVIWAGGSTNYNSALMSRLSISKDNSKSQVFLKMNSLQNDDTAMYYCARGNSNHYVSSFYYFDYWGQGTTLTVSSGGSTRSSSSGGGGSGGGGDIQMTQSPSSLSASLGGKVTITCKASQDINKYIAWYQHKPGKGPRLLIHYTSTLQPGIPSRFSGSGSGRDYSFSISNLEPEDITTYYCLQYDNLWTFGGGTKLEIKAAAHHHHHHHH

SEQ ID NO: 44 - protein, 508 aa, Artificial sequence
  Synthetic construct
  MISC_FEATURE: KIR3DL-RIPR-Fc Polypeptide
  misc_feature (1)..(118): VH
  misc_feature (119)..(123): linker
  misc_feature (124)..(229): VL
  misc_feature (230)..(262): linker
  misc_feature (263)..(385): VH
  misc_feature (386)..(390): linker
  misc_feature (391)..(497): VL
QVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSSGGGGSDIQMTQSPSSLSASLGGKVTITCKASQDINKYIAWYQHKPGKGPRLLIHYTSTLQPGIPSRFSGSGSGRDYSFSISNLEPEDITTYYCLQYDNLWTFGGGTKLEIKRRADAAAAGGGGSGGGGSGGGGSGGGGTGGGGSQVQLKESGPGLVAPSQSLSITCTVSGFSLTSFGVHWVRQPPGKGLEWLGVIWAGGSTNYNSALMSRLSISKDNSKSQVFLKMNSLQNDDTAMYYCARGNSNHYVSSFYYFDYWGQGTTLTVSSGGGGSDILLTQSPATLSLSPGERATLSCRASQNIGTSIQWYQQKPGQAPRLLIRSSSESISGISSRFSGSGSGTDFTLTISSLEPEDFAVYYCQQSNTWPFTFGQGTKLEIKAAAHHHHHHHH

SEQ ID NO: 45 - protein, 15 aa, Artificial sequence
  Synthetic construct
  MISC_FEATURE: Polypeptide linker
GSVPRDCGCKPCICT

SEQ ID NO: 46 - protein, 4 aa, Artificial sequence
  Synthetic construct
  MISC_FEATURE: Polypeptide linker
GGGS

SEQ ID NO: 47 - protein, 4 aa, Artificial sequence
  Synthetic construct
  MISC_FEATURE: Polypeptide linker
SGGG